SEQUENCE LISTING (61 sequences)

SEQ ID NO: 1 - protein, 99 aa, synthetic construct
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNL

SEQ ID NO: 2 - protein, 269 aa, synthetic construct
GCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSRLRAPSSSLGAFFAGDLPPPLQAGAA

SEQ ID NO: 3 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 4 - protein, 458 aa, synthetic construct
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 5 - protein, 255 aa, synthetic construct
AGSHMGVSQSPRYKVAKRGQDVALRCDPISGHVSLFWYQQALGQGPEFLTYFQNEAQLDKSGLPSDRFFAERPEGSVSTLKIQRTQQEDSAVYLCASSLGQAYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCTSGDDDDK

SEQ ID NO: 6 - protein, 241 aa, synthetic construct
MNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSFATEAFFGQGTRLTVVEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 7 - protein, 310 aa, synthetic construct [sequence omitted]

SEQ ID NO: 8 - skipped

SEQ ID NO: 9 - protein, 539 aa, Homo sapiens
MGSPEDDLIGIPFPDHSSELLSCLNEQRQLGHLCDLTIRTQGLEYRTHRAVLAACSHYFKKLFTEGGGGAVMGAGGSGTATGGAGAGVCELDFVGPEALGALLEFAYTATLTTSSANMPAVLQAARLLEIPCVIAACMEILQGSGLEAPSPDEDDCERARQYLEAFATATASGVPNGEDSPPQVPLPPPPPPPPRPVARRSRKPRKAFLQTKGARANHLVPEVPTVPAHPLTYEEEEVAGRVGSSGGSGPGDSYSPPTGTASPPEGPQSYEPYEGEEEEEELVYPPAYGLAQGGGPPLSPEELGSDEDAIDPDLMAYLSSLHQDNLAPGLDSQDKLVRKRRSQMPQECPVCHKIIHGAGKLPRHMRTHTGEKPFACEVCGVRFTRNDKLKIHMRKHTGERPYSCPHCPARFLHSYDLKNHMHLHTGDRPYECHLCHKAFAKEDHLQRHLKGQNCLEVRTRRRRKDDAPPHYPPPSTAAASPAGLDLSNGHLDTFRLSLARFWEQSAPTGPPVSTPGPPDDDEEEGAPTTPQAEGAMESS

SEQ ID NO: 10 - skipped

SEQ ID NO: 11 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 12 - skipped

SEQ ID NO: 13 - protein, 518 aa, Homo sapiens
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 14 - skipped

SEQ ID NO: 15 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 16 - skipped

SEQ ID NO: 17 - protein, 458 aa, Homo sapiens; identical to SEQ ID NO: 4

SEQ ID NO: 18 - protein, 279 aa, Homo sapiens
MPTPLVHPHLPISSPRVSPFPPPAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 19 - protein, 185 aa, Homo sapiens
MGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 20 - protein, 185 aa, Homo sapiens; identical to SEQ ID NO: 19

SEQ ID NO: 21 - protein, 185 aa, Homo sapiens; identical to SEQ ID NO: 19

SEQ ID NO: 22 - protein, 458 aa, Homo sapiens; identical to SEQ ID NO: 4

SEQ ID NO: 23 - protein, 403 aa, Homo sapiens
MNRGVPFRHLLLVLQLGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 24 - skipped

SEQ ID NO: 25 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - skipped

SEQ ID NO: 27 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - skipped

SEQ ID NO: 29 - protein, 153 aa, Homo sapiens [sequence omitted]

SEQ ID NO: 30 - protein, 137 aa, Homo sapiens
MGLTSQLLPPLFFLLACAGNFVHGHKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 31 - protein, 136 aa, Homo sapiens
MGLTSQLLPPLFFLLACAGNFVHGHKCDITLQEIIKTLNSLTEQKTLCTELTVTDIFAASKQHACLPTSRHTGTICRPPSARVPSTSTRLQSPERTQLRRKPSAGLRLCSGSSTATMRRTLAAWVRLHSSSTGTSS

SEQ ID NO: 32 - skipped

SEQ ID NO: 33 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 34 - skipped

SEQ ID NO: 35 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 36 - skipped

SEQ ID NO: 37 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 38 - protein, 93 aa, Homo sapiens
MIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 39 - skipped

SEQ ID NO: 40 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 41 - skipped

SEQ ID NO: 42 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 43 - skipped

SEQ ID NO: 44 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 45 - skipped

SEQ ID NO: 46 - protein, 1721 aa, Homo sapiens
MLSATPLYGNVHSWMNSERVRMCGASEDRKILVNDGDASKARLELREENPLNHNVVDASTAHRIDGLAALSMDRTGLIREGLRVPGNIVYSSLCGLGSEKGREAATSTLGGLGFSSERNPEMQFKPNTPETVEASAVSGKPPNGFSAIYKTPPGIQKSAVATAEALGLDRPASDKQSPLNINGASYLRLPWVNPYMEGATPAIYPFLDSPNKYSLNMYKALLPQQSYSLAQPLYSPVCTNGERFLYLPPPHYVGPHIPSSLASPMRLSTPSASPAIPPLVHCADKSLPWKMGVSPGNPVDSHAYPHIQNSKQPRVPSAKAVTSGLPGDTALLLPPSPRPSPRVHLPTQPAADTYSEFHKHYARISTSPSVALSKPYMTVSSEFPAARLSNGKYPKAPEGGEGAQPVPGHARKTAVQDRKDGSSPPLLEKQTVTKDVTDKPLDLSSKVVDVDASKADHMKKMAPTVLVHSRAGSGLVLSGSEIPKETLSPPGNGCAIYRSEIISTAPSSWVVPGPSPNEENNGKSMSLKNKALDWAIPQQRSSSCPRMGGTDAVITNVSGSVSSAGRPASASPAPNANADGTKTSRSSVETTPSVIQHVGQPPATPAKHSSSTSSKGAKASNPEPSFKANENGLPPSSIFLSPNEAFRSPPIPYPRSYLPYPAPEGIAVSPLSLHGKGPVYPHPVLLPNGSLFPGHLAPKPGLPYGLPTGRPEFVTYQDALGLGMVHPMLIPHTPIEITKEEKPERRSRSHERARYEDPTLRNRFSEILETSSTKLHPDVPTDKNLKPNPNWNQGKTVVKSDKLVYVDLLREEPDAKTDTNVSKPSFAAESVGQSAEPPKPSVEPALQQHRDFIALREELGRISDFHETYTFKQPVFTVSKDSVLAGTNKENLGLPVSTPFLEPPLGSDGPAVTFGKTQEDPKPFCVGSAPPSVDVTPTYTKDGADEAESNDGKVLKPKPSKLAKRIANSAGYVGDRFKCVTTELYADSSQLSREQRALQMEGLQEDSILCLPAAYCERAMMRFSELEMKEREGGHPATKDSEMCKFSPADWERLKGNQDKKPKSVTLEEAIAEQNESERCEYSVGNKHRDPFEAPEDKDLPVEKYFVERQPVSEPPADQVASDMPHSPTLRVDRKRKVSGDSSHTETTAEEVPEDPLLKAKRRRVSKGLHPKKQRHLLHLRERWEQQVSAADGKPGRQSRKEVTQATQPEAIPQGTNITEEKPGRKRAEAKGNRSWSEESLKPSDNEQGLPVFSGSPPMKSLSSTSAGGKKQAQPSCAPASRPPAKQQKIKENQKTDVLCADEEEDCQAASLLQKYTDNSEKPSGKRLCKTKHLIPQESRRGLPLTGEYYVENADGKVTVRRFRKRPEPSSDYDLSPAKQEPKPFDRLQQLLPASQSTQLPCSSSPQETTQSRPMPPEARRLIVNKNAGETLLQRAARLGYEEVVLYCLENKICDVNHRDNAGYCALHEACARGWLNIVRHLLEYGADVNCSAQDGTRPLHDAVENDHLEIVRLLLSYGADPTLATYSGRTIMKMTHSELMEKFLTDYLNDLQGRNDDDASGTWDFYGSSVCEPDDESGYDVLANPPGPEDQDDDDDAYSDVFEFEFSETPLLPCYNIQVSVAQGPRNWLLLSDVLKKLKMSSRIFRCNFPNVEIVTIAEAEFYRQVSASLLFSCSKDLEAFNPESKELLDLVEFTNEIQTLLGSSVEWLHPSDLASDNYW

SEQ ID NO: 47 - protein, 1703 aa, Homo sapiens
MLSATPLYGNVHSWMNSERVRMCGASEDRKILVNDGDASKARLELREENPLNHNVVDASTAHRIDGLAALSMDRTGLIREGLRVPGNIVYSSLCGLGSEKGREAATSTLGGLGFSSERNPEMQFKPNTPETVEASAVSGKPPNGFSAIYKTPPGIQKSAVATAEALGLDRPASDKQSPLNINGASYLRLPWVNPYMEGATPAIYPFLDSPNKYSLNMYKALLPQQSYSLAQPLYSPVCTNGERFLYLPPPHYVGPHIPSSLASPMRLSTPSASPAIPPLVHCADKSLPWKMGVSPGNPVDSHAYPHIQNSKQPRVPSAKAVTSGLPGDTALLLPPSPRPSPRVHLPTQPAADTYSEFHKHYARISTSPSVALSKPYMTVSSEFPAARLSNGKYPKAPEGGEGAQPVPGHARKTAVQDRKDGSSPPLLEKQTVTKDVTDKPLDLSSKVVDVDASKADHMKKMAPTVLVHSRAGSGLVLSGSEIPKETLSPPGNGCAIYRSEIISTAPSSWVVPGPSPNEENNGKSMSLKNKALDWAIPQQRSSSCPRMGGTDAVITNVSGSVSSAGRPASASPAPNANADGTKTSRSSVETTPSVIQHVGQPPATPAKHSSSTSSKGAKASNPEPSFKANENGLPPSSIFLSPNEAFRSPPIPYPRSYLPYPAPEGIAVSPLSLHGKGPVYPHPVLLPNGSLFPGHLAPKPGLPYGLPTGRPEFVTYQDALGLGMVHPMLIPHTPIEITKEEKPERRSRSHERARYEDPTLRNRFSEILETSSTKLHPDVPTDKNLKPNPNWNQGKTVVKSDKLVYVDLLREEPDAKTDTNVSKPSFAAESVGQSAEPPKPSVEPALQQHRDFIALREELGRISDFHETYTFKQPVFTVSKDSVLAGTNKENLGLPVSTPFLEPPLGSDGPAVTFGKTQEDPKPFCVGSAPPSVDVTPTYTKDGADEAESNDGKVLKPKPSKLAKRIANSAGYVGDRFKCVTTELYADSSQLSREQRALQRAMMRFSELEMKEREGGHPATKDSEMCKFSPADWERLKGNQDKKPKSVTLEEAIAEQNESERCEYSVGNKHRDPFEAPEDKDLPVEKYFVERQPVSEPPADQVASDMPHSPTLRVDRKRKVSGDSSHTETTAEEVPEDPLLKAKRRRVSKGLHPKKQRHLLHLRERWEQQVSAADGKPGRQSRKEVTQATQPEAIPQGTNITEEKPGRKRAEAKGNRSWSEESLKPSDNEQGLPVFSGSPPMKSLSSTSAGGKKQAQPSCAPASRPPAKQQKIKENQKTDVLCADEEEDCQAASLLQKYTDNSEKPSGKRLCKTKHLIPQESRRGLPLTGEYYVENADGKVTVRRFRKRPEPSSDYDLSPAKQEPKPFDRLQQLLPASQSTQLPCSSSPQETTQSRPMPPEARRLIVNKNAGETLLQRAARLGYEEVVLYCLENKICDVNHRDNAGYCALHEACARGWLNIVRHLLEYGADVNCSAQDGTRPLHDAVENDHLEIVRLLLSYGADPTLATYSGRTIMKMTHSELMEKFLTDYLNDLQGRNDDDASGTWDFYGSSVCEPDDESGYDVLANPPGPEDQDDDDDAYSDVFEFEFSETPLLPCYNIQVSVAQGPRNWLLLSDVLKKLKMSSRIFRCNFPNVEIVTIAEAEFYRQVSASLLFSCSKDLEAFNPESKELLDLVEFTNEIQTLLGSSVEWLHPSDLASDNYW

SEQ ID NO: 48 - protein, 1755 aa, Homo sapiens
MLSATPLYGNVHSWMNSERVRMCGASEDRKILVNDGDASKARLELREENPLNHNVVDASTAHRIDGLAALSMDRTGLIREGLRVPGNIVYSSLCGLGSEKGREAATSTLGGLGFSSERNPEMQFKPNTPETVEASAVSGKPPNGFSAIYKTPPGIQKSAVATAEALGLDRPASDKQSPLNINGASYLRLPWVNPYMEGATPAIYPFLDSPNKYSLNMYKALLPQQSYSLAQPLYSPVCTNGERFLYLPPPHYVGPHIPSSLASPMRLSTPSASPAIPPLVHCADKSLPWKMGVSPGNPVDSHAYPHIQNSKQPRVPSAKAVTSGLPGDTALLLPPSPRPSPRVHLPTQPAADTYSEFHKHYARISTSPSVALSKPYMTVSSEFPAARLSNGKYPKAPEGGEGAQPVPGHARKTAVQDRKDGSSPPLLEKQTVTKDVTDKPLDLSSKVVDVDASKADHMKKMAPTVLVHSRAGSGLVLSGSEIPKETLSPPGNGCAIYRSEIISTAPSSWVVPGPSPNEENNGKSMSLKNKALDWAIPQQRSSSCPRMGGTDAVITNVSGSVSSAGRPASASPAPNANADGTKTSRSSVETTPSVIQHVGQPPATPAKHSSSTSSKGAKASNPEPSFKANENGLPPSSIFLSPNEAFRSPPIPYPRSYLPYPAPEGIAVSPLSLHGKGPVYPHPVLLPNGSLFPGHLAPKPGLPYGLPTGRPEFVTYQDALGLGMVHPMLIPHTPIEITKEEKPERRSRSHERARYEDPTLRNRFSEILETSSTKLHPDVPTDKNLKPNPNWNQGKTVVKSDKLVYVDLLREEPDAKTDTNVSKPSFAAESVGQSAEPPKPSVEPALQQHRDFIALREELGRISDFHETYTFKQPVFTVSKDSVLAGTNKENLGLPVSTPFLEPPLGSDGPAVTFGKTQEDPKPFCVGSAPPSVDVTPTYTKDGADEAESNDGKVLKPKPSKLAKRIANSAGYVGDRFKCVTTELYADSSQLSREQRALQMEGLQEDSILCLPAAYCERAMMRFSELEMKEREGGHPATKDSEMCKFSPADWERLKGNQDKKPKSVTLEEAIAEQNESERCEYSVGNKHRDPFEAPEDKDLPVEKYFVERQPVSEPPADQVASDMPHSPTLRVDRKRKVSGDSSHTETTAEEVPEDPLLKAKRRRVSKDDWPEREMTNSSSNHLEDPHYSELTNLKVCIELTGLHPKKQRHLLHLRERWEQQVSAADGKPGRQSRKEVTQATQPEAIPQGTNITEEKPGRKRAEAKGNRSWSEESLKPSDNEQGLPVFSGSPPMKSLSSTSAGGKKQAQPSCAPASRPPAKQQKIKENQKTDVLCADEEEDCQAASLLQKYTDNSEKPSGKRLCKTKHLIPQESRRGLPLTGEYYVENADGKVTVRRFRKRPEPSSDYDLSPAKQEPKPFDRLQQLLPASQSTQLPCSSSPQETTQSRPMPPEARRLIVNKNAGETLLQRAARLGYEEVVLYCLENKICDVNHRDNAGYCALHEACARGWLNIVRHLLEYGADVNCSAQDGTRPLHDAVENDHLEIVRLLLSYGADPTLATYSGRTIMKMTHSELMEKFLTDYLNDLQGRNDDDASGTWDFYGSSVCEPDDESGYDVLANPPGPEDQDDDDDAYSDVFEFEFSETPLLPCYNIQVSVAQGPRNWLLLSDVLKKLKMSSRIFRCNFPNVEIVTIAEAEFYRQVSASLLFSCSKDLEAFNPESKELLDLVEFTNEIQTLLGSSVEWLHPSDLASDNYW

SEQ ID NO: 49 - skipped

SEQ ID NO: 50 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 51 - skipped

SEQ ID NO: 52 - protein, 526 aa, Homo sapiens [sequence omitted]

SEQ ID NO: 53 - skipped

SEQ ID NO: 54 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 55 - protein, 415 aa, Homo sapiens
MRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 56 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 57 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 58 - protein, 164 aa, Homo sapiens; identical to SEQ ID NO: 25

SEQ ID NO: 59 - protein, 555 aa, Homo sapiens
MAARLLLLGILLLLLPLPVPAPCHTAARSECKRSHKFVPGAWLAGEGVDVTSLRRSGSFPVDTQRFLRPDGTCTLCENALQEGTLQRLPLALTNWRAQGSGCQRHVTRAKVSSTEAVARDAARSIRNDWKVGLDVTPKPTSNVHVSVAGSHSQAANFAAQKTHQDQYSFSTDTVECRFYSFHVVHTPPLHPDFKRALGDLPHHFNASTQPAYLRLISNYGTHFIRAVELGGRISALTALRTCELALEGLTDNEVEDCLTVEAQVNIGIHGSISAEAKACEEKKKKHKMTASFHQTYRERHSEVVGGHHTSINDLLFGIQAGPEQYSAWVNSLPGSPGLVDYTLEPLHVLLDSQDPRREALRRALSQYLTDRARWRDCSRPCPPGRQKSPRDPCQCVCHGSAVTTQDCCPRQRGLAQLEVTFIQAWGLWGDWFTATDAYVKLFFGGQELRTSTVWDNNNPIWSVRLDFGDVLLATGGPLRLQVWDQDSGRDDDLLGTCDQAPKSGSHEVRCNLNHGHLKFRYHARCLPHLGGGTCLDYVPQMLLGEPPGNRSGAVW

SEQ ID NO: 60 - protein, 235 aa, Homo sapiens; identical to SEQ ID NO: 3

SEQ ID NO: 61 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

(19) United States
(12) Patent Application Publication
(10) Pub. No.: US 2026/0242728 A1
(43) Pub. Date: Aug. 20, 2026

(54) CD4 + AND CD8 + SINGLE POSITIVE T CELLS AND COMPOSITIONS AND METHODS FOR GENERATING THE SAME

(71) Applicant: CLADE THERPAEUTICS, INC., Philadelphia, PA (US)

(72) Inventors: Dar Mikael HEINZE, Philadelphia, PA (US); Gordon Grant WELSTEAD, Philadelphia, PA (US)

(21) Appl. No.: 19/154,561

(22) PCT Filed: Feb. 8, 2024
(86) PCT No.: PCT/US2024/015017

§ 371 Date: Aug. 7, 2025

Related U.S. Application Data
(60) Provisional application No. 63/483,814, filed on Feb. 8, 2023.

(51) Int. Cl. C12N 5/0783; A61K 35/17; A61K 47/54; C07K 16/28

(52) U.S. Cl. CPC C12N 5/0636; A61K 35/17; A61K 47/557; C07K 16/2809; C07K 16/2812; C07K 16/2815; C12N 2506/45; C12N 2510/00

(57) ABSTRACT

Provided herein are cell and matrix compositions for the production of single positive T cells, such as CD4⁺ single positive T cells or CD8⁺ single positive T cells. Various methods for the generation of T cells by differentiation of stem cells are also provided. Further provided herein are methods of treating a disease or a condition in a subject, including, but not limited to cancer and autoimmune diseases.

### CROSS REFERENCE TO RELATED APPLICATIONS

This application claims the benefit of and priority to U.S. Provisional Patent Application No. 63/483,814 filed Feb. 8, 2023, the content of which is incorporated herein by reference in its entirety.

### BACKGROUND

There is a great unmet need in the field of cellular therapy to develop methods for scalable production of specific T cell subtypes for “off the shelf” therapies for the treatment of diseases such as cancer and autoimmune disease. One potential source for T cell therapies is stem cells that can be differentiated to lymphoid cells, including specific T cell subtypes that are involved in immunological memory, targeted effector function, and immune suppression. However, the generation of single positive CD4+ T cells and CD8+ T cells by differentiation of autologous or allogeneic stem cells has been challenging to date.

### BRIEF SUMMARY

Provided herein are compositions, wherein the compositions comprise: a matrix; a plurality of cell surface receptor binding agents, wherein the plurality of cell surface receptor binding agents are bound to the matrix and comprise: a CD4 receptor binding agent; a T cell receptor (TCR) binding agent; and a population of single positive CD4 T cells (CD4⁺ CD8⁻).

Further provided herein are compositions, wherein the compositions comprise: a matrix; a plurality of cell surface receptor binding agents, wherein the plurality of cell surface receptor binding agents are bound to the matrix and comprise: a CD4 receptor binding agent; and a T cell receptor (TCR) binding agent; and a mixed population of in vitro-differentiated T cells, wherein the mixed population of in vitro-differentiated T cells comprise: a population of single positive CD4⁺ T cells; and a population of double positive (CD4⁺ CD8⁺) T cells, wherein the population of single positive CD4⁺ T cells is at least about 10% of the total number of cells within the mixed population of in vitro-differentiated T cells.

Further provided herein are compositions, wherein the compositions comprise: a CD4 binding agent tethered to a TCR binding agent.

Further provided herein are compositions, wherein the compositions comprise: a CD4 binding agent fixed proximal to a TCR binding agent.

Further provided herein are compositions, wherein the compositions comprise: a matrix; a plurality of cell surface receptor binding agents, wherein the plurality of cell surface receptor binding agents are bound to the matrix and comprise: a CD8 receptor binding agent; a T cell receptor (TCR) binding agent; and a population of single positive CD8 T cells (CD4⁻ CD8⁺).

Further provided herein are compositions, wherein the compositions comprise: a matrix; a plurality of cell surface receptor binding agents, wherein the plurality of cell surface receptor binding agents are bound to the matrix and comprise: a CD8 receptor binding agent; and a T cell receptor (TCR) binding agent; and a mixed population of in vitro-differentiated T cells, wherein the mixed population of in vitro-differentiated T cells comprise: a population of single positive CD8⁺ T cells; and a population of double positive (CD4⁺ CD8⁺) T cells, wherein the population of single positive CD8⁺ T cells is at least about 10% of the total number of cells within the mixed population of in vitro-differentiated T cells.

Further provided herein are compositions, wherein the compositions comprise: a CD8 binding agent tethered to a TCR binding agent.

Further provided herein are compositions, wherein the compositions comprise: a CD8 binding agent fixed proximal to a TCR binding agent.

Further provided herein are fusion proteins, wherein the fusion proteins comprise: a CD4 binding agent that binds to SEQ ID NO: 1 or a functional fragment thereof; and a TCR binding agent that binds to SEQ ID NO: 2 or a functional fragment thereof.

Further provided herein are fusion proteins, wherein the fusion proteins comprise: a CD8 binding agent that binds to SEQ ID NO: 3 or a functional fragment thereof; and a TCR binding agent that binds to SEQ ID NO: 2 or a functional fragment thereof.

Further provided herein are in vitro-differentiated T cells, wherein the in vitro-differentiated T cells are bound to a composition provided herein; or the fusion protein provided herein.

Provided herein are methods of producing a population of single positive CD4+ T cells from a population of in vitro-differentiated double positive (CD4+CD8+) T cells, wherein the methods comprise: contacting a population of in vitro-differentiated (CD4+CD8+) T cells for a period of time with a composition comprising: (i) a matrix; (ii) a CD4 receptor binding agent that is bound to the matrix; and (iii) an a TCR binding agent that is bound to the matrix, thereby producing a population of single positive CD4+ T cells.

Provided herein are methods for producing a single positive CD4⁺ T cell from a double positive (CD4⁺ CD8⁻) T cell, wherein the methods comprise: contacting an in vitro-differentiated double positive (CD4⁺ CD8⁺) T cell with a CD4 binding agent tethered to a TCR binding agent.

Further provided herein are a population of single positive CD4⁺ T cells produced by the methods provided herein.

Provided herein are methods for producing a population of single positive CD8⁺ T cells from a population of in vitro-differentiated double positive (CD4⁺ CD8⁺) T cells, the method comprising: contacting a population of in vitro-differentiated CD4⁺ CD8⁺ T cells for a period of time with a composition comprising: (i) a matrix; (ii) a CD8 binding agent that is bound to the matrix; and (iii) a TCR binding agent that is bound to the matrix, thereby producing a population of single positive CD8⁺ T cells.

Provided herein are methods for producing a single positive CD8⁺ T cell from a double positive (CD4⁺ CD8⁻) T cell, wherein the methods comprise: contacting an in vitro-differentiated double positive (CD4⁺ CD8⁺) T cell with a CD8 binding agent tethered to a TCR binding agent.

Further provided herein are a population of single positive CD8⁺ T cells produced by the methods provided herein.

Provided herein are methods for the treatment of a disease or a condition in a subject, wherein the methods comprise: administering to a subject in need thereof a composition provided herein or a population of single positive CD4⁺ T cells provided herein, thereby treating the disease of condition.

Provided herein are systems for producing a single positive CD4⁺ T cell comprising: a cell culture vessel configured for localizing a plurality of cell surface receptor binding agents to an in vitro-differentiated double positive (CD4⁺ CD8⁺) T cell or a population thereof comprising; a matrix; a plurality of cell surface receptor binding agents, wherein the plurality of cell surface receptor binding agents are bound to the matrix and comprise: a CD4 receptor binding agent; a TCR binding agent; and an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time, and wherein the cell culture vessel is configured to facilitate contact between the plurality of cell surface receptor binding agents and the in vitro-differentiated double positive CD4⁺ CD8⁺ T cell or population thereof, thereby producing a single positive CD4⁺ T cell within at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days.

Provided herein is a system for producing a single positive CD8⁺ T cell comprising: a cell culture vessel configured localizing a plurality of cell surface receptor binding agents to an in vitro-differentiated double positive (CD4⁺ CD8⁺) T cell or a population thereof comprising; a matrix; a plurality of cell surface receptor binding agents, wherein the plurality of cell surface receptor binding agents are bound to the matrix and comprise: a CD8 receptor binding agent; a TCR binding agent; and an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time, and wherein the cell culture vessel is configured to facilitate contact between the plurality of cell surface receptor binding agents and the in vitro-differentiated double positive CD4⁺ CD8⁺ T cell or population thereof, thereby producing a single positive CD8⁺ T cell within at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days.

### BRIEF DESCRIPTION OF THE DRAWINGS

The novel features of the embodiments are set forth with particularity in the appended claims. A better understanding of the features and advantages of the present embodiments will be obtained by reference to the following detailed description that sets forth illustrative embodiments, in which the principles of the embodiments are utilized, and the accompanying drawings of which:

FIG. 1 shows a schematic for generating CD4⁺ and CD8⁺ single positive T cells starting from undifferentiated human pluripotent stem cells in accordance with the embodiments of the present disclosure. Stage 1 results in generation of CD34⁺ progenitor cells, that are further differentiated into CD4⁺ CD8⁺ double positive T cells (Stage 2), which can then be stimulated to generate both CD4⁺ single positive T cells and CD8⁺ singe positive cells (Stage 3) and matured and expanded (Stage 4).

FIG. 2A shows a schematic of an exemplary protocol used to generate single positive CD4⁺ T cells using a matrix-antibody stimulation system. Beads were complexed with anti-TCR alpha beta antibodies (anti-TCRαβ) and anti-CD4 receptor antibodies in a TCR and coreceptor stimulation medium. Medium factors and cell culture conditions are provided. FIG. 2B is a diagram illustrating an exemplary matrix and antibody composition complexed to a TCR and a CD4 coreceptor on the surface of a double positive (CD4⁺ CD8⁺) T cell.

FIG. 3A shows a schematic of an exemplary protocol used to generate single positive CD8⁺ T cells using a matrix-antibody stimulation system. Beads were complexed with anti-TCR alpha beta antibodies (anti-TCRαβ) and anti-CD8 receptor antibodies in a TCR and coreceptor stimulation medium. FIG. 3B is a diagram illustrating an exemplary matrix and antibody composition complexed to a TCR and a CD8 coreceptor on the surface of a double positive (CD4⁺ CD8⁺) T cell.

FIG. 4 shows a schematic of an exemplary protocol used to generate single positive CD4⁺ T cells and single positive CD8⁺ T cells from undifferentiated human pluripotent stem cells, including an optional restimulation step between Stage 3 and Stage 4.

FIG. 5 shows a schematic and exemplary flow cytometry data from differentiation of human induced pluripotent stem cells (iPSCs) into single positive, mature T cells under either CD4⁺ inducing conditions or CD8⁺ inducing conditions, and then subjected to a restimulation step with CD3/CD28/CD2 antibodies. At the end of the differentiation and restimulation, cell phenotype was assessed by flow cytometry (bottom right 4 panels).

FIG. 6A shows a graph of exemplary flow cytometry data for T cells produced after co-engagement of the TCR and a CD4 coreceptor. 50.3% of the population were single positive CD4⁺ T cells.

FIG. 6B shows a graph of exemplary flow cytometry data for T cells produced after co-engagement of the TCR and a CD8 coreceptor. 42.9% of the population were single positive CD8⁺ T cells.

FIG. 6C shows a graph of exemplary flow cytometry data for double positive T cells that were contacted with a bead and isotype antibodies as a negative control.

FIG. 7 shows exemplary data for expression of a CD4+ associated transcription factor (ThPOK) and a CD8+ associated transcription factor (RUNX3) in iPSC-derived T cells generated under CD4+SP-inducing or CD8+SP-inducing conditions, relative to control cells (CD4+ and CD8+ primary T cells).

FIG. 8 shows exemplary data from a cytotoxicity assay. The Stage 4, iPSC-derived SP T cells killed MM.1S tumor cells in the presence of anti-BCMA BITE, demonstrating successful generation of functional single positive T cells.

FIG. 9 shows exemplary data comparing the marker expression profiles of the iPSC-derived SP T cells of the present invention to the expression profiles of primary CD4+ and CD8+ T cells.

FIG. 10A shows a schematic of a tumor killing assay for comparing the anti-tumor activity of “bulk” iPSC-derived CD4⁺ T cells with a population of iPSC-derived CD4⁺ T cells enriched for CD4⁺ SP cells.

FIG. 10B shows exemplary data for anti-tumor activity of “bulk” iPSC-derived CD4⁺ T cells vs. a population of iPSC-derived CD4⁺ T cells enriched for CD4⁺ SP cells.

FIG. 11A shows the time points for transcriptional profiling and single cell RNA sequencing analysis subsequent to stimulating CD4⁺/CD8⁺ double positive (immature) cells under CD4⁺ SP-inducing conditions to generate mature iPSC-derived T cells.

FIG. 11B shows exemplary results of the transcriptional profiling analysis as outlined in FIG. 11A and as further described in Example 11.

Various aspects of the embodiments of the present disclosure now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION OF THE INVENTION

Provided herein are compositions, kits, methods, and uses thereof for the production of a population of in vitro-differentiated single positive CD4⁺ T cells or a population of in vitro-differentiated single positive CD8⁺ T cells. The T cells provided herein have multiple therapeutic applications and applications for research and development of new therapeutics. Briefly, further described herein are: (1) single positive CD4⁺ T cells and methods of producing the same; (2) single positive CD8⁺ T cells and methods of producing the same; (3) cell sources; (4) binding agent compositions; (5) matrix compositions; (6) pharmaceutical compositions; (7) dosing and administration; (8) therapeutic applications; (9) systems; and (10) kits.

While preferred embodiments of the present disclosure have been shown and described herein, such embodiments are provided by way of example only. Numerous variations, changes, and substitutions will occur to those skilled in the art without departing from the disclosure. It should be understood that various alternatives to the embodiments described herein may be employed. It is intended that the following claims define the scope of various embodiments and that methods and structures within the scope of these claims and their equivalents be covered thereby.

### Definitions

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, “optional” or “optionally” means that the subsequently described circumstance may or may not occur, so that the description includes instances where the circumstance occurs and instances where it does not.

Unless specifically stated or apparent from context, as used herein, the term “about” in reference to a number or range of numbers is understood to mean the stated number and numbers +/−20% thereof, or 20% below the lower listed limit and 20% above the higher listed limit for the values listed for a range.

The term “effective amount” or “therapeutically effective amount” refers to an amount that is sufficient to achieve or at least partially achieve the desired effect.

As used herein, the terms “engineered cell” and “genetically modified cell” refer to a cell that has undergone one more genetic modifications, resulting in, for example, reduced or abrogated expression of one more gene products that are normally expressed in such cells, or an increased or introduced expression of one more gene products that are not normally expressed (or are normally expressed at a lower level) in such cells. Without limiting the foregoing, exemplary engineered cells include human iPSCs and human iPSC-derived differentiated cells in which expression of certain HLA class I or class II molecules has been abrogated or “knocked out”, or which express engineered receptors such as CAR-Ts or gamma delta TCRs.

As used herein, the term “T cell” includes CAR-T cells, alpha beta T cells, gamma delta T cells, regulatory T cells, and NKT cells. The term “engineered T cell” means any T cell expressing any combination of naturally occurring or engineered receptors, with any combination of cellular enhancements.

### (1) Single Positive CD4⁺ T Cells and Methods of Producing the Same

Provided herein are compositions comprising a population of single positive CD4⁺ T cells. In some embodiments, the single positive CD4⁺ T cells are in vitro-differentiated single positive CD4⁺ T cells. In some embodiments, the single positive CD4⁺ T cells are differentiated from a population of stem cells. Exemplary stem cells include human pluripotent stem cells (such as induced pluripotent stem cells and human embryonic stem cells) and adult stem cells. In some embodiments, the single positive CD4⁺ T cells are derived from cells obtained from cord blood, placental cells, bone marrow, blood, or tissue biopsy. In some embodiments, the single positive CD4⁺ T cells are isolated from a mixed population of T cells. In some embodiments, the single positive CD4⁺ T cells are isolated from a population of peripheral blood mononuclear cells (PBMCs). Further provided herein are methods for producing a single positive CD4⁺ T cell and populations thereof from a stem cell. In some embodiments, the method comprises in vitro-differentiation and agent-mediated binding of a TCR on a double positive (CD4⁺ CD8⁺) T cell; and agent-mediated binding of a CD4 coreceptor or a CD8 coreceptor on the double positive (CD4⁺ CD8⁺) T cell. The differentiation of the different lineages of mammalian T cells from stem cells or T cell precursors depends on the network of specific cytokine signals, growth factors, transcription factors, and epigenetic modifications.

### In Vitro-Differentiation of Stem Cells to Produce CD34+ Progenitor Cells

Exemplary methods and compositions as provided herein are used to generate CD34+ progenitor cells by differentiation in vitro from stem cells. Exemplary stem cells used as a starting material for the embodiments of the present disclosure include but are not limited to human pluripotent stem cells (such as human embryonic stem cells and induced pluripotent stem cells) and human adult stem cells. Generally, throughout the differentiation process, a pluripotent cell will follow a developmental pathway along a particular developmental lineage, for example, the primary germ layers-ectoderm, mesoderm, or endoderm.

The embryonic germ layers are the source from which all tissues and organs derive in embryonic development. The mesoderm is the source of, for example, smooth and striated muscle, including cardiac muscle, connective tissue, vessels, the cardiovascular system, blood cells, bone marrow, skeleton, reproductive organs and excretory organs.

The germ layers can be identified by the expression of specific biomarkers and gene expression. Assays to detect these biomarkers include, for example, RT-PCR, immunohistochemistry, and Western blotting. Non-limiting examples of biomarkers expressed by early mesodermal cells include HAND1, ESM1, HAND2, HOPX, BMP10, FCN3, KDR, PDGFR-a, CD34, Tbx-6, Snail-1, Mesp-1, and GSC, among others. Biomarkers expressed by early ectoderm cells include but are not limited to TRPM8, POU4F1, OFFM3, WNT1, FMX1A and CDH9, among others. Biomarkers expressed by early endoderm cells include but are not limited to LEFTY1, EOMES, NODAF and FOXA2, among others. One of skill in the art can determine which lineage markers to monitor while performing a differentiation protocol based on the cell type and the germ layer from which that cell is derived in development.

Induction of a particular developmental lineage in vitro is accomplished by culturing stem cells in the presence of specific agents or combinations thereof that promote lineage commitment. Generally, the methods provided herein comprise the step-wise addition of agents (e.g., small molecules, growth factors, cytokines, polypeptides, vectors, etc.) into the cell culture medium or contacting a cell with agents that promote differentiation. In some embodiments, mesoderm formation is induced by transcription factors and growth factor signaling which includes but is not limited to vascular endothelial growth factor (VEGF), Wnt signaling (e.g., via beta-catenin), bone morphogenic protein (BMP) pathways, notch signaling, fibroblast growth factor (FGF) pathways, and transforming growth factor (TGF) signaling (e.g., activin A).

The differentiation process can be monitored for efficiency by a number of methods known in the art. This includes detecting the presence of germ layer biomarkers using standard techniques, for example, immunocytochemistry, RT-PCR, flow cytometry, functional assays, optical tracking, etc. The differentiation status of a cell is generally determined by one or more of characteristic gene or marker expression pattern, metabolic activities, and morphology.

### Stage 1:

Provided herein are methods of producing hematopoietic stem and progenitor cells, including CD34⁺ progenitor cells from a population of pluripotent stem cells. Further provided herein is a population of in vitro-differentiated CD34⁺ progenitor cells. In some embodiments, the methods comprise: (a) contacting a population of pluripotent stem cells with a first cell culture medium. Various types of cell culture media can be used to generate CD34⁺ progenitor cells and further differentiate such CD34⁺ progenitor cells to progenitor T cells and are discussed further below.

In some embodiments, the first cell culture medium comprises one or more growth factors. In some embodiments, the growth factor induces VEGF signaling, BMP signaling, notch signaling, FGF signaling, or TGF signaling in a cell relative to a comparable cell that has not been contacted with a first cell culture medium. In some embodiments, the first cell culture medium comprises a transforming growth factor β receptor family ligand. In some embodiments, the growth factor comprises BMP1, BMP2, BMP3, BMP4, BMP5, BMP6, BMP7, BMP8, BMP9, BMP10, fibroblast growth factor (FGF), epidermal growth factor (EGF), hedgehog molecules, insulin-like growth factor (IGF), platelet-derived growth factor (PDGF), VEGF, or a WNT molecule. In some embodiments, the first cell culture medium does not comprise VEGF.

In some embodiments, the first cell culture medium comprises a c-Kit receptor ligand. In some embodiments, the first cell culture medium comprises stem cell factor (SCF). In some embodiments, the first cell culture medium does not comprise stem cell factor (SCF). In some embodiments, the first cell culture medium does not comprise a Notch receptor ligand.

In some embodiments, the first cell culture medium comprises a serine-threonine kinase receptor ligand. In some embodiments, the first cell culture medium comprises a WNT pathway activator. In some embodiments, the WNT pathway activator comprises a glycogen synthase kinase 3 inhibitor. In some embodiments, the first cell culture medium comprises a glycogen synthase kinase 3 inhibitor. In some embodiments, the first cell culture medium does not comprise a glycogen synthase kinase 3 inhibitor. In some embodiments, the glycogen synthase kinase 3 inhibitor comprises a small molecule selected from the group consisting of: CHIR98014, CHIR98024, CHIR99021, 2,4′-dibromoacetophenone, and dihydronarwedine.

In some embodiments, the first cell culture medium comprises an activin receptor ligand. In some embodiments, the first cell culture medium does not comprise an activin receptor ligand. In some embodiments, the activin receptor ligand is a ligand of an ALK1 receptor, an ALK2 receptor, an ALK3 receptor, an ALK4 receptor, an ALK5 receptor, an ALK6 receptor, a ALK7 receptor, or any combination thereof. In some embodiments, the first cell culture medium comprises an anaplastic lymphoma kinase 5 (ALK5) inhibitor. In some embodiments, the first cell culture medium does not comprise an anaplastic lymphoma kinase 5 (ALK5) inhibitor. In some embodiments, the first cell culture medium comprises an anaplastic lymphoma kinase 6 (ALK6) inhibitor. In some embodiments, the first cell culture medium does not comprise an anaplastic lymphoma kinase 6 (ALK6) inhibitor. In some embodiments, the first cell culture medium comprises an anaplastic lymphoma kinase 7 (ALK7) inhibitor. In some embodiments, the first cell culture medium does not comprise an anaplastic lymphoma kinase 7 (ALK7) inhibitor. In some embodiments, the first cell culture medium comprises a Rho kinase (ROCK) inhibitor. In some embodiments, the first cell culture medium does not comprise a ROCK inhibitor. In some embodiments, the Rho kinase (ROCK) inhibitor is selective for ROCK1. In some embodiments, the ROCK inhibitor comprises thiazovivin, Y-27632, Y-30141, Y-33075, Y-39983, or any combination thereof. In some embodiments, the first cell culture medium does not comprise thyroid peroxidase. In some embodiments, the first cell culture medium comprises thyroid peroxidase. In some embodiments, the first cell culture medium comprises a CD135 receptor ligand. In some embodiments, the first cell culture medium does not comprise a CD135 receptor ligand. In some embodiment, the CD135 receptor ligand is FMS-like tyrosine kinase 3 ligand (FLT3L, NCBI Gene ID: 2323), an FLT3L fusion protein. In some embodiments, the first cell culture medium comprises a CD135 receptor inhibitor. In some embodiments, the first cell culture medium does not comprise a CD135 receptor inhibitor. In some embodiments, the CD135 receptor inhibitor comprises gilteritinib, lestaurtinib, midostaurin, sorafenib, or sunitinib.

In some embodiments, the first cell culture medium comprises one or more organosulfur compound. Organosulfur compounds are organic compounds that contain sulfur. In some embodiments, the organosulfur compound is allicin, diphenyl disulfide, dibenzothiophene, dimethyl sulfoxide (DMSO), lipoic acid, methionine, penicillin, sulfanilamide. In some embodiments, the first cell culture medium comprises dihydrolevoglucosenone. In some embodiments, the first cell culture medium comprises an amino acid. Exemplary amino acids include but are not limited to alanine, arginine, cysteine, glutamine, glutamate, histidine, leucine, lysine, methionine, phenylalanine, serine, tryptophan, or tyrosine.

In some embodiments, the first cell culture medium comprises one or more cytokine. Exemplary cytokines include but are not limited to: granulocyte-macrophage colony-stimulating factor (GMCSF), IL-2, IL-3, IL-4, IL-6, IL-7, IL-11, IL-15, IL-21, and tumor necrosis factor alpha (TNFα). In some embodiments, the first cell culture medium comprises a thrombopoietin receptor ligand. In some embodiments, the first cell culture medium comprises thrombopoietin (TPO). In some embodiments, the first cell culture medium does not comprise retinoic acid. In some embodiments, the first cell culture medium does not comprise retinoic acid.

In some embodiments, the methods further comprise culturing the population of pluripotent stem cells are co-cultured with another cell type. In some embodiments, the population of pluripotent stem cells are co-cultured with stromal cells or mouse embryonic fibroblasts. In some embodiments, the population of pluripotent stem cells are not co-cultured with stromal cells or mouse embryonic fibroblasts. In some embodiments, the stromal cells comprise OP9 stromal cells.

In some embodiments, pluripotent stem cells are cultured as aggregates. In some embodiments, the methods comprise culturing the population of pluripotent stem cells in or on the surface of a moving apparatus. In some embodiments, the moving apparatus is an orbital shaker. In some embodiments, the population of pluripotent stem cells are continuously rotated to form cell aggregates. In some embodiments, the continuous rotation is performed for at least about 12 hours, 24 hours, up to 48 hours. In some embodiments, the continuous rotation is performed at a rotation per minute of at least 90 rpm, 95 rpm, 100 rpm, 105 rpm, 110 rpm, 115 rpm, 120 rpm, 125 rpm, up to about 150 rpm. In some embodiments, the method further comprises culturing the population of pluripotent stem cells in a flow chamber. In some embodiments, the population of pluripotent stem cells are cultured in laminar flow conditions. In some embodiments, the laminar flow rate is at least about 100 microliters per minute (μL/min), about 125 μL/min, about 150 μL/min, about 175 μL/min, about 200 μL/min, about 225 μL/min, about 250 μL/min, about 275 μL/min, about 300 μL/min, about 425 μL/min, about 450 μL/min, about 475 μL/min, about 500 μL/min, about 525 μL/min, about 550 μL/min, about 575 μL/min, about 600 μL/min, about 625 μL/min, about 650 μL/min, about 675 μL/min, about 700 μL/min, about 725 μL/min, about 750 μL/min, about 775 μL/min, up to 800 μL/min. In some embodiments, the population of pluripotent stem cells are cultured in static conditions. In some embodiments, the culturing is performed for at least about 12 hours, 24 hours, up to 48 hours.

In some embodiments, the population of pluripotent stem cells are cultured in 2-dimensional (2D) culture. In some embodiments, the population of pluripotent stem cells are cultured in 3-dimensional (3D) culture. In some embodiments, the population of pluripotent stem cells are cultured in hypoxic conditions. In some embodiments, the population of pluripotent stem cells are cultured in at least about 0.5% (v/v) oxygen or more, 1% (v/v) oxygen or more, 5% (v/v) oxygen or more, 10% (v/v) oxygen or more, 15% (v/v) oxygen or more, up to 18.5% (v/v) oxygen. In some embodiments, the population of pluripotent stem cells are cultured as a monolayer on a 2D culture, and the subsequent differentiation steps are performed in a 3D culture. In other embodiments, the population of pluripotent stem cells are cultured as a monolayer on a 2D culture, and the subsequent differentiation steps are performed in a 3D culture, in a 2D culture, or in a combination of 3D culture and 2D culture steps. In some embodiments, the population of pluripotent stem cells are aggregated before performing subsequent differentiation steps in a 3D culture.

In some embodiments, the population of pluripotent stem cells are dissociated into a plurality of populations of pluripotent stem cells using a solution of proteolytic and collagenolytic enzymes. In some embodiments, the population of pluripotent stem cells are dissociated using trypsin-ethylenediaminetetraacetic acid (EDTA). Any methods known in the art may be used to dissociate pluripotent stem cells cultured as aggregates or under adherent monolayer culture conditions.

In some embodiments, the population of pluripotent stem cells are contacted with the first cell culture medium for at least about 12 hours, 24 hours, 36 hours, 48 hours, 60 hours, up to 72 hours. In some embodiments, the population of pluripotent stem cells are contacted with the first cell culture medium for at least about 24 hours. In some embodiments, the methods further comprise culturing the population of stem cells for a period of time in the first cell culture medium, thereby producing a population of early hematopoietic progenitor cells. In some embodiments, the period of time is at least about 12 hours, 24 hours, 36 hours, 48 hours, 60 hours, or 72 hours. In some embodiments, the period of time is at least about 1 day, 2 days, 3 days, 4 days, 5 days, 6 days, or 7 days.

In some embodiments, the method further comprises, removing the first cell culture medium and contacting the cells with a second cell culture medium. In some embodiments, the methods further comprise culturing the population of cells for a period of time in the second cell culture medium, thereby producing a population of early hematopoietic progenitor cells. In some embodiments, the period of time is at least about 12 hours, 24 hours, 36 hours, 48 hours, 60 hours, or 72 hours. In some embodiments, the period of time is at least about 1 day, 2 days, 3 days, 4 days, 5 days, 6 days, up to 7 days.

In some embodiments, the second cell culture medium comprises a phosphoinositol 3 kinase (PI3K) inhibitor. In some embodiments, the second cell culture medium comprises a deoxyribonucleic acid protein kinase (DNA-PK) inhibitor. In some embodiments, the second cell culture medium comprises with a deoxyribonucleic acid protein kinase (DNA-PK) inhibitor and a phosphoinositol 3 kinase (PI3K) inhibitor. In some embodiments, the second cell culture medium does not comprise a deoxyribonucleic acid protein kinase (DNA-PK) inhibitor or a phosphoinositol 3 kinase (PI3K) inhibitor. In some embodiments, the second cell culture medium does not comprise a deoxyribonucleic acid protein kinase (DNA-PK) inhibitor and a phosphoinositol 3 kinase (PI3K) inhibitor. In some embodiments, the PI3K inhibitor is selected from the group consisting of: BAY-1082439, CNX-1351, GNE-317, PQR530, MDK34597, phenylarsine oxide, PIK-90, TG100713, TGR-1202, and voxtalisib.

In some embodiments, the second cell culture medium comprises one or more growth factors. In some embodiments, the growth factor induces VEGF signaling, BMP signaling, notch signaling, FGF signaling, or TGF signaling in a cell relative to a comparable cell that has not been contacted with the second cell culture medium. In some embodiments, the second cell culture medium comprises a transforming growth factor β receptor family ligand. In some embodiments, the growth factor comprises BMP1, BMP2, BMP3, BMP4, BMP5, BMP6, BMP7, BMP8, BMP9, BMP10, fibroblast growth factor (FGF), epidermal growth factor (EGF), hedgehog molecules, insulin-like growth factor (IGF), platelet-derived growth factor (PDGF), VEGF, or a WNT molecule. In some embodiments, the second cell culture medium does not comprise VEGF. In some embodiments, the second cell culture medium comprises a WNT pathway activator. In some embodiments, the WNT pathway activator comprises a glycogen synthase kinase 3 inhibitor. In some embodiments, the second cell culture medium does not comprise a glycogen synthase kinase 3 inhibitor. In some embodiments, the glycogen synthase kinase 3 inhibitor comprises a small molecule selected from the group consisting of: CHIR98014, CHIR98024, CHIR99021, 2,4′-dibromoacetophenone, and dihydronarwedine.

In some embodiments, the second cell culture medium comprises an activin receptor ligand. In some embodiments, the second cell culture medium does not comprise an activin receptor ligand. In some embodiments, the second cell culture medium comprises A-77-01, EW-7197, GW 788388, LDN-193189, LDN-214117, SB-431542, SB-505124, or SM-16. In some embodiments, the second cell culture medium comprises a ROCK inhibitor. In some embodiments, the second cell culture medium does not comprise a ROCK inhibitor.

In some embodiments, the second cell culture medium comprises one or more organosulfur compound. Organosulfur compounds are organic compounds that contain sulfur. In some embodiments, the organosulfur compound is allicin, diphenyl disulfide, dibenzothiophene, dimethyl sulfoxide (DMSO), lipoic acid, methionine, penicillin, sulfanilamide. In some embodiments, the second cell culture medium comprises dihydrolevoglucosenone. In some embodiments, the second cell culture medium comprises an amino acid. Exemplary amino acids include but are not limited to alanine, arginine, cysteine, glutamine, glutamate, histidine, leucine, lysine, methionine, phenylalanine, serine, tryptophan, or tyrosine.

In some embodiments, the second cell culture medium comprises one or more cytokine. In some embodiments, the second cell culture medium comprises a thrombopoietin receptor ligand. In some embodiments, the second cell culture medium does not comprise thrombopoietin (TPO). In some embodiments, the second cell culture medium comprises retinoic acid. In some embodiments, the second cell culture medium does not comprise retinoic acid.

In some embodiments, the methods further comprise culturing the population of pluripotent stem cells are co-cultured with another cell type. In some embodiments, the population of pluripotent stem cells are co-cultured with stromal cells or mouse embryonic fibroblasts. In some embodiments, the population of pluripotent stem cells are not co-cultured with stromal cells or mouse embryonic fibroblasts. In some embodiments, the stromal cells comprise OP9 stromal cells.

In some embodiments, the methods further comprise culturing the population of stem cells or their differentiated progeny in or on the surface of a moving apparatus. In some embodiments, the moving apparatus is an orbital shaker. In some embodiments, the population of mesodermal stem cells are continuously rotated to form cell aggregates. In some embodiments, the continuous rotation is performed for at least about 12 hours, 24 hours, up to 48 hours. In some embodiments, the continuous rotation is performed at a rotation per minute of at least 90 rpm, 95 rpm, 100 rpm, 105 rpm, 110 rpm, 115 rpm, 120 rpm, 125 rpm, up to about 150 rpm. In some embodiments, the method further comprises culturing the population of mesodermal stem cells in a flow chamber. In some embodiments, the population of mesodermal stem cells are cultured in laminar flow conditions. In some embodiments, the laminar flow rate is at least about 100 microliters per minute (μL/min), about 125 μL/min, about 150 μL/min, about 175 μL/min, about 200 μL/min, about 225 μL/min, about 250 μL/min, about 275 μL/min, about 300 μL/min, about 425 μL/min, about 450 μL/min, about 475 μL/min, about 500 μL/min, about 525 μL/min, about 550 μL/min, about 575 μL/min, about 600 μL/min, about 625 μL/min, about 650 μL/min, about 675 μL/min, about 700 μL/min, about 725 μL/min, about 750 μL/min, about 775 μL/min, up to 800 μL/min. In some embodiments, the population of mesodermal stem cells are cultured in static conditions. In some embodiments, the culturing is performed for at least about 12 hours, 24 hours, up to 48 hours.

In some embodiments, the population of stem cells or their differentiated progeny are cultured in 2-dimensional (2D) culture. In some embodiments, the population of stem cells or their differentiated progeny are cultured in 3-dimensional (3D) culture. In some embodiments, the population of stem cells or their differentiated progeny are cultured in hypoxic conditions. In some embodiments, the population of stem cells or their differentiated progeny are cultured in at least about 0.5% (v/v) oxygen or more, 1% (v/v) oxygen or more, 5% (v/v) oxygen or more, 10% (v/v) oxygen or more, 15% (v/v) oxygen or more, up to 18.5% (v/v) oxygen.

In some embodiments, the contacting of the population of stem cells with the second culture medium produces a population of early hematopoietic progenitor cells. In some embodiments, the population of stem cells are contacted with the second cell culture medium for at least about 12 hours, 24 hours, 36 hours, 48 hours, 60 hours, up to 72 hours. In some embodiments, the population of stem cells are contacted with the second cell culture medium for at least about 24 hours. In some embodiments, the population of stem cells are contacted with the second cell culture medium for at least about 48 hours. In some embodiments, the methods further comprise culturing the population of stem cells for a period of time in the second cell culture medium, thereby producing a population of early hematopoietic progenitor cells. In some embodiments, the period of time is at least about 12 hours, 24 hours, 36 hours, 48 hours, 60 hours, or 72 hours. In some embodiments, the period of time is at least about 1 day, 2 days, 3 days, 4 days, 5 days, 6 days, up to 7 days.

Provided herein is a population of early hematopoietic progenitor cells made by the methods provided herein. In some embodiments, the population of early hematopoietic progenitor cells comprise a population of CD34⁺ progenitor cells.

Further provided herein is a method of producing a population of CD34⁺ progenitor cells from a population of early hematopoietic progenitor cells, the method comprising: (a) contacting a population of early hematopoietic progenitor cells with a third cell culture medium, thereby producing a population of CD34⁺ hematopoietic progenitor cells.

In some embodiments, the methods comprise removing the second cell culture medium and contacting the population of early hematopoietic progenitor cells with a third cell culture medium. In some embodiments, the third cell culture medium comprises one or more growth factors. In some embodiments, the third cell culture medium comprises a transforming growth factor β receptor family ligand. In some embodiments, the third cell culture medium comprises a serine-threonine kinase receptor ligand. In some embodiments, the third cell culture medium comprises a glycogen synthase kinase 3 inhibitor. In some embodiments, the third cell culture medium does not comprise a glycogen synthase kinase 3 inhibitor. In some embodiments, the third cell culture medium comprises an activin receptor ligand. In some embodiments, the third cell culture medium does not comprise an activin receptor ligand. In some embodiments, the activin receptor ligand is a ligand of an ALK1 receptor, an ALK2 receptor, an ALK3 receptor, an ALK4 receptor, an ALK5 receptor, an ALK6 receptor, a ALK7 receptor, or any combination thereof. In some embodiments, the third cell culture medium comprises an anaplastic lymphoma kinase 5 (ALK5) inhibitor. In some embodiments, the third cell culture medium does not comprise an anaplastic lymphoma kinase 5 (ALK5) inhibitor. In some embodiments, the third cell culture medium comprises an anaplastic lymphoma kinase 6 (ALK6) inhibitor. In some embodiments, the third cell culture medium does not comprise an anaplastic lymphoma kinase 6 (ALK6) inhibitor. In some embodiments, the third cell culture medium comprises an anaplastic lymphoma kinase 7 (ALK7) inhibitor. In some embodiments, the third cell culture medium does not comprise an anaplastic lymphoma kinase 7 (ALK7) inhibitor. In some embodiments, the third cell culture medium comprises a Rho kinase (ROCK) inhibitor. In some embodiments, the third cell culture medium does not comprise a ROCK inhibitor. In some embodiments, the Rho kinase (ROCK) inhibitor is selective for ROCK1. In some embodiments, the ROCK inhibitor comprises thiazovivin, Y-27632, Y-30141, Y-33075, Y-39983, or any combination thereof. In some embodiments, the third cell culture medium does not comprise thyroid peroxidase. In some embodiments, the third cell culture medium comprises thyroid peroxidase. In some embodiments, the third cell culture medium comprises a CD135 receptor ligand. In some embodiments, the third cell culture medium does not comprise a CD135 receptor ligand. In some embodiment, the CD135 receptor ligand is FMS-like tyrosine kinase 3 ligand (FLT3L, NCBI Gene ID: 2323), an FLT3L fusion protein. In some embodiments, the third cell culture medium comprises a CD135 receptor inhibitor. In some embodiments, the third cell culture medium does not comprise a CD135 receptor inhibitor. In some embodiments, the CD135 receptor inhibitor comprises gilteritinib, lestaurtinib, midostaurin, sorafenib, or sunitinib.

In some embodiments, the third cell culture medium comprises one or more organosulfur compound. Organosulfur compounds are organic compounds that contain sulfur. In some embodiments, the organosulfur compound is allicin, diphenyl disulfide, dibenzothiophene, dimethyl sulfoxide (DMSO), lipoic acid, methionine, penicillin, sulfanilamide. In some embodiments, the third cell culture medium comprises dihydrolevoglucosenone. In some embodiments, the third cell culture medium comprises an amino acid. Exemplary amino acids include but are not limited to alanine, arginine, cysteine, glutamine, glutamate, histidine, leucine, lysine, methionine, phenylalanine, serine, tryptophan, or tyrosine.

In some embodiments, the third cell culture medium comprises one or more cytokine. Exemplary cytokines include but are not limited to: granulocyte-macrophage colony-stimulating factor (GMCSF), IL-2, IL-3, IL-4, IL-6, IL-7, IL-11, IL-15, IL-21, and tumor necrosis factor alpha (TNFα). In some embodiments, the third cell culture medium comprises a thrombopoietin receptor ligand. In some embodiments, the third cell culture medium does not comprise thrombopoietin (TPO). In some embodiments, the third cell culture medium comprises retinoic acid. In some embodiments, the third cell culture medium does not comprise retinoic acid.

In some embodiments, the methods further comprise culturing the population of early hematopoietic stem cells are co-cultured with another cell type. In some embodiments, the population of early hematopoietic stem cells are co-cultured with stromal cells or mouse embryonic fibroblasts. In some embodiments, the population of early hematopoietic stem cells are not co-cultured with stromal cells or mouse embryonic fibroblasts. In some embodiments, the stromal cells comprise OP9 stromal cells.

In some embodiments, the methods further comprise culturing the population of early hematopoietic stem cells in or on the surface of a moving apparatus. In some embodiments, the population of early hematopoietic progenitor cells are continuously rotated to form cell aggregates. In some embodiments, the continuous rotation is performed for at least about 12 hours, 24 hours, up to 48 hours. In some embodiments, the continuous rotation is performed at a rotation per minute of at least 90 rpm, 95 rpm, 100 rpm, 105 rpm, 110 rpm, 115 rpm, 120 rpm, 125 rpm, up to about 150 rpm. In some embodiments, the method further comprises culturing the population of early hematopoietic progenitor cells in a flow chamber. In some embodiments, the population of early hematopoietic progenitor cells are cultured in laminar flow conditions. In some embodiments, the laminar flow rate is at least about 100 microliters per minute (μL/min), about 125 μL/min, about 150 μL/min, about 175 μL/min, about 200 μL/min, about 225 μL/min, about 250 μL/min, about 275 μL/min, about 300 L/min, about 425 μL/min, about 450 μL/min, about 475 μL/min, about 500 μL/min, about 525 L/min, about 550 μL/min, about 575 μL/min, about 600 μL/min, about 625 μL/min, about 650 μL/min, about 675 μL/min, about 700 μL/min, about 725 μL/min, about 750 μL/min, about 775 μL/min, up to 800 μL/min. In some embodiments, the population of early hematopoietic progenitor cells are cultured in static conditions. In some embodiments, the culturing is for at least about 12 hours, 36 hours, 48 hours, 60 hours, 72 hours, 96 hours, 108 hours, 120 hours, 132 hours, 144 hours, 156 hours, up to 168 hours.

In some embodiments, the population of early hematopoietic stem cells are cultured in 2-dimensional (2D) culture. In some embodiments, the population of early hematopoietic stem cells are cultured in 3-dimensional (3D) culture.

In some embodiments, the population of early hematopoietic stem cells are dissociated using a solution of proteolytic and collagenolytic enzymes. In some embodiments, the population of early hematopoietic stem cells are dissociated using trypsin-EDTA.

In some embodiments, the contacting of the population of early hematopoietic stem cells with the third culture medium produces a population of CD34⁺ progenitor cells. In some embodiments, the population of early hematopoietic stem cells are contacted with the third cell culture medium for at least about 12 hours, 24 hours, 36 hours, 48 hours, 60 hours, or 72 hours In some embodiments, the population of early hematopoietic stem cells are contacted with the third cell culture medium for at least about 24 hours. In some embodiments, the population of early hematopoietic stem cells are contacted with the third cell culture medium for at least about 48 hours. In some embodiments, the contacting is at least about 1 day, 2 days, 3 days, 4 days, 5 days, 6 days, up to 7 days. In some embodiments, the methods further comprise culturing the population of cells for a period of time in the third cell culture medium, thereby producing a population of CD34⁺ hematopoietic progenitor cells. In some embodiments, the period of time is at least about 12 hours, 24 hours, 36 hours, 48 hours, 60 hours, or 72 hours. In some embodiments, the period of time is at least about 1 day, 2 days, 3 days, 4 days, 5 days, 6 days, up to 7 days. In some embodiments, the method further comprises enriching the population of cells for CD34⁺ hematopoietic progenitor cells.

Further provided herein is a population of CD34 progenitor cells produced by the methods provided herein.

In some embodiments, the population of CD34⁺ progenitor cells are further dissociated with a mixture of proteolytic and collagenolytic enzymes (e.g., Accutase™). In some embodiments, the CD34⁺ progenitor cells are dissociated using trypsin-EDTA. In some embodiments, the dissociated CD34⁺ progenitor cells are replated into a cell culture vessel or onto an ECM composition. In some embodiments, the ECM composition comprises fibronectin, retronectin, laminin, vitronectin, human Fc-delta-like protein 4, solubilized basement membrane preparation extracted from the Engelbreth-Holm-Swarm (EHS) mouse sarcoma, methylcellulose, collagen, elastin, hyaluronic acid, or any combination thereof.

In some embodiments, the CD34⁺ progenitor cells are further strained in a cell strainer. In some embodiments, the CD34⁺ progenitor cells are enriched by flow cytometry, centrifugation, magnetic sorting, or a cell capture method. In some embodiments, the CD34⁺ progenitor cells are sorted from a mixed population of hematopoietic progenitor cells and pluripotent stem cells by flow cytometry.

### Stage 1 to Stage 2a

Further provided herein are methods of producing a population of T cell progenitor cells from a population of CD34⁺ progenitor cells. In some embodiments, the methods comprise contacting the population of CD34⁺ hematopoietic progenitor cells with a fourth cell culture medium.

In some embodiments, the fourth cell culture medium comprises albumin, insulin transferrin, or an antioxidant. In some embodiments, the fourth cell culture medium comprises albumin, insulin transferrin, and an antioxidant. In some embodiments, the fourth cell culture medium comprises one or more growth factors. In some embodiments, the growth factor is insulin-like growth factor. In some embodiments, the fourth cell culture medium comprises a transforming growth factor β receptor family ligand. In some embodiments, the fourth cell culture medium comprises a serine-threonine kinase receptor ligand. In some embodiments, the fourth cell culture medium comprises a glycogen synthase kinase 3 inhibitor. In some embodiments, the fourth cell culture medium does not comprise a glycogen synthase kinase 3 inhibitor. In some embodiments, the fourth cell culture medium comprises an activin receptor ligand. In some embodiments, the fourth cell culture medium does not comprise an activin receptor ligand. In some embodiments, the activin receptor ligand is a ligand of an ALK1 receptor, an ALK2 receptor, an ALK3 receptor, an ALK4 receptor, an ALK5 receptor, an ALK6 receptor, a ALK7 receptor, or any combination thereof. In some embodiments, the fourth cell culture medium comprises an anaplastic lymphoma kinase 5 (ALK5) inhibitor. In some embodiments, the fourth cell culture medium does not comprise an anaplastic lymphoma kinase 5 (ALK5) inhibitor. In some embodiments, the fourth cell culture medium comprises an anaplastic lymphoma kinase 6 (ALK6) inhibitor. In some embodiments, the fourth cell culture medium does not comprise an anaplastic lymphoma kinase 6 (ALK6) inhibitor. In some embodiments, the fourth cell culture medium comprises an anaplastic lymphoma kinase 7 (ALK7) inhibitor. In some embodiments, the fourth cell culture medium does not comprise an anaplastic lymphoma kinase 7 (ALK7) inhibitor. In some embodiments, the fourth cell culture medium comprises a Rho kinase (ROCK) inhibitor. In some embodiments, the fourth cell culture medium does not comprise a ROCK inhibitor. In some embodiments, the Rho kinase (ROCK) inhibitor is selective for ROCK1. In some embodiments, the ROCK inhibitor comprises thiazovivin, Y-27632, Y-30141, Y-33075, Y-39983, or any combination thereof. In some embodiments, the fourth cell culture medium does not comprise thyroid peroxidase. In some embodiments, the fourth cell culture medium comprises thyroid peroxidase. In some embodiments, the fourth cell culture medium comprises a CD135 receptor ligand. In some embodiments, the fourth cell culture medium does not comprise a CD135 receptor ligand. In some embodiment, the CD135 receptor ligand is FMS-like tyrosine kinase 3 ligand (FLT3L, NCBI Gene ID: 2323), an FLT3L fusion protein. In some embodiments, the fourth cell culture medium comprises a CD135 receptor inhibitor. In some embodiments, the fourth cell culture medium does not comprise a CD135 receptor inhibitor. In some embodiments, the CD135 receptor inhibitor comprises gilteritinib, lestaurtinib, midostaurin, sorafenib, or sunitinib.

In some embodiments, the fourth cell culture medium comprises one or more organosulfur compound. Organosulfur compounds are organic compounds that contain sulfur. In some embodiments, the organosulfur compound is allicin, diphenyl disulfide, dibenzothiophene, dimethyl sulfoxide (DMSO), lipoic acid, methionine, penicillin, sulfanilamide. In some embodiments, the fourth cell culture medium comprises dihydrolevoglucosenone. In some embodiments, the fourth cell culture medium comprises an amino acid. Exemplary amino acids include but are not limited to alanine, arginine, cysteine, glutamine, glutamate, histidine, leucine, lysine, methionine, phenylalanine, serine, tryptophan, or tyrosine.

In some embodiments, the fourth cell culture medium comprises one or more cytokine. Exemplary cytokines include but are not limited to: granulocyte-macrophage colony-stimulating factor (GMCSF), IL-2, IL-3, IL-4, IL-6, IL-7, IL-11, IL-15, IL-21, and tumor necrosis factor alpha (TNFα). In some embodiments, the fourth cell culture medium comprises a thrombopoietin receptor ligand. In some embodiments, the fourth cell culture medium does not comprise thrombopoietin (TPO). In some embodiments, the fourth cell culture medium comprises retinoic acid. In some embodiments, the fourth cell culture medium does not comprise retinoic acid.

In some embodiments, the population of CD34⁺ progenitor cells are contacted with the fourth cell culture medium for at least about 12 hours, 36 hours, 48 hours, 60 hours, 72 hours, 96 hours, 108 hours, 120 hours, 132 hours, 144 hours, 156 hours, or 168 hours. In some embodiments, the population of CD34⁺ progenitor cells are contacted with the fourth cell culture medium for at least about 1 day, 2 days, 3 days, 4 days, 5 days, 6 days, 7 days, 14 days, 21 days, 28 days, up to 35 days.

In some embodiments, the methods further comprise culturing the population of CD34⁺ progenitor cells in or on the surface of a moving apparatus. In some embodiments, the population of CD34⁺ hematopoietic progenitor cells are cultured in conditions that permit circular rotation. In some embodiments, the circular rotation is at least about 90 rpm, 95 rpm, 100 rpm, 105 rpm, 110 rpm, 115 rpm, 120 rpm, 125 rpm, up to 150 rpm. In some embodiments, the population of CD34⁺ progenitor cells are cultured in laminar flow conditions. In some embodiments, the laminar flow rate is at least about 100 microliters per minute (μL/min), about 125 μL/min, about 150 μL/min, about 175 μL/min, about 200 μL/min, about 225 μL/min, about 250 μL/min, about 275 μL/min, about 300 μL/min, about 425 μL/min, about 450 μL/min, about 475 μL/min, about 500 μL/min, about 525 μL/min, about 550 μL/min, about 575 μL/min, about 600 μL/min, about 625 μL/min, about 650 μL/min, about 675 μL/min, about 700 μL/min, about 725 μL/min, about 750 μL/min, about 775 μL/min, up to 800 μL/min. In some embodiments, the population of CD34⁺ progenitor cells are cultured in static conditions. In some embodiments, the culturing is performed for at least about 12 hours, 36 hours, 48 hours, 60 hours, 72 hours, 96 hours, 108 hours, 120 hours, 132 hours, 144 hours, 156 hours, or 168 hours up to 21 days. In some embodiments, the culturing is for at least about 1 day, 7 days, 14 days, 21 days, 28 days, up to 35 days.

In some embodiments, the population of CD34⁺ progenitor cells are cultured in 2-dimensional (2D) culture. In some embodiments, the population of CD34⁺ progenitor cells are cultured in 3-dimensional (3D) culture.

In some embodiments, the contacting of the population of CD34⁺ progenitor cells with the fourth culture medium produces a population of T cell progenitor cells.

Provided herein is a population of T cell progenitor cells produced by one or more of the methods provided herein.

In some embodiments, the population of T cell progenitor cells are further dissociated with a mixture of proteolytic and collagenolytic enzymes (e.g., Accutase™). In some embodiments, the population of T cell progenitor cells are dissociated using trypsin-EDTA.

In some embodiments, the population of T cell progenitor cells are cultured in 2D culture on an extracellular matrix (ECM) composition. In other embodiments, the population of T cell progenitor cells are cultured in 3D culture in the presence of an ECM composition. In some embodiments, the ECM composition comprises fibronectin, retronectin, laminin, vitronectin, human Fc-delta-like protein 4, solubilized basement membrane preparation extracted from the Engelbreth-Holm-Swarm (EHS) mouse sarcoma, methylcellulose, collagen, elastin, hyaluronic acid, or any combination thereof.

### Stage 2a to Stage 2b

Further provided herein are methods of producing a population of double positive (CD4⁺ CD8⁺) T cells from a population of T cell progenitor cells. In some embodiments, the methods comprise contacting the population of T cell progenitor cells with a fifth cell culture medium comprising: proteins from mammalian plasma. In some embodiments, the mammalian plasma is human plasma. In some embodiments, the fifth cell culture medium comprises albumin, insulin transferrin, or an antioxidant. In some embodiments, the fifth cell culture medium comprises albumin, insulin transferrin, and an antioxidant. In some embodiments, the fifth cell culture medium comprises one or more growth factors. In some embodiments, the growth factor is insulin-like growth factor. In some embodiments, the fifth cell culture medium comprises a transforming growth factor β receptor family ligand. In some embodiments, the fifth cell culture medium comprises a serine-threonine kinase receptor ligand. In some embodiments, the fifth cell culture medium comprises a glycogen synthase kinase 3 inhibitor. In some embodiments, the fifth cell culture medium does not comprise a glycogen synthase kinase 3 inhibitor. In some embodiments, the fifth cell culture medium comprises an activin receptor ligand. In some embodiments, the fifth cell culture medium does not comprise an activin receptor ligand. In some embodiments, the activin receptor ligand is a ligand of an ALK1 receptor, an ALK2 receptor, an ALK3 receptor, an ALK4 receptor, an ALK5 receptor, an ALK6 receptor, a ALK7 receptor, or any combination thereof. In some embodiments, the fifth cell culture medium comprises an anaplastic lymphoma kinase 5 (ALK5) inhibitor. In some embodiments, the fifth cell culture medium does not comprise an anaplastic lymphoma kinase 5 (ALK5) inhibitor. In some embodiments, the fifth cell culture medium comprises an anaplastic lymphoma kinase 6 (ALK6) inhibitor. In some embodiments, the fifth cell culture medium does not comprise an anaplastic lymphoma kinase 6 (ALK6) inhibitor. In some embodiments, the fifth cell culture medium comprises an anaplastic lymphoma kinase 7 (ALK7) inhibitor. In some embodiments, the fifth cell culture medium does not comprise an anaplastic lymphoma kinase 7 (ALK7) inhibitor. In some embodiments, the fifth cell culture medium comprises a Rho kinase (ROCK) inhibitor. In some embodiments, the fifth cell culture medium does not comprise a ROCK inhibitor. In some embodiments, the Rho kinase (ROCK) inhibitor is selective for ROCK1. In some embodiments, the ROCK inhibitor comprises thiazovivin, Y-27632, Y-30141, Y-33075, Y-39983, or any combination thereof. In some embodiments, the fifth cell culture medium does not comprise thyroid peroxidase. In some embodiments, the fifth cell culture medium comprises thyroid peroxidase. In some embodiments, the fifth cell culture medium comprises a CD135 receptor ligand. In some embodiments, the fifth cell culture medium does not comprise a CD135 receptor ligand. In some embodiment, the CD135 receptor ligand is FMS-like tyrosine kinase 3 ligand (FLT3L, NCBI Gene ID: 2323), an FLT3L fusion protein. In some embodiments, the fifth cell culture medium comprises a CD135 receptor inhibitor. In some embodiments, the fifth cell culture medium does not comprise a CD135 receptor inhibitor. In some embodiments, the CD135 receptor inhibitor comprises gilteritinib, lestaurtinib, midostaurin, sorafenib, or sunitinib.

In some embodiments, the fifth cell culture medium comprises one or more organosulfur compound. Organosulfur compounds are organic compounds that contain sulfur. In some embodiments, the organosulfur compound is allicin, diphenyl disulfide, dibenzothiophene, dimethyl sulfoxide (DMSO), lipoic acid, methionine, penicillin, sulfanilamide. In some embodiments, the fifth cell culture medium comprises dihydrolevoglucosenone. In some embodiments, the fifth cell culture medium comprises an amino acid. Exemplary amino acids include but are not limited to alanine, arginine, cysteine, glutamine, glutamate, histidine, leucine, lysine, methionine, phenylalanine, serine, tryptophan, or tyrosine.

In some embodiments, the fifth cell culture medium comprises one or more cytokine. Exemplary cytokines include but are not limited to: granulocyte-macrophage colony-stimulating factor (GMCSF), IL-2, IL-3, IL-4, IL-6, IL-7, IL-11, IL-15, IL-21, and tumor necrosis factor alpha (TNFα). In some embodiments, the fifth cell culture medium comprises a thrombopoietin receptor ligand. In some embodiments, the fifth cell culture medium does not comprise thrombopoietin (TPO). In some embodiments, the fifth cell culture medium comprises retinoic acid. In some embodiments, the fifth cell culture medium does not comprise retinoic acid.

In some embodiments, the population of T cell progenitor cells are contacted with the fifth cell culture medium for at least about 12 hours, 36 hours, 48 hours, 60 hours, 72 hours, 96 hours, 108 hours, 120 hours, 132 hours, 144 hours, 156 hours, or 168 hours. In some embodiments, the population of T cell progenitor cells are contacted with the fifth cell culture medium for at least about 1 day, 7 days, 14 days, 21 days, 28 days, up to 35 days.

In some embodiments, the population of T cell progenitor cells are cultured in conditions that permit circular rotation. In some embodiments, the circular rotation is at least about 90 rpm, 95 rpm, 100 rpm, 105 rpm, 110 rpm, 115 rpm, 120 rpm, 125 rpm, up to 150 rpm. In some embodiments, the population of T cell progenitor cells are cultured in laminar flow conditions. In some embodiments, the laminar flow rate is at least about 100 microliters per minute (μL/min), about 125 μL/min, about 150 μL/min, about 175 μL/min, about 200 μL/min, about 225 μL/min, about 250 L/min, about 275 μL/min, about 300 μL/min, about 425 μL/min, about 450 μL/min, about 475 μL/min, about 500 μL/min, about 525 μL/min, about 550 μL/min, about 575 μL/min, about 600 L/min, about 625 μL/min, about 650 μL/min, about 675 μL/min, about 700 μL/min, about 725 μL/min, about 750 μL/min, about 775 μL/min, up to 800 μL/min. In some embodiments, the population of T cell progenitor cells are cultured in static conditions. In some embodiments, the culturing is performed for at least about 12 hours, 36 hours, 48 hours, 60 hours, 72 hours, 96 hours, 108 hours, 120 hours, 132 hours, 144 hours, 156 hours, or 168 hours. In some embodiments, the population of T cell progenitor cells are cultured for at least about 1 day, 7 days, 14 days, 21 days, 28 days, up to 35 days.

In some embodiments, the population of T cell progenitor cells are cultured in 2-dimensional (2D) culture on an ECM composition. In other embodiments, the population of T cell progenitor cells are cultured in 3-dimensional (3D) culture in the presence of an ECM composition. In some embodiments, the ECM composition comprises fibronectin, retronectin, laminin, vitronectin, human Fc-delta-like protein 4, solubilized basement membrane preparation extracted from the Engelbreth-Holm-Swarm (EHS) mouse sarcoma, methylcellulose, collagen, elastin, hyaluronic acid, or any combination thereof.

In some embodiments, the contacting of the population of T cell progenitor cells with the fifth cell culture medium produces a population of double positive (CD4⁺ CD8⁺) T cells.

Provided herein are double positive (CD4⁺ CD8⁻) T cells produced by the methods provided herein.

In some embodiments, the double positive (CD4⁻ CD8⁺) T cells are further dissociated with a mixture with proteolytic and collagenolytic enzyme (e.g., Accutase™). In some embodiments, the dissociated double positive (CD4⁺ CD8⁺) T cells are replated into a cell culture vessel or onto an ECM composition. In other embodiments, the dissociated double positive (CD4⁺ CD8⁺) T cells are cultured in 3D in the presence of an ECM composition. In some embodiments, the ECM composition comprises fibronectin, retronectin, laminin, vitronectin, human Fc-delta-like protein 4, solubilized basement membrane preparation extracted from the Engelbreth-Holm-Swarm (EHS) mouse sarcoma, methylcellulose, collagen, elastin, hyaluronic acid, or any combination thereof.

Stage 2 to Stage 3-Stimulation of DP (CD4⁺ CD8⁺) T Cells to Produce SP CD4⁺ T Cells.

Provided here are methods of producing a population of single positive CD4⁺ T cells from a population of double positive (CD4⁺ CD8⁺) T cells. Further provided herein are methods of stimulating, activating, modulating, or binding a CD4 co-receptor to produce a single positive CD4⁺ T cell. Further provided herein are methods of stimulating, activating, modulating, or binding a T cell receptor (TCR). In some embodiments, the methods comprise: contacting the population of double positive (CD4⁺ CD8⁺) T cells with a composition comprising: a plurality of binding agents. In some embodiments, the plurality of binding agents comprise: a CD4 receptor binding agent; and a TCR binding agent. In some embodiments, the contacting produces a population of single positive CD4⁺ T cells. In some embodiments, contacting the population of double positive (CD4⁺ CD8⁺) T cells with the composition is for a period of time of at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 40 days, 45 days, up to 50 days.

In some embodiments, the method further comprises contacting the population of single positive (CD4⁺ CD8⁻) T cells with a second composition comprising a matrix and further comprising: (i) a CD3 binding agent that is bound to the matrix; (ii) a CD3 binding agent that is bound to the matrix and a CD28 binding agent that is bound to the matrix; or (iii) a CD3 binding agent that is bound to the matrix; a CD28 binding agent that is bound to the matrix; and a CD2 binding agent that is bound to the matrix. In some embodiments, the single positive CD4+ T cells have been isolated or purified before contacting them with a second composition comprising a matrix and CD3/CD28/CD2 binding agent(s).

In some embodiments, the composition further comprises a matrix. In some embodiments, the CD4 receptor binding agent and a TCR binding agent are bound to the matrix. In some embodiments, the CD4 receptor binding agent is tethered to the TCR binding agent. In some embodiments, the CD4 receptor binding agent is fused to the TCR binding agent in the form of a fusion protein. In some embodiments, the CD4 receptor binding agent binds to a CD4 receptor on the surface of a double positive (CD4⁺ CD8⁺) T cell. In some embodiments, the CD4 receptor binding agent activates the CD4 receptor. In some embodiments, the TCR binding agent binds to a TCR alpha beta chain (TCRαβ) on the surface of a double positive (CD4⁺ CD8⁺) T cell. In some embodiments, the CD4 receptor binding agent binds to a CD4 receptor on the surface of a double positive (CD4⁺ CD8⁺) T cell. In some embodiments, the CD4 receptor binding agent induces a conformational change in the CD4 receptor. In some embodiments, the TCR binding agent binds to a TCR alpha beta chain (TCRαβ) on the surface of a double positive (CD4⁺ CD8⁺) T cell. In some embodiments, the TCR binding agent binds to a TCR gamma chain on the surface of a double positive (CD4⁺ CD8⁻) T cell. In some embodiments, the TCR binding agent binds to a TCR delta chain on the surface of a double positive (CD4⁺ CD8⁺) T cell. In some embodiments, the TCR binding agent binds to a TCR gamma chain and a TCR delta chain on the surface of a double positive (CD4⁺ CD8⁺) T cell. In some embodiments, the TCR binding agent induces a conformational change in the TCR. In some embodiments, the co-engagement of the CD4 receptor and the TCR via the CD4 receptor binding agent and the TCR receptor binding agent, increases the level of expression of CD4 on the surface of a T cell provided herein or a population thereof relative to a comparable cell or population thereof that has not been by contacted by the CD4 receptor binding agent and the TCR receptor binding agent. Various types of binding agents and matrices that can be used are discussed further below.

In some embodiments, the double positive (CD4⁺ CD8⁺) T cells or the population of single positive CD4⁺ T cells are cultured under static conditions. In some embodiments, the population of double positive (CD4⁺ CD8⁺) T cells or the population of single positive CD4⁺ T cells are cultured on an extracellular matrix (ECM) composition. In some embodiments, the ECM composition modulates Notch signaling in a cell relative to a comparable cell that is not cultured on the ECM composition. In some embodiments, the ECM composition reduces the level of notch in a cell relative to a comparable cell that is not cultured on the ECM composition. In some embodiments, the ECM composition comprises fibronectin, retronectin, laminin, vitronectin, human Fc-delta-like protein 4, solubilized basement membrane preparation extracted from the Engelbreth-Holm-Swarm (EHS) mouse sarcoma, methylcellulose, collagen, elastin, hyaluronic acid, or any combination thereof.

In some embodiments, the double positive (CD4⁺ CD8⁺) T cells or the population of single positive CD4⁺ T cells are cultured on a layer of feeder cells. In some embodiments, the feeder cells are mouse embryonic fibroblasts (MEFs). In some embodiments, the MEFs have been inactivated by gamma irradiation.

In some embodiments, the double positive (CD4⁺ CD8⁺) T cells are further dissociated with a mixture with proteolytic and collagenolytic enzyme (e.g., Accutase™). In some embodiments, the dissociated double positive (CD4⁺ CD8⁺) T cells are replated into a cell culture vessel or onto an ECM composition.

In some embodiments, the method further comprises contacting the population of double positive (CD4⁺ CD8⁺) T cells with one or more cytokines. In some embodiments, the one or more cytokines are selected from the group consisting of: interleukin 7 (IL-7), interleukin 21 (IL-21), interleukin 2 (IL-2), and interleukin 15 (IL-15). In some embodiments, the method further comprises contacting the population of double positive (CD4⁺ CD8⁺) T cells with IL-7 and IL-2. In some embodiments, the method further comprises contacting the population of double positive (CD4⁺ CD8⁺) T cells with a cross-linking agent. In some embodiments, the crosslinking agent is phytohemagglutinin (PHA). In some embodiments, the method further comprises contacting the population of double positive (CD4⁺ CD8⁺) T cells with a CD3 binding agent. In some embodiments, the CD3 binding agent is an anti-CD3 antibody or functional fragment thereof. In some embodiments, the CD3 binding agent binds to the CD3 epsilon (CD38) chain of CD3.

Provided herein is a population of single positive CD4⁺ T cells produced by the methods provided herein.

Further provided herein is a mixed population of T cells. In some embodiments, the mixed population of T cells comprise: a population of single positive CD4⁺ T cells; and a population of double positive (CD4⁺ CD8⁺) T cells. In some embodiments, the population of single positive CD4⁺ T cells is at least about 10% of the total number of cells within the mixed population of T cells. In some embodiments, the population of single positive CD4⁺ T cells is at least about 15% or more, 20% or more, 30% or more, 35% or more, 40% or more, 45% or more, 50% or more, 55% or more, 60% or more, 65% or more, 70% or more, 75% or more, 80% or more, 85% or more, 90% or more, 95% or more, up to 99.99% of the total number of cells within the mixed population of T cells. In some embodiments, the population of single positive CD4″ T cells is at least about 40% of the total number of cells within the mixed population of T cells. In some embodiments, the single positive CD4⁺ T cells are in vitro-differentiated from a CD34⁺ progenitor cell. In some embodiments, the single positive CD4⁺ T cells are produced from a population of double positive (CD4⁺ CD8⁺) T cells by the methods provided herein.

In some embodiments, the single positive CD4⁺ T cells do not comprise a CD8 receptor. In some embodiments, the single positive CD4⁺ T cells comprise a CD4 receptor. CD4 acts as a coreceptor with the T-cell receptor on the surface of T lymphocytes to recognize antigens displayed by an antigen presenting cell in the context of class II MHC molecules. The protein functions to initiate or augment the early phase of T-cell activation. Once activated, CD4⁺ T cells divide rapidly and secrete cytokines that regulate or assist the immune response. Multiple alternatively spliced transcript variants encoding different isoforms of CD4 have been identified.

In some embodiments, the single positive CD4⁺ T cells comprise one or more T cell marker. In some embodiments, the one or more T cell marker is selected from Table 1. Methods of determining the presence or absence of a T cell marker include but are not limited to: polymerase chain reaction, Western blotting, flow cytometry, immunoassays, immunochemistry, microscopy, and sequencing.

TABLE 1
Exemplary CD4 T Cell Markers.

| | Human Gene and Protein NCBI |
|---|---|
| Marker | Accession Nos. |
| zinc finger and BTB | Gene ID: 51043 (SEQ ID NO: 8) |
| domain containing 7B | Protein: NP_001243384.1 (SEQ ID |
| (ZBTB7B) | NO: 9) |
| GATA binding protein 3 | Gene ID: 2625 (SEQ ID NO: 10) |
| (GATA3) | Protein: NP_001002295.1 (SEQ ID |
| | NO: 11) |
| RAR related orphan | Gene ID: 6097 (SEQ ID NO: 12) |
| receptor C (RORC) | Protein: NP_005051.2 (SEQ ID |
| | NO: 13) |
| T-box transcription factor 21 | Gene ID: 30009 (SEQ ID NO: 14) |
| (TBX21) | NP_037483.1 (SEQ ID NO: 15) |
| T-cell surface glycoprotein | Gene ID: 920 (SEQ ID NO: 16) |
| CD4 (CD4) | Protein and Isoforms thereof: |
| | NP_000607.1 (SEQ ID NO: 17) |
| | NP_001181943.1 (SEQ ID NO: 18) |
| | NP_001181944.1 (SEQ ID NO: 19) |
| | NP_001181945.1 (SEQ ID NO: 20) |
| | NP_001181946.1 (SEQ ID NO: 21) |
| | NP_001369636.1 (SEQ ID NO: 22) |
| | NP_001369643.1 (SEQ ID NO: 23) |
| T-cell surface glycoprotein | Gene ID: 919 (SEQ ID NO: 24) |
| CD3 or CD247 | Protein: NP_932170.1 (SEQ ID |
| | NO: 25) |
| IL-2 | Gene ID: 3558 (SEQ ID NO: 26) |
| | Protein: NP_000577.2 (SEQ ID |
| | NO: 27) |
| IL-4 | Gene ID: 3565 (SEQ ID NO: 28) |
| | Protein and Isoforms thereof: |
| | NP_000580.1 (SEQ ID NO: 29) |
| | NP_758858.1 (SEQ ID NO: 30) |
| | NP_001341919.1 (SEQ ID NO: 31) |
| IL-13 | Gene ID: 3596 (SEQ ID NO: 32) |
| | Protein: NP_002179.2 (SEQ ID |
| | NO: 33) |
| IL-17A | Gene ID: 3605 (SEQ ID NO: 34) |
| | Protein: NP_002181.1 (SEQ ID |
| | NO: 35) |
| IL-10 | Gene ID: 3586 (SEQ ID NO: 36) |
| | Proteins: |
| | NP_000563.1 (SEQ ID NO: 37) |
| | NP_001369553.1 (SEQ ID NO: 38) |
| IL-21 | Gene ID: 59067 (SEQ ID NO: 39) |
| | Protein: NP_001193935.1 (SEQ ID |
| | NO: 40) |
| IL-9 | Gene ID: 3578 (SEQ ID NO: 41) |
| | Protein: NP_000581.1 (SEQ ID |
| | NO: 42) |
| IL-22 | Gene ID: 50616 (SEQ ID NO: 43) |
| | Protein: NP_065386.1 (SEQ ID |
| | NO: 44) |
| BCL-6 | Gene ID: 54880 (SEQ ID NO: 45 |
| | Protein and isoforms thereof: |
| | NP_060215.4 (SEQ ID NO: 46) |
| | NP_001116856.1 (SEQ ID NO: 47) |
| | NP_001116857.1 (SEQ ID NO: 48) |
| Interferon gamma | Gene ID: 3458 (SEQ ID NO: 49) |
| (IFNG or IFNγ) | Protein: NP_000610.2 (SEQ ID |
| | NO: 50) |
| IKAROS family zinc finger 2 | Gene ID: 22807 (SEQ ID NO: 51 |
| (IKZF2 or HELIOS) | Protein: NP_001374149.1 (SEQ ID |
| | NO: 52) |
| Forkhead box P3 (FOXP3) | Gene ID: 50943 (SEQ ID NO: 53) |
| | Protein: NP_054728.2 (SEQ ID |
| | NO: 54) |

Further provided herein are a population of CD4⁺ T cells that produce cytokines. In some embodiments, the population of CD4⁺ T cells provided herein comprise T helper 1 (Th1), Th2 effector T cells, T helper type 9 (Th9) cells, Follicular B helper T cells, T helper 17 (Th17) cells, or any combination thereof. Further provided herein are methods for generating a subpopulation of CD4⁺ T cells. In some embodiments, the methods comprise: contacting the population of double positive (CD4⁺ CD8⁻) T cells or the population of single positive CD4⁺ T cells with a cytokine or a transcription factor provided herein, thereby producing a subpopulation of CD4⁺ T cells. CD4⁺ T cells provided herein can differentiate into T helper and T regulatory cells via polarizing cytokine signals added to the cell culture medium. In some embodiments, the subpopulation of CD4⁺ T cells comprise a population of Th1 cells, a population of Th2 cell, a population of Th9 cell, a population of Th17 cells, a population of follicular B helper T cells, a population of T regulatory cells (Tregs), or any combination thereof.

In some embodiments, the methods further comprise contacting the population of double positive (CD4⁺ CD8⁺) T cells or the population of single positive CD4⁺ T cells with one or more cytokine selected from the group consisting of: IL-7, IL-21, IL-2, IL-1, IL-4, IL-9, IL-10, IL-13, IL-15, IL-24, IL-25, IL-33, and IFNγ, thereby producing a CD4⁺ T cell subpopulation. In some embodiments, the methods further comprise contacting the population of double positive (CD4+CD8⁺) T cells or the population of single positive CD4⁺ T cells with a transcription factor. In some embodiments, the transcription factor is inducible T-cell co-stimulator, CD278 or ICOS. In some embodiments, the methods further comprise contacting the population of double positive (CD4+CD8⁺) T cells or the population of single positive CD4⁺ T cells with TGF-β. In some embodiments, the methods further comprise contacting the population of double positive (CD4⁺ CD8⁺) T cells or the population of single positive CD4⁺ T cells with one or more factor selected from the group consisting of: STAT6, IRF4, GATA3, PU.1, BATF, NF-κB, NFAT1, STAT5, AP-1, or any combination thereof.

In some embodiments, methods further comprise contacting the population of double positive (CD4⁺ CD8⁺) T cells or the population of single positive CD4⁺ T cells with one or more factor selected from activin A, Jagged2, programmed cell death ligand (PD-L2), cyclooxygenase (COX)-2, 1,25-dihydroxyvitamin D3, calcitonin gene-related peptide (CGRP), tumor necrosis factor receptor superfamily member 4 (TNFRSF4 or OX40), thymic stromal lymphopoietin (TSLP), or any combination thereof.

In some embodiments, the single positive CD4⁺ T cells provided herein are further contacted with an antigen or a binding agent that binds to an antigen. In some embodiments, the single positive CD4⁺ T cells provided herein are further contacted with a plurality of antigens. In some embodiments, the single positive CD4⁺ T cells provided herein are further contacted with a plurality of binding agents. In some embodiments, the antigen is a tumor antigen or a microbial antigen. In some embodiments, the single positive CD4⁺ T cells provided herein are further contacted with a B-cell maturation antigen (BCMA) binding agent, a prostate-specific membrane antigen (PSMA) binding agent, a CD3 binding agent, or a combination thereof.

### (2) Single Positive CD8⁺ T Cells and Methods of Producing the Same

Provided herein are methods for producing a population of single positive CD8⁺ T cells from a population of double positive (CD4⁺ CD8⁺) T cells. Further provided herein are methods of stimulating, activating, modulating, or binding a CD8 co-receptor to produce a single positive CD8⁺ T cell. Further provided herein are methods of stimulating, activating, modulating, or binding a T cell receptor (TCR). In some embodiments, the methods comprise: contacting a double positive (CD4⁺ CD8⁺) T cell with a composition comprising: a plurality of binding agents. In some embodiments, the plurality of binding agents comprise: a CD8 receptor binding agent; and a TCR binding agent. In some embodiments, the contacting produces a single positive CD8⁺ T cell. In some embodiments, the composition further comprises a matrix. In some embodiments, the CD8 receptor binding agent and a TCR binding agent are bound to the matrix. In some embodiments, the CD8 receptor binding agent is tethered to the TCR binding agent. In some embodiments, the CD8 receptor binding agent is fused to the TCR binding agent in the form of a fusion protein. In some embodiments, the CD8 receptor binding agent binds to a CD8 receptor on the surface of a double positive (CD4⁺ CD8⁺) T cell, thereby activating the CD8 receptor. In some embodiments, the TCR binding agent binds to a TCR alpha beta chain (TCRαβ) on the surface of a double positive (CD4⁺ CD8⁺) T cell, thereby activating the TCR. In some embodiments, the CD8 receptor binding agent binds to a CD8 receptor on the surface of a double positive (CD4⁺ CD8⁺) T cell. In some embodiments, the CD8 receptor binding agent induces a conformational change in the CD8 receptor. In some embodiments, the TCR binding agent binds to a TCR alpha beta chain (TCRαβ) on the surface of a double positive (CD4⁺ CD8⁺) T cell. In some embodiments, the TCR binding agent induces a conformation change in the TCR. In some embodiments, the co-engagement of the CD8 receptor and the TCR via the CD8 receptor binding agent and the TCR receptor binding agent, increases the level of expression of CD8 on the surface of a T cell provided herein or a population thereof relative to a comparable cell or population thereof that has not been by contacted by the CD8 receptor binding agent and the TCR receptor binding agent. Various types of binding agents and matrices that can be used are discussed further below.

In some embodiments, the double positive (CD4⁺ CD8⁺) T cells or the population of single positive CD4⁺ T cells are cultured under static conditions. In some embodiments, the ECM composition increases the level of notch in a cell relative to a comparable cell that is not contacted with the ECM composition. In some embodiments, the population of double positive (CD4⁺ CD8⁺) T cells or the population of single positive CD4⁺ T cells are cultured on an extracellular matrix (ECM) composition. In some embodiments, the ECM composition comprises fibronectin, retronectin, laminin, vitronectin, human Fc-delta-like protein 4, solubilized basement membrane preparation extracted from the Engelbreth-Holm-Swarm (EHS) mouse sarcoma, methylcellulose, collagen, elastin, hyaluronic acid, or any combination thereof.

In some embodiments, the contacting the population of double positive (CD4⁺ CD8⁺) T cells with the composition is for a period of time of at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 40 days, 45 days, up to 50 days.

In some embodiments, the method further comprises contacting the population of double positive (CD4⁺ CD8⁺) T cells with one or more cytokines. In some embodiments, the one or more cytokines are selected from the group consisting of: IL-7, IL-21, IL-2, and IL-15. In some embodiments, the method further comprises contacting the population of double positive (CD4+CD8⁺) T cells with IL-7 and IL-2.

In some embodiments, the method further comprises contacting the population of double positive (CD4⁻ CD8⁺) T cells with a second composition comprising a matrix and further comprising: (i) a CD3 binding agent that is bound to the matrix; (ii) a CD3 binding agent that is bound to the matrix and a CD28 binding agent that is bound to the matrix; or (iii) a CD3 binding agent that is bound to the matrix; a CD28 binding agent that is bound to the matrix; and a CD2 binding agent that is bound to the matrix.

Provided herein is a population of single positive CD8⁺ T cells produced by the methods provided herein.

Further provided herein is a mixed population of T cells. In some embodiments, the mixed population of T cells comprise: a population of single positive CD8⁺ T cells; and a population of double positive (CD4⁺ CD8⁺) T cells. In some embodiments, the population of in vitro-differentiated single positive CD8⁺ T cells is at least about 10% of the total number of cells within the mixed population of in vitro-differentiated T cells. In some embodiments, the population of in vitro-differentiated CD8 single positive T cells is at least about 20% or more, 30% or more, 40% or more, 50% or more, 60% or more, 70% or more, 80% or more, 90% or more, up to 99.99% of the total number of cells within the mixed population of in vitro-differentiated T cells.

In some embodiments, the single positive CD8⁺ T cells comprise a CD8 receptor. In some embodiments, the single positive CD8⁺ T cells comprise one or more T cell marker. In some embodiments, the one or more T cell marker is selected from Table 2. Methods of determining the presence or absence of a T cell marker include but are not limited to: polymerase chain reaction, Western blotting, flow cytometry, immunoassays, immunochemistry, microscopy, and sequencing.

TABLE 2
Exemplary CD8 T Cell Markers.

| | Human Gene and Protein NCBI |
|---|---|
| Marker | Accession Nos. |
| RUNX family transcription | Gene ID: 864 |
| factor 3 (RUNX3) | Protein: NP_004341.1 (SEQ ID NO: |
| | 55) |
| tumor necrosis factor | Gene ID: 7124 |
| alpha (TNFα) | Protein: NP_000585.2 (SEQ ID NO: |
| | 56) |
| granzyme B | Granzyme B: Gene ID: 3002 |
| | Granzyme B Protein: NP_004122.2 |
| | (SEQ ID NO: 57); NP_001332940.1 |
| T-cell surface glycoprotein | Gene ID: 919 |
| CD3 or CD247 | Protein: NP_932170.1 (SEQ ID NO: |
| | 58) |
| perforin 1 | Gene ID: 5551 |
| | Protein and Isoforms thereof: |
| | NP_001076585.1 (SEQ ID NO: 59); |
| | NP_005032.2 |
| T-cell surface glycoprotein | Gene ID: 925 |
| CD8 alpha chain (CD8A) | Protein and Isoforms thereof: |
| | NP_001759.3 (SEQ ID NO: 60) |
| | NP_741969.1 |
| | NP_001139345.1 |
| | NP_001369627.1 |
| T-cell surface glycoprotein | Gene ID: 926 |
| CD8 beta chain (CD8B) | Protein and Isoforms thereof: |
| | NP_001171571.1 (SEQ ID NO: 61) |
| | NP_004922.1 |
| | NP_742099.1 |
| | NP_742100.1 |
| | NP_757362.1 |
| Interferon gamma | Gene ID: 3458 |
| (IFNG or IFNγ) | Protein: NP_000610.2 |
| | (SEQ ID NO: 50) |

### (3) Cell Sources

Provided herein are compositions comprising single positive CD4⁺ T cells or single positive CD8⁺ T cells, wherein the single positive CD4⁺ T cells or single positive CD8⁺ T cells are derived from a population of stem cells. In some embodiments, the single positive CD4⁺ T cells or single positive CD8⁺ T cells are in vitro-differentiated from a population of stem cells. In some embodiments, the population of stem cells comprise an embryonic stem (ES) cell. Embryonic stem cells are considered to be undifferentiated cells, which are not committed to a specific differentiation lineage. Such cells display morphological characteristics that distinguish them from differentiated cells of embryo or adult origin. Undifferentiated ES cells typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines do not stain for SSEA-1, but differentiated cells stained strongly for SSEA-I.

In some embodiments, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells. In some embodiments, the population of stem cells comprise an induced pluripotent stem cell (iPSC). Reprogramming is a process that generates a cell that re-expresses a stem cell phenotype or a less differentiated phenotype than the cell from which it is derived. For example, a multipotent cell can be dedifferentiated to a pluripotent cell. That is, dedifferentiation shifts a cell backward along the differentiation spectrum of totipotent cells to fully differentiated cells. Typically, reversal of the differentiation phenotype of a cell includes artificial manipulation of the cell, for example, by expressing stem cell-specific mRNA and/or proteins. Reprogramming is not typically observed under native conditions in vivo or in vitro. Methods of reprogramming differentiated cells to iPS cells involve forced expression of Oct 3/4, Sox2, Klf4, and c-Myc in the cells, although numerous variations are known in the art. iPSCs can be cultured, expanded, and passaged according to the methods described herein or other conditions favorable to cell viability and maintenance of the undifferentiated, pluripotent phenotype.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-111-tubulin; α-smooth muscle actin (ot-SMA); fibroblast growth factor 4 (Fgf4), Cripto, Daxl; zinc finger protein 296 (Zfp296); N-acetyltransferase-1 (Natl); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthll7; Sall4; undifferentiated embryonic cell transcription factor (Utfl); Rexl; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tell); DPPA3/Stella; DPPA4; other general markers for pluripotency, etc. Other markers can include Dnmt3L; Sox15; Stat3; Grb2; β-catenin, and Bmil. Such cells can also be characterized by the down-regulation of markers characteristic of the somatic cell from which the induced pluripotent stem cell is derived. In some embodiments, the population of induced pluripotent stem cells is derived from a population of human skin cells, human umbilical cord cells, human blood cells or a human tissue.

Exemplary stem cells include adult stem cells, neural stem cells, liver stem cells, muscle stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. One of skill in the art can select a stem cell or other somatic cell for the purpose of generating stem-cell derived single positive T cells based on, for example, the ease of obtaining a sample from a subject. Stem cells that can be relatively non-invasively obtained from a subject include hematopoietic stem cells, umbilical cord stem cells, and skin stem cells.

In some embodiments, the pluripotent stem cell is an adult stem cell. Adult stem cells are multipotent stem cells derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include hematopoietic stem cells, mesenchymal stem cells, neural stem cells, neural crest stem cells, and pancreatic stem cells. In some embodiments, the adult stem cells are extracted from or isolated from a healthy subject. In some embodiments, the adult stem cells are extracted from or isolated from a subject that has, is suspected of having, or is at risk of developing a disease.

The cells provided herein can be from any human, non-human, or mammalian source. In some embodiments, the population of cells that can be used in the methods provided herein include, for example, human pluripotent stem cells (hPSC), primate pluripotent stem cells, mouse pluripotent stem cells, rat pluripotent stem cells, pig pluripotent stem cells, canine pluripotent stem cells, feline pluripotent stem cells, equine pluripotent stem cells. In some embodiments, the population of cells are derived from umbilical cord blood, skin, urine, blood, or a tissue biopsy. In some embodiments, the cells are extracted from or isolated from a healthy subject. In some embodiments, the cells are extracted from or isolated from a subject that has, is suspected of having, or is at risk of developing a disease.

Provided herein are compositions comprising in vitro-differentiated T cells, wherein the in vitro-differentiated T cells are derived from a population of CD34⁺ progenitor cells, a population of T cell progenitor cells, or a population of double positive (CD4⁺ CD8⁺) T cells. Further provided herein are compositions comprising single positive CD4⁺ T cells, wherein the single positive CD4⁺ T cells are derived from a population of CD34⁺ progenitor cells, a population of T cell progenitor cells, or a population of double positive (CD4⁺ CD8⁺) T cells. Further provided herein are compositions comprising single positive CD8⁺ T cells, wherein the single positive CD8⁺ T cells are derived from a population of CD34⁺ progenitor cells, a population of T cell progenitor cells, or a population of double positive (CD4⁺ CD8⁺) T cells. In some embodiments, the single positive CD4⁺ T cells or the single positive CD8⁺ T cells are derived from a progenitor cell that comprises a marker listed in Table 3.

Cells used in the compositions and methods provided herein can be obtained from a number of non-limiting sources, including, for example, peripheral blood mononuclear cells, bone marrow, lymph node tissue, cord blood, thymus tissue, tissue from a site of infection, ascites, pleural effusion, spleen tissue, and tumors. A cell provided herein can be derived from a healthy donor, from a patient diagnosed with a disease or a disorder, for example, a cell from a subject that has a cancer or an autoimmune disease, or from a patient diagnosed with an infection. In another embodiment, the cell can be part of a mixed population of cells which present different phenotypic characteristics. A cell can also be obtained from a cell therapy bank. A desirable cell population can also be selected by known methods, which include but are not limited to cell magnetic separation, flow cytometric selection, or antibiotic selection.

In some embodiments, the in vitro-differentiated T cells provided herein are derived from human umbilical cord blood cells (HUCBCs). HUCBCs contain mesenchymal and hematopoietic progenitor cells, and endothelial cell precursors that can be expanded in tissue culture.

Provided herein are compositions comprising single positive CD4⁺ T cells or the single positive CD8⁺ T cells, wherein the single positive CD4⁺ T cells or the single positive CD8⁺ T cells are derived from a population of genetically modified cells. The population of genetically modified cells can include, but are not limited to stem cells that comprise a genomic disruption in a target gene, stem cells that comprise an exogenous transgene, stem cells that comprise a genomic disruption in a target gene and express an exogenous transgene. In some embodiments, the exogenous transgene is inserted into an endogenous gene locus. In some embodiments, the endogenous gene locus is a TRAC locus, a TRBC locus, a CD3δ locus, a CD3ε locus, a CD247 locus, a B2M locus, a TRGC locus, or a TRDC locus. In some embodiments, the exogenous transgene comprises a gene or a marker listed in Table 3.

Methods of genetically modifying a cell can include but is not limited to: molecular cloning techniques, CRISPR, TALENS, zinc fingers, and introduction of a vector to a cell, for example, plasmids, adenoviral vectors, lentiviral vectors, non-viral vectors, or minicircle vectors.

The cells used in the compositions and methods provided herein can be characterized by several methods for cell morphology, cell marker expression, or cellular function. For example, cellular function can be assessed by a cytotoxicity assay, an antibody titer assay, or an immunoassay for the production of interleukins and molecules associated with an immune response.

Cellular compositions provided herein can be cryopreserved. A cryopreservation can be performed in, for example, about a 5% (w/v) DMSO solution. A cryopreservation can be at a freeze density from about 1.0×10⁷ cells/mL to about 8.0×10⁸ cells/mL. A freeze density can be from about 1×10⁷ cells/mL, 1.5×10⁷ cells/mL, 2×10⁷ cells/mL, 2.5×10⁷ cells/mL, 3×10⁷ cells/mL, 3.5×10⁷ cells/mL, 4×10⁷ cells/mL, 4.5×10⁷ cells/mL, 5×10⁷ cells/mL, 5.5×10⁷ cells/mL, 6×10⁷ cells/mL, 6.5×10⁷ cells/mL, 7×10⁷ cells/mL, 7.5×10⁷ cells/mL, 8×10⁷ cells/mL, 8.5×10⁷ cells/mL, 9×10⁷ cells/mL, 9.5×10⁷ cells/mL, 1×10⁸ cells/mL, 1.5×10⁸ cells/mL, 2×10⁸ cells/mL, 2.5×10⁸ cells/mL, 3×10⁸ cells/mL, 3.5×10⁸ cells/mL, 4×10⁸ cells/mL, 4.5×10⁸ cells/mL, 5×10⁸ cells/mL, 5.5×10⁸ cells/mL, 6×10⁸ cells/mL, 6.5×10⁸ cells/mL, 7×10⁸ cells/mL, 7.5×10⁸ cells/mL, or up to about 8×10⁸ cells/mL.

For example, a population of single positive T cells provided herein (e.g., SP CD4⁺ or SP CD8⁺ T cells) can be harvested, washed, and re-suspended in a buffer. In some cases, a cellular composition is thawed prior to an introducing into a subject in need thereof.

Cellular viability of a population of single positive T cells herein (e.g., SP CD4″ or SP CD8⁺ T cells) produced by the methods provided herein can be determined by flow cytometry and trypan blue exclusion. In some cases, at least about 50% cells can be viable for administration. In other cases from about 50%, 60%, 70%, 80%, 90%, 95%, or up to about 100% cells can be viable.

Provided herein are a population of pluripotent stem cells, a population of mesodermal stem cells, a population of early hematopoietic progenitor cells, a population of CD34⁺ hematopoietic progenitor cells, a population of T cell progenitor cells, a population of DP CD4⁺/CD8⁺ T cells, a population of SP CD4⁺ T cells, and a population of SP CD8⁺ T cells. The cells produced by the methods provided herein can be characterized for the presence or absence of a marker or a set of markers. Methods of characterizing the expression of a cell marker can include but is not limited to, PCR, sequencing, Western blot, or immunostaining. In some embodiments, a cell provided herein expresses a marker in Table 3.

TABLE 3
Exemplary Cell Markers

| Cell type | Exemplary Cell Markers |
|---|---|
| Pluripotent Stem Cell | OCT4, SOX2 and NANOG |
| CD34⁺ progenitor cell | CD34, CD45, CD43, CD62L |
| T cell progenitor cell | CD34, CD7, CD5, CD4, |
| | VISTA (PD-1H), |
| | CD62L (L-selectin), CD127, CD197 |
| Double positive CD4⁺/CD8⁺ | CD4, CD8, CD3, TCRαβ, TCRγδ, |
| T cell | CD44, CD25, CD69, CD71, CD98 |
| Single positive CD4⁺ T cell | CD4, ZBTB7B (ThPOK), CD3, |
| | TBX21, IL-2, IFNγ, |
| | GATA3, IL-4, IL-13, RORC, |
| | IL-17, FOXP3, HELIOS, |
| | IL-10, BCL6, IL-21, IL-9, IL-22 |
| Single positive CD8⁺ T cell | CD8A, CD8B, RUNX3, CD3, |
| | IFNG, TNFA, GZMB |

### (4) Binding Agent Compositions

Provided herein are compositions for use in the production of single positive T cells. In some embodiments, the compositions comprise a CD4 binding agent; a CD8 binding agent, and/or a TCR binding agent. In some embodiments, the binding agent is a small molecule. In some embodiments, the binding agent is a nucleic acid. In some embodiments, the binding agent is a protein. In some embodiments, the binding agent is an aptamer.

In some embodiments, the binding agent is an antibody or a functional fragment thereof. In some embodiments, the antibody is a monoclonal antibody. Monoclonal antibodies or mAbs include intact molecules, as well as antibody fragments (such as, Fab and F(ab′)2 fragments) that are capable of specifically binding to an epitope of a protein or antigen. In some embodiments, the composition comprises nucleic acids encoding for polyclonal antibody.

In some embodiments, the antibody is a murine antibody, a humanized antibody, or a fully human antibody, or a single domain heavy chain antibody derived from camelids, sharks, eels, or other species that produce such single-domain antibodies. In some embodiments, the antibody is an immunoglobulin (Ig) molecule. Immunoglobulin (Ig) molecules and immunologically active portions of immunoglobulin molecules (i.e., molecules that contain an antigen binding site that specifically bind an antigen) are comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies. In some embodiments, the immunoglobulin molecule is an IgG, IgE, IgM, IgD, IgA, or an IgY isotype immunoglobulin molecule. In some embodiments, the antibody or immunoglobulin molecules provided herein are a specific subclass of immunoglobulin molecule. In some embodiments, the immunoglobulin molecule is an IgG1, an IgG2, an IgG3, an IgG4, an IgGA1, or an IgGA2 subclass immunoglobulin molecule. In some embodiments, the immunoglobulin molecule is an IgG1 subclass immunoglobulin molecule. In a full-length antibody, each heavy chain is comprised of a heavy chain variable domain (abbreviated herein as HCVR or V_{H}) and a heavy chain constant region. The heavy chain constant region is comprised of three domains: C_{H}1, C_{H}2, and C_{H}3. Each light chain is comprised of a light chain variable domain (abbreviated herein LCVR as V_{L}) and a light chain constant region. The light chain constant region is comprised of one domain, CL. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. This structure is well-known to those skilled in the art. The chains are usually linked to one another via disulfide bonds. Furthermore, in humans, the light chain may comprise a kappa chain or a lambda chain. Complementarity Determining Regions (“CDRs”), i.e., CDR1, CDR2, and CDR3) are the amino acid residues of a heavy or light chain variable domain specific for antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (HI), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (HI), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. The exact boundaries of these CDRs have been defined differently according to different systems. The system described by Kabat (Kabat et al, Sequences of Proteins of Immunological Interest (National Institutes of Health, Bethesda, Md. (1987) and (1991)) not only provides an unambiguous residue numbering system applicable to any variable region of an antibody, but also provides the residue boundaries defining the three CDRs. These CDRs may be referred to as Kabat CDRs. Chothia and coworkers (Chothia & Lesk, J. Mol. Biol, 196:901-917 (1987) and Chothia et al., Nature 342:877-883 (1989)) found that certain sub-portions within Kabat CDRs adopt nearly identical peptide backbone conformations, in spite of great diversity at the level of amino acid sequence. These sub-portions were designated as L1, L2 and L3 or HI, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB). 9:133-139 (1995)) and MacCallum (J Mol Biol 262(5): 732-45 (1996)). Still other CDR boundary definitions may not strictly follow one of the above systems, but will nonetheless overlap with the Kabat CDRs, although they may be shortened or lengthened in light of prediction or assay result that particular residues or groups of residues or even entire CDRs do not significantly impact antigen binding. The alignment of the CDR sequences can be conducted using publicly available software such as BLAST, Align, and the international ImMunoGeneTics information system (IMGT). Those skilled in the art can determine the appropriate parameters for alignment, but the default parameters for BLAST are specifically contemplated.

In some embodiments, the antibody is a recombinant antibody, a chimeric antibody, or a multivalent antibody. In some embodiments, the multivalent antibody is a bispecific antibody, a trispecific antibody, or a multispecific antibody. In some embodiments, the antibody or functional fragment is an antigen-binding fragment (Fab), and Fab2 a F(ab′), a F(ab′)2, an dAb, an Fc, a Fv, a disulfide linked Fv, a scFv, a tandem scFv, a free LC, a half antibody, a single domain antibody (dAb), a diabody, or a nanobody. In some embodiments, nucleic acids provided herein encode for a single variable domain on a heavy chain (also referred to as a nanobody or a V_{H}H). In some embodiments, the nanobody comprises a heavy chain variable (V_{H}) region. In some embodiments, the nanobody comprises one CDR region. In some embodiments, the nanobody comprises CDR1, CDR2, or CDR3. In further embodiments, the heavy chain variable (V_{H}) region comprises three CDR regions. In some embodiments, the antibody, nanobody, or fragment thereof is modified with the addition of a glycosylphosphatidylinositol (GPI) anchor such as that derived from CD55, or with a human Fc domain, or a combination of Fc and GPI.

In some embodiments, the CD4 receptor binding agent comprises tregalizumab, ibalizumab, CAMPATH-9, zanolimumab, OKT4, keliximab, clenoliximab, adnectin (6940_B01), SK3, MT310, RPA-T4, QS4120, B-A1, EDU-2, MT441, a variant, a fusion, or a combination thereof. In some embodiments, the CD4 binding agent binds to an amino acid sequence that is at least 80% identical to SEQ ID NO: 1, SEQ ID NO: 4, a variant, or a functional fragment thereof. In some embodiments, the CD4 binding agent binds to SEQ ID NO: 1, a variant, or a functional fragment thereof. In some embodiments, the CD4 binding agent binds to SEQ ID NO: 4, a variant, or a functional fragment thereof.

In some embodiments, the TCR binding agent comprises anti-TCR-IARC307 (e.g., Clone IP26) or anti-TCR-H57. In some embodiments, the TCR binding agent binds to a TCRαβ chain or a functional fragment thereof. In some embodiments, the TCR binding agent binds to a TCR gamma chain or a functional fragment thereof. In some embodiments, the TCR binding agent binds to a TCR delta chain or a functional fragment thereof. In some embodiments, the TCR binding agent binds to an amino acid sequence that is at least 80% identical to SEQ ID NO: 2, SEQ ID NO: 5, SEQ ID NO: 6, SEQ ID NO: 7, a variant, or a functional fragment thereof. In some embodiments, the TCR binding agent binds to SEQ ID NO: 2 or a functional fragment thereof. In some embodiments, the TCR binding agent binds to SEQ ID NO: 5, SEQ ID NO: 6, SEQ ID NO: 7, a variant, or a functional fragment thereof.

In some embodiments, the CD8 receptor binding agent comprises anti-CD8a-G10-1, MA5-14548, or anti-CD8a-OKT8. In some embodiments, the CD8 receptor binding agent binds to a CD8 receptor or a functional fragment thereof. In some embodiments, the CD8 receptor binding agent binds to a sequence that is at least 80% identical to SEQ ID NO: 3, a variant, or a functional fragment thereof. In some embodiments, the CD8 receptor binding agent binds to SEQ ID NO: 3, a variant, or a functional fragment thereof.

In some embodiments, a binding agent provided herein binds to a protein listed in Table 4.

TABLE 4
Epitope Sequences for Binding Agents.

| Binding Agent | Epitope | Epitope Sequences |
|---|---|---|
| CD4 receptor | CD4 | MNRGVPFRHL LLVLQLALLP AATQGKKVVL GKKGDTVELT |
| binding agent | | CTASQKKSIQ FHWKNSNQIK ILGNQGSFLT KGPSKLNDRA DSRRSLWDQG |
| | | NFPLIIKNL (SEQ ID NO: 1) |
| | | MNRGVPFRHLLLVLQLALLPAATQGKKVVLGKKGDTVELTCTASQKKSIQF |
| | | HWKNSNQIKILGNQGSFLTKGPSKLNDRADSRRSLWDQGNFPLIIKNLKIED |
| | | SDTYICEVEDQKEEVQLLVFGLTANSDTHLLQGQSLTLTLESPPGSSPSVQCR |
| | | SPRGKNIQGGKTLSVSQLELQDSGTWTCTVLQNQKKVEFKIDIVVLAFQKAS |
| | | SIVYKKEGEQVEFSFPLAFTVEKLTGSGELWWQAERASSSKSWITFDLKNKE |
| | | VSVKRVTQDPKLQMGKKLPLHLTLPQALPQYAGSGNLTLALEAKTGKLHQ |
| | | EVNLVVMRATQLQKNLTCEVWGPTSPKLMLSLKLENKEAKVSKREKAVW |
| | | VLNPEAGMWQCLLSDSGQVLLESNIKVLPTWSTPVQPMALIVLGGVAGLLL |
| | | FIGLGIFFCVRCRIIRRRQAERMSQIKRLLSEKKTCQCPIIRFQKTCSPI (SEQ |
| | | ID NO: 4) |
| TCR binding | TCR alpha | GCPALPTGVG GTPFPSLAPP IMLLVDGKQQ MVVVCLVLDV APPGLDSPIW |
| agent | beta chain | FSAGNGSALD AFTYGPSPAT DGTWTNLAHL SLPSEELASW EPLVCHTGPG |
| | | AEGHSRSTQP MHLSGEASTA RTCPQEPLRG TPGGALWLGV LRLLLFKLLL |
| | | FDLLLTCSCL CDPAGPLPSP ATTTRLRALG SHRLHPATET GGREATSSPR |
| | | PQPRDRRWGD TPPGRKPGSP VWGEGSYLSS YPTCPAQAWC SRSRLRAPSS |
| | | SLGAFFAGDL PPPLQAGAA (SEQ ID NO: 2) |
| | | AGSHMGVSQSPRYKVAKRGQDVALRCDPISGHVSLFWYQQALGQGPEFLT |
| | | YFQNEAQLDKSGLPSDRFFAERPEGSVSTLKIQRTQQEDSAVYLCASSLGQA |
| | | YEQYFGPGTRLTVTEDLKNVFPPEVAVFEPSEAEISHTQKATLVCLATGFYP |
| | | DHVELSWWVNGKEVHSGVSTDPQPLKEQPALNDSRYCLSSRLRVSATFWQ |
| | | NPRNHFRCQVQFYGLSENDEWTQDRAKPVTQIVSAEAWGRADCTSGDDDD |
| | | K (SEQ ID NO: 5) |
| | | MNAGVTQTPKFRILKIGQSMTLQCAQDMNHNYMYWYRQDPGMGLKLIYY |
| | | SVGAGITDKGEVPNGYNVSRSTTEDFPLRLELAAPSQTSVYFCASSFATEAFF |
| | | GQGTRLTVVEDLNKVFPPEVAVFEPSEAEISHTQKATLVCLATGFYPDHVEL |
| | | SWWVNGKEVHSGVCTDPQPLKEQPALNDSRYALSSRLRVSATFWQDPRNH |
| | | FRCQVQFYGLSENDEWTQDRAKPVTQIVSAEAWGRAD (SEQ ID NO: 6) |
| | | MSISLLCCAAFPLLWAGPVNAGVTQTPKFRILKIGQSMTLRCTQDMNHNYM |
| | | YWYRQDPGMGLKLIYYSVGAGITDKGEVPNGYNVSRSTTEDFPLRLELAAP |
| | | SQTSVYFCASSYDSPETQYFGPGTRLLVLEDLKNVFPPEVAVFEPSEAEISHT |
| | | QKATLVCLATGFYPDHVELSWWVNGKEVHSGVSTDPQPLKEQPALNDSRY |
| | | CLSSRLRVSATFWQNPRNHFRCQVQFYGLSENDEWTQDRAKPVTQIVSAEA |
| | | WGRADCGFTSESYQQGVLSATILYEILLGKATLYAVLVSALVLMAMVKRK |
| | | DSRG (SEQ ID NO: 7) |
| CD8 receptor | CD8a | MALPVTALLLPLALLLHAARPSQFRVSPLDRTWNLGETVELKCQVLLSNPTS |
| binding agent | | GCSWLFQPRGAAASPTFLLYLSQNKPKAAEGLDTQRFSGKRLGDTFVLTLS |
| | | DFRRENEGYYFCSALSNSIMYFSHFVPVFLPAKPTTTPAPRPPTPAPTIASQPL |
| | | SLRPEACRPAAGGAVHTRGLDFACDIYIWAPLAGTCGVLLLSLVITLYCNHR |
| | | NRRRVCKCPRPVVKSGDKPSLSARYV (SEQ ID NO: 3) |

In some embodiments, an antibody, an antibody fragment, or a nanobody provided herein is originally generated by a non-human animal (e.g., sheep, dog, rabbit, mouse, rat, non-human primate, goat, llama, alpaca, camels, and horse) against a protein or an epitope provided herein and, optionally, humanized as described herein.

Further provided herein are fusion proteins comprising a CD4 receptor binding agent and a TCR binding agent. Further provided herein are fusion proteins comprising a CD8 receptor binding agent and a TCR binding agent. In some embodiments, the fusion protein comprises a linker between the CD4 receptor binding agent and the TCR binding agent. In some embodiments, the fusion protein comprises a linker between the CD8 receptor binding agent and the TCR binding agent. In some embodiments, the CD4 receptor binding agent binds to an amino acid sequence that is at least 80% identical to SEQ ID NO: 1 or a functional fragment thereof. In some embodiments, the CD8 receptor binding agent binds to an amino acid sequence that is at least 80% identical to SEQ ID NO: 3 or a functional fragment thereof. In some embodiments, the TCR binding agent binds to an amino acid sequence that is at least 80% identical to SEQ ID NO: 2 or a functional fragment thereof.

### (5) Matrix Compositions

Provided herein are compositions for use in the production of single positive T cells, comprising a matrix; a CD4 binding agent; and a TCR binding agent. Further provided herein are compositions for use in the production of single positive T cells, comprising a matrix; a CD8 binding agent; and a TCR binding agent. In some embodiments, the surface of the matrix is coated with a plurality of binding agents, for example, antibodies, antibody fragments, or proteins.

In some embodiments, the matrix is a solid support. In some embodiments, the solid support comprises a superparamagnetic colloidal material, a metal, an acrylic, a glass, a plastic, a polystyrene, a thermoplastic polymer, a polytetrafluoroethylene, a polyvinylchloride (PVC), a polyethersulfone, a polyphenylene oxide, a polyhexamethylene adipamide, a polycarbonate, a polycaprolactam, a polyethylene terephthalate, a polymethylcethacrylate, an acrylonitrile butadiene styrene (ABS), or any combination thereof.

In some embodiments, the matrix is comprised of a flexible material. In some embodiments, the flexible material comprises a dextran, a dextrin, an agarose, a gelatin, a polydimethylsiloxane (PDMS), a silicon, a silicone, a rubber, a collagen, a purified protein, a mixture of different purified proteins, a extrapolysaccharide, a glycosaminoglycan, a cellulose, a chitosan, a hyaluronic acid, an alginate, a polyester, a polyether, a polyanhydride, a polyalkylcyanoacrylate, a polyacrylamide, a polyorthoester, a polyphosphazene, a polyvinylacetate, a block copolymer, a polypropylene, a polytetrafluorethylene (PTFE), a polyurethane, a poly(lactic-co-glycolic acid (PLGA), a copolymer, or any combination thereof.

In some embodiments, the matrix comprises a rigid spherical particle, a polystyrene latex microbead, a magnetic nano- or micro-particle, a nanosized quantum dot, a 4, poly(lactic-co-glycolic acid) (PLGA) microsphere, a non-spherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof.

In some embodiments, the matrix is in the form of a bead. In some embodiments, the bead comprises an average diameter of at least about 1 nanometers (nm) up to 1500 micrometers (μm).

In some embodiments, the matrix further comprises biotin. In some embodiments, the matrix further comprises an ECM composition. In some embodiments, the ECM composition comprises retronectin, fibronectin, laminin, human Fc-delta-like protein 4, solubilized basement membrane preparation extracted from the Engelbreth-Holm-Swarm (EHS) mouse sarcoma, methylcellulose, collagen, elastin, hyaluronic acid, or any combination thereof.

In some embodiments, the matrix is not a cell. In some embodiments, the matrix is not a phospholipid bilayer.

### (6) Pharmaceutical Compositions

Provided herein are pharmaceutical compositions comprising a population of single positive T cells; and a pharmaceutically acceptable excipient. In some embodiments, the population of single positive T cells comprise single positive CD4⁺ T cells. In some embodiments, the population of single positive T cells comprise single positive CD8⁺ T cells. In some embodiments, a pharmaceutical composition provided herein comprises a vehicle suitable for the maintenance of the activated T cells until they are infused into a subject. In embodiments, a pharmaceutical composition comprising a population of T cells provided herein can be administered either alone or together with a pharmaceutically acceptable carrier or excipient, by any routes, and such administration can be carried out in both single and multiple dosages.

In some embodiments, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Additional carriers and excipients can include but are not limited to, for example, dextrose, sodium chloride, sucrose, lactose, cellulose, xylitol, sorbitol, maltitol, gelatin, PEG, PVP, and any combination thereof.

In some embodiments, a pharmaceutical composition provided herein comprises a saline solution. In some embodiments, a pharmaceutical composition provided herein comprises lactated Ringer's Injection Solution, USP. In some embodiments, a pharmaceutical composition provided herein comprises dextrose normal saline (D5NS). In some embodiments, a pharmaceutical composition provided herein comprises human serum albumin (HAS). In some embodiments, an excipient such as dextrose or sodium chloride can be present in a composition at a percent from about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15%.

In some embodiments, a pharmaceutical composition provided herein comprises an additional population of cells. In some embodiments, a pharmaceutical composition provided herein comprises an additional agent. Compositions provided herein can be supplemented to include other immunomodulatory components in addition to the single positive T cells provided herein. Such additional components may be added prior to, concomitant with, or subsequent to the single positive T cells provided herein. The selection of desired time points and dosage concentrations and frequencies at which such supplemental immunomodulatory components are added may be selected according to relevant considerations, such as desired proliferation rate, expansion rate, cell number, longevity, or immunogenicity. Supplemental components can include but are not limited to, for example, one or more leukocytes other than a single positive T cell, cytokines, lymphokines, chemokines, and antibodies. Exemplary leukocytes that may be selected include but are not limited to adherent cells, such as non-CD8 adherent cells, CD14⁺ adherent cells, monocytes, macrophages, helper T cells, memory T cells, natural killer cells, and other leukocytes that may impart an immunomodulatory effect or stimulus. Such leukocytes may be of autologous or heterologous origin. In some embodiments, selected leukocytes are of autologous origin. Exemplary cytokines include interleukins, such as IL-1, IL-2, IL-3, IL-4, IL-5, IL-6, IL-7, IL-8, IL-9, IL-11, IL-12, IL-15, IL-17, IL-21, interferons, such as γ-interferon, and tumor necrosis factors (TNFs), such as TNF-α or CD70, LT, 4-1 BBL and OX40L. The cytokines may be of recombinant or natural origin. In some embodiments, the selected cytokines are of recombinant origin. Exemplary antibodies include monoclonal anti-CD3 antibodies, such as that marked as ORTHOCLONE OKT® 3 (muromonab-CD3).

### (7) Dosing and Administration

In some embodiments, the compositions, single positive CD4⁺ T cells, single positive CD8⁺ T cells, and pharmaceutical compositions provided herein are administered to a subject in need of treatment.

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

The skilled artisan can determine a therapeutically effective amount of cells for administration to a subject in need of treatment. In some embodiments, the single positive CD4⁺ T cells or the single positive CD8⁺ T cells provided herein are administered at a dose of at least about 1×10⁵ cells or more, 1×10⁶ cells or more, at least about 2×10⁶ cells or more, at least about 3×10⁶ cells or more, at least about 4×10⁶ cells or more, at least about 5×10⁶ cells or more, at least about 6×10⁶ cells or more, at least about 6×10⁶ cells or more, at least about 8×10⁶ cells or more, at least about 9×10⁶ cells or more, 1×10⁷ cells or more, at least about 2×10⁷ cells or more, at least about 3×10⁷ cells or more, at least about 4×10⁷ cells or more, at least about 5×10⁷ cells or more, at least about 6×10⁷ cells or more, at least about 6×10⁷ cells or more, at least about 8×10⁷ cells or more, at least about 9×10⁷ cells or more, at least about 1×10⁸ cells or more, at least about 2×10⁸ cells or more, at least about 3×10⁸ cells or more, at least about 4×10⁸ cells or more, at least about 5×10⁸ cells or more, at least about 6×10⁸ cells or more, at least about 6×10⁸ cells or more, at least about 8×10⁸ cells or more, at least about 9×10⁸ cells or more, at least about 1×10⁹ cells or more, at least about 2×10⁹ cells, at least about 3×10⁹ cells, at least about 4×10⁹ cells, at least about 5×10⁹ cells or more, at least about 6×10⁹ cells or more, at least about 6×10⁹ cells or more, at least about 8×10⁹ cells or more, at least about 9×10⁹ cells or more, at least about 1×10¹⁰ cells or more, at least about 2×10¹⁰ cells or more, at least about 3×10¹⁰ cells or more, at least about 4×10¹⁰ cells or more, at least about 5×10¹⁰ cells or more, at least about 6×10¹⁰ cells or more, at least about 6×10¹⁰ cells or more, at least about 8×10¹⁰ cells or more, at least about 9×10¹⁰ cells or more, at least about 1×10¹¹ cells or more, at least about 2×10¹¹ cells or more, at least about 3×10¹¹ cells or more, at least about 4×10¹¹ cells or more, at least about 5×10¹¹ cells or more, at least about 6×10¹¹ cells or more, at least about 6×10¹¹ cells or more, at least about 8×10¹¹ cells or more, at least about 9×10¹¹ cells or more, or at least about 1×10¹² cells to a subject.

In some embodiments, the administering is local or systemic. In some embodiments, the administering is topical administration. In some embodiments, the administering is intravenous. In some embodiments, the administering is intrasplenic. In some embodiments, the administering is via intratumoral injection. In some embodiments, the administering is via intracardiac injection. In some embodiments, the administering is intramuscular. In some embodiments, the administering is subcutaneous. In some embodiments, the administering is every 1, 2, 4, 6, 8, 12, 24, 36, or 48 hours. In some embodiments, the administering is daily, weekly, or monthly. In some embodiments, the administering is repeated at least about every 7 days, 14 days, 21 days, 28 days, 35 days, 42 days, 49 days, 56 days, 63 days, up to 70 days. In some embodiments, a composition or pharmaceutical composition provided herein is administered to the subject by multiple doses.

### (8) Therapeutic Applications

The single positive CD4⁺ and single positive CD8⁺ T cells provided herein are useful in the preparation of immunotherapeutic compositions for the treatment of a disease (e.g., cancer or autoimmune disease).

Provided herein are methods of treating a disease or condition in a subject, the methods comprising: administering to a subject in need thereof a population of single positive CD4⁺ cells provided herein, a population of single positive CD8⁺ cells provided herein, a composition, or a pharmaceutical composition provided herein. In some embodiments, the disease or the condition is a cancer, an autoimmune disease, a blood disorder, an infection, or a metabolic disorder.

In some embodiments, the subject in need thereof has, is at risk of developing, or is diagnosed with a solid tumor or a blood cancer. Examples of cancer include, but are not limited to, carcinoma, lymphoma, blastoma, sarcoma, melanoma, and leukemia. In some embodiments, the cancer is lung cancer, breast cancer, ovarian cancer, colon cancer, pancreatic cancer, prostate cancer, head and neck cancer, skin (melanoma) cancer, bladder cancer, kidney cancer, cervical cancer, uterine cancer, testicular cancer, multiple myeloma, T cell lymphoma, Kaposi's sarcoma, osteosarcoma, neuroblastoma, retinoblastoma, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, lymphoma (Hodgkin's disease, non-Hodgkin's disease), heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, leiomyosarcoma, rhabdomyosarcoma, colon carcinoma, squamous cell carcinoma, basal cell carcinoma, cystadenocarcinoma, medullary carcinoma, bronchogenic carcinoma, renal cell carcinoma, hepatoma, bile duct carcinoma, choriocarcinoma, seminoma, embryonal carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, or meningioma.

In some embodiments, the subject in need thereof has, is at risk of developing, or is diagnosed with a blood disorder or blood condition. In some embodiments, the blood disorder or blood condition is an anemia or a leukopenia. In some embodiments, the blood disorder or blood condition is aplastic anemia, cutaneous T-cell lymphoma (CTCL), severe combined immunodeficiency (SCID), DiGeorge syndrome, Omenn Syndrome, or cartilage-hair hypoplasia.

In some embodiments, the subject in need thereof has, is at risk of developing, or is diagnosed with an infection. In some embodiments, the infection is a viral infection. In some embodiments, the viral infection is a human immunodeficiency virus (HIV) infection.

In some embodiments, the subject in need thereof has, is at risk of developing, or is diagnosed with an autoimmune disease. Such conditions include, but are not limited to, rheumatoid arthritis (RA), systemic lupus erythematosus (SLE), scleroderma, Sjogren's syndrome, multiple sclerosis, inflammatory bowel diseases, for example, ulcerative colitis and Crohn's disease, pulmonary inflammation, asthma, and idiopathic thrombocytopenia purpura (ITP).

In some embodiments, the subject in need thereof has, is at risk of developing, or is diagnosed with a metabolic disorder. In some embodiments, the metabolic disorder is diabetes. In some embodiments, the diabetes is selected from the group consisting of: Type I diabetes, Type II diabetes, Type 1.5 diabetes, pre-diabetes, and gestational diabetes.

In some embodiments, the subject in need thereof is undergoing an organ transplant. In some embodiments, the subject in need thereof has, is at risk of developing, or is diagnosed with graft versus host disease (GVHD).

### (9) Systems and Screening Platforms

Provided herein are systems for use in the production of a population of in vitro-differentiated T cells provided herein. Further provided herein is a system for producing a single positive CD4⁺ T cell comprising: a cell culture vessel configured for localizing a plurality of cell surface receptor binding agents to an in vitro-differentiated double positive (CD4⁺ CD8⁺) T cell or a population thereof comprising: a matrix; a plurality of cell surface receptor binding agents, wherein the plurality of cell surface receptor binding agents are bound to the matrix and comprise: a CD4 receptor binding agent; a TCR binding agent; and an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time, and wherein the cell culture device is configured to facilitate contact between the plurality of cell surface receptor binding agents and the in vitro-differentiated double positive (CD4⁺ CD8⁺) T cell or a population thereof, thereby producing a single positive CD4⁺ T cell within at least about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, up to about 30 days in culture.

Further provided herein is a system for producing a single positive CD8⁺ T cell comprising: a cell culture device configured localizing a plurality of cell surface receptor binding agents to an in vitro-differentiated double positive (CD4⁺ CD8⁺) T cell or a population thereof comprising: a matrix; a plurality of cell surface receptor binding agents, wherein the plurality of cell surface receptor binding agents are bound to the matrix and comprise: a CD8 receptor binding agent; a TCR binding agent; and an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time, and wherein the cell culture device is configured to facilitate contact between the plurality of cell surface receptor binding agents and the in vitro-differentiated double positive (CD4⁺ CD8⁺) T cell or a population thereof, thereby producing a single positive CD8⁺ T cell within at least about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, up to about 30 days in culture.

Provided herein is a bioreactor, wherein the bioreactor comprises a population of single positive T cells provided herein. Further provided herein is a microfluidic device, wherein the microfluidic device comprises a population of single positive T cells provided herein. Further provided herein is a bioreactor, wherein the bioreactor comprises a population of double positive (CD4⁺ CD8⁺) T cells produced by the methods provided herein. Further provided herein is a bioreactor microfluidic device, wherein the microfluidic device comprises a population of double positive (CD4⁺ CD8⁺) T cells produced by the methods provided herein. The systems, bioreactors, and microfluidic devices provided herein can be used to replace animal testing and accelerate drug development by providing a platform for screening drugs for safety and efficacy in a human-like clinically relevant setting. The systems, bioreactors, and microfluidic devices provided herein can be used to scale up the manufacturing of the cell populations provided herein for commercial use in research and development and as cell therapies for the treatment of a disease, disorder, or a condition.

Further provided herein are screening platforms, wherein the screening platforms comprise: a population of single positive CD4⁺ T cells provided herein and a test agent. Further provided herein are screening platforms, wherein the screening platforms comprise: a population of single positive CD8⁺ T cells provided herein and a test agent. Further provided herein are screening platforms, wherein the screening platforms comprise: a population of double positive (CD4⁺ CD8⁺) T cells; and a test agent. Further provided herein are screening platforms, wherein the screening platforms comprise: a mixed population of in vitro-differentiated T cells, wherein the mixed population of in vitro-differentiated T cells comprise: a population of single positive CD4⁺ T cells provided herein and a population of double positive (CD4⁺ CD8⁺) T cells; and a test agent. Further provided herein are screening platforms, wherein the screening platforms comprise: a mixed population of in vitro-differentiated T cells, wherein the mixed population of in vitro-differentiated T cells comprise: a population of single positive CD8⁺ T cells provided herein and a population of double positive (CD4⁺ CD8⁺) T cells; and a test agent.

Assays performed using the screening platform can be conducted to measure effects of a test agent on the population of cells cultured in a cell-culture device (e.g., a bioreactor or as part of an organ-chip system). Assays can be qualitative or quantitative. The screening platform can be used for live cell imaging assays of the population of cells in various conditions. In some embodiments, the test agent is a therapeutic agent for the treatment of a disease or a condition provided herein. In some embodiments, the test agent is an agent that is expected to improve or increase lymphocyte maturation or function. In some embodiments, the screening platform comprises a library of test agents.

### (10) Kits

Provided herein are kits for use in the production of a population in vitro-differentiated T cells provided herein.

In some embodiments, a formulation of a composition described herein is prepared in a single container for administration. In some embodiments, a formulation of a composition described herein is prepared in two containers for administration, separating the population of in vitro-differentiated single positive T cells from the matrix provided herein or the binding agents provided herein.

As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

Provided herein is a kit, wherein the kit comprises: a first container comprising: a population of single positive CD4⁺ T cells provided herein; and a second container comprising: a matrix, a CD4 binding agent, and a TCR binding agent. Further provided herein is a kit, wherein the kit comprises: a first container comprising: a population of double positive (CD4⁺ CD8⁺) T cells provided herein; and a second container comprising: a matrix, a CD4 binding agent, and a TCR binding agent.

Further provided herein is a kit, wherein the kit comprises: a first container comprising: a population of single positive CD8⁺ T cells provided herein; and a second container comprising: a matrix, a CD8 binding agent, and a TCR binding agent. Further provided herein is a kit, wherein the kit comprises: a first container comprising: a population of double positive (CD4⁺ CD8⁺) T cells provided herein; and a second container comprising: a matrix, a CD8 binding agent, and a TCR binding agent.

Further provided herein is a kit, wherein the kit comprises: a first container comprising: a population of double positive (CD4⁺ CD8⁺) T cells provided herein; a second container comprising: a matrix, a CD8 binding agent, and a TCR binding agent; and a third container comprising a matrix, a CD4 binding agent, and a TCR binding agent.

### (11) Exemplary Embodiments

Provided herein are compositions, wherein the compositions comprise: a matrix; a plurality of cell surface receptor binding agents, wherein the plurality of cell surface receptor binding agents are bound to the matrix and comprise: a CD4 receptor binding agent; a T cell receptor (TCR) binding agent; and a population of single positive CD4 T cells (CD4⁺ CD8⁻). Further provided herein are compositions, wherein the CD4 receptor binding agent comprises an antibody or a functional fragment thereof, a small molecule, a protein, a nucleic acid, or an aptamer. Further provided herein are compositions, wherein the TCR binding agent comprises an antibody or a functional fragment thereof, a small molecule, a protein, a nucleic acid, or an aptamer. Further provided herein are compositions, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are compositions, wherein the plurality of cell surface receptor binding agents bind to an single positive CD4+CD8− T cell. Further provided herein are compositions, wherein the CD4 receptor binding agent binds to a CD4 receptor on an in vitro-differentiated double positive (CD4+ CD8+) T cell. Further provided herein are compositions, wherein the CD4 receptor binding agent binds to a CD4 receptor on a single positive CD4+CD8− T cell. Further provided herein are compositions, wherein the CD4 receptor binding agent binds to an amino acid sequence that is at least 80% identical to SEQ ID NO: 1. Further provided herein are compositions, wherein the TCR binding agent binds to a TCR alpha beta chain on an in vitro-differentiated double positive (CD4+ CD8+) T cell. Further provided herein are compositions, wherein the TCR binding agent binds to a TCR alpha beta chain on a single positive CD4+CD8− T cell. Further provided herein are compositions, wherein the TCR binding agent binds to an amino acid sequence that is at least 80% identical to SEQ ID NO: 2 or SEQ ID NOS: 5-7. Further provided herein are compositions, wherein the TCR binding agent binds to an engineered T cell receptor expressed by an engineered T cell. Further provided herein are compositions, wherein the TCR binding agent binds to a gamma delta TCR expressed by an engineered T cell. Further provided herein are compositions, wherein the population of single positive CD4+ T cells are derived from a population of stem cells. Further provided herein are compositions, wherein the stem cells are human induced pluripotent stem cells (iPSCs). Further provided herein are compositions, wherein the stem cells are human embryonic stem cells (ESCs). Further provided herein are compositions, wherein the single positive CD4⁺ T cells are engineered T cells derived by differentiation of engineered iPSCs. Further provided herein are compositions, wherein the population of single positive CD4+ T cells are derived from cells obtained from cord blood, placental cells, bone marrow, blood, or a tissue biopsy. Further provided herein are compositions, wherein the single positive cells CD4+ T cells are obtained by differentiation of genetically modified cells. Further provided herein are compositions, wherein the population of single positive CD4+ T cells are derived from a mesodermal stem cell lineage. Further provided herein are compositions, wherein the population of single positive CD4+ T cells are derived from a CD34+ progenitor cell. Further provided herein are compositions, wherein the stem cells are adult stem cells. Further provided herein are compositions, wherein the adult stem cells are extracted from or isolated from a healthy subject. Further provided herein are compositions, wherein the adult stem cells are extracted from or isolated from a subject that has, is suspected of having, or is at risk of developing a disease. Further provided herein are compositions, wherein the compositions further comprise one or more cytokines. Further provided herein are compositions, wherein the one or more cytokines are selected from the group consisting of: interleukin 7 (IL-7), interleukin 21 (IL-21), interleukin 2 (IL-2), and any combination thereof. Further provided herein are compositions, wherein the population of single positive CD4+ T cells are reversibly bound to the plurality of cell surface receptor binding agents. Further provided herein are compositions, wherein the population of single positive CD4+ T cells express a CD4 receptor and one or more T cell marker. Further provided herein are compositions, wherein the T cell marker is selected from the group consisting of: ZBTB7B (ThPOK), CD3, TBX21, IL-2, IFNG, TNFα, GATA3, IL-4, IL-13, RORC, IL-17, FOXP3, HELIOS, IL-10, BCL6, IL-21, IL-9 and IL-22. Further provided herein are compositions, wherein the matrix is in the form of a bead. Further provided herein are compositions, wherein the bead comprises an average diameter of at least about 1 nanometers (nm) up to 1500 micrometers (μm). Further provided herein are compositions, wherein the matrix further comprises biotin. Further provided herein are compositions, wherein the matrix comprises a flexible material. Further provided herein are compositions, wherein the flexible material comprises a dextran, a dextrin, an agarose, a gelatin, a polydimethylsiloxane (PDMS), a silicon, a silicone, a rubber, a collagen, a purified protein, a mixture of different purified proteins, a extrapolysaccharide, a glycosaminoglycan, a cellulose, a chitosan, a hyaluronic acid, an alginate, a polyester, a polyether, a polyanhydride, a polyalkylcyanoacrylate, a polyacrylamide, a polyorthoester, a polyphosphazene, a polyvinylacetate, a block copolymer, a polypropylene, a polytetrafluorethylene (PTFE), a polyurethane, a poly(lactic-co-glycolic acid (PLGA), a copolymer, or any combination thereof. Further provided herein are compositions, wherein the matrix is in the form of a solid support. Further provided herein are compositions, wherein the solid support comprises a superparamagnetic colloidal material, a metal, an acrylic, a glass, a plastic, a polystyrene, a thermoplastic polymer, a polytetrafluoroethylene, a polyvinylchloride (PVC), a polyethersulfone, a polyphenylene oxide, a polyhexamethylene adipamide, a polycarbonate, a polycaprolactam, a polyethylene terephthalate, a polymethylcethacrylate, an acrylonitrile butadiene styrene (ABS), or any combination thereof. Further provided herein are compositions, wherein the matrix is biocompatible. Further provided herein are compositions, wherein the matrix is biodegradable. Further provided herein are compositions, wherein the composition further comprises an extracellular matrix (ECM) composition. Further provided herein are compositions, wherein the ECM composition comprises fibronectin, vitronectin, retronectin, laminin, collagen, elastin, hyaluronic acid, methylcellulose, or any combination thereof.

Further provided herein are compositions, wherein the compositions comprise: a matrix; a plurality of cell surface receptor binding agents, wherein the plurality of cell surface receptor binding agents are bound to the matrix and comprise: a CD4 receptor binding agent; and a T cell receptor (TCR) binding agent; and a mixed population of in vitro-differentiated T cells, wherein the mixed population of in vitro-differentiated T cells comprise: a population of single positive CD4⁺ T cells; and a population of double positive (CD4⁺ CD8⁺) T cells, wherein the population of single positive CD4⁺ T cells is at least about 10% of the total number of cells within the mixed population of in vitro-differentiated T cells. Further provided herein are compositions, wherein the population of single positive CD4⁺ T cells is at least about 50% of the total number of cells within the mixed population of in vitro-differentiated T cells. Further provided herein are compositions, wherein the population of single positive CD4⁺ T cells is at least about 60% of the total number of cells within the mixed population of in vitro-differentiated T cells. Further provided herein are compositions, wherein the population of single positive CD4+ T cells is at least about 70% of the total number of cells within the mixed population of in vitro-differentiated T cells. Further provided herein are compositions, wherein population of single positive CD4+ T cells is at least about 80% of the total number of cells within the mixed population of in vitro-differentiated T cells. Further provided herein are compositions, wherein the population of single positive CD4+ T cells is at least about 90% of the total number of cells within the mixed population of in vitro-differentiated T cells. Further provided herein are compositions, wherein the CD4 receptor binding agent comprises an antibody or a functional fragment thereof, a small molecule, a protein, a nucleic acid, or an aptamer. Further provided herein are compositions, wherein the TCR binding agent comprises an antibody or a functional fragment thereof, a small molecule, a protein, a nucleic acid, or an aptamer. Further provided herein are compositions, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are compositions, wherein the plurality of cell surface receptor binding agents bind to an single positive CD4⁺ CD8⁻ T cell. Further provided herein are compositions, wherein the CD4 receptor binding agent binds to a CD4 receptor on an in vitro-differentiated double positive (CD4⁺ CD8⁺) T cell. Further provided herein are compositions, wherein the CD4 receptor binding agent binds to a CD4 receptor on an in vitro-differentiated CD4⁺ CD8⁻ T cell. Further provided herein are compositions, wherein the CD4 receptor binding agent binds to an amino acid sequence that is at least 80% identical to SEQ ID NO: 1. Further provided herein are compositions, wherein the TCR binding agent binds to a TCR alpha beta chain on an in vitro-differentiated double positive (CD4⁺ CD8⁺) T cell. Further provided herein are compositions, wherein the TCR binding agent binds to a TCR alpha beta chain on a single positive CD4⁺ CD8⁻ T cell. Further provided herein are compositions, wherein the TCR binding agent binds to an amino acid sequence that is at least 80% identical to SEQ ID NO: 2 or SEQ ID NO: 5-7. Further provided herein are compositions, wherein the TCR binding agent binds to an engineered T cell receptor expressed by an engineered T cell. Further provided herein are compositions, wherein the TCR binding agent binds to a gamma delta TCR expressed by an engineered T cell. Further provided herein are compositions, wherein the population of single positive CD4+ T cells are derived from a population of stem cells. Further provided herein are compositions, wherein the stem cells are human induced pluripotent stem cells (iPSCs). Further provided herein are compositions, wherein the stem cells are human embryonic stem cells (ESCs). Further provided herein are compositions, wherein the single positive CD4⁺ T cells are engineered T cells derived by differentiation of engineered iPSCs. Further provided herein are compositions, wherein the mixed population of in vitro-differentiated T cells are derived from cord blood, placental cells, bone marrow, blood, or a tissue biopsy. Further provided herein are compositions, wherein the mixed population of in vitro-differentiated T cells are derived from genetically modified cells. Further provided herein are compositions, wherein the mixed population of in vitro-differentiated T cells are derived from a mesodermal stem cell lineage. Further provided herein are compositions, wherein the mixed population of in vitro-differentiated T cells are derived from a CD34⁺ progenitor cell. Further provided herein are compositions, wherein the stem cells are human adult stem cells. Further provided herein are compositions, wherein the human adult stem cells are extracted from or isolated from a healthy subject. Further provided herein are compositions, wherein the human adult stem cells are extracted from or isolated from a subject that has, is suspected of having, or is at risk of developing a disease. Further provided herein are compositions, wherein the compositions further comprise one or more cytokines. Further provided herein are compositions, wherein the one or more cytokines are selected from the group consisting of: IL-7, IL-21, IL-2, and any combination thereof. Further provided herein are compositions, wherein the mixed population of in vitro-differentiated T cells are reversibly bound to the plurality of cell surface receptor binding agents. Further provided herein are compositions, wherein the population of single positive CD4⁺ T cells express a CD4 receptor and one or more T cell marker. Further provided herein are compositions, wherein the T cell marker is selected from the group consisting of: ZBTB7B (ThPOK), CD3, TBX21, IL-2, IFNG, GATA3, IL-4, IL-13, RORC, IL-17, FOXP3, HELIOS, IL-10, BCL6, IL-21, IL-9 and IL-22. Further provided herein are compositions, wherein the population of single positive CD4⁺ T cells do not express CD8. Further provided herein are compositions, wherein the matrix is in the form of a bead. Further provided herein are compositions, wherein the bead comprises an average diameter of at least about 1 nanometers (nm) up to 1500 micrometers (μm). Further provided herein are compositions, wherein the matrix further comprises biotin. Further provided herein are compositions, wherein the matrix comprises a flexible material. Further provided herein are compositions, wherein the flexible material comprises a dextran, a dextrin, an agarose, a gelatin, a polydimethylsiloxane (PDMS), a silicon, a silicone, a rubber, a collagen, a purified protein, a mixture of different purified proteins, a extrapolysaccharide, a glycosaminoglycan, a cellulose, a chitosan, a hyaluronic acid, an alginate, a polyester, a polyether, a polyanhydride, a polyalkylcyanoacrylate, a polyacrylamide, a polyorthoester, a polyphosphazene, a polyvinylacetate, a block copolymer, a polypropylene, a polytetrafluorethylene (PTFE), a polyurethane, a poly(lactic-co-glycolic acid (PLGA), a copolymer, or any combination thereof. Further provided herein are compositions, wherein the matrix is in the form of a solid support. Further provided herein are compositions, wherein the solid support comprises a superparamagnetic colloidal material, a metal, an acrylic, a glass, a plastic, a polystyrene, a thermoplastic polymer, a polytetrafluoroethylene, a polyvinylchloride (PVC), a polyethersulfone, a polyphenylene oxide, a polyhexamethylene adipamide, a polycarbonate, a polycaprolactam, a polyethylene terephthalate, a polymethylcethacrylate, an acrylonitrile butadiene styrene (ABS), or any combination thereof. Further provided herein are compositions, wherein the matrix is biocompatible. Further provided herein are compositions, wherein the matrix is biodegradable. Further provided herein are compositions, wherein the composition further comprises an extracellular matrix (ECM) composition. Further provided herein are compositions, wherein the ECM composition comprises fibronectin, vitronectin, retronectin, laminin, collagen, elastin, hyaluronic acid, methylcellulose, or any combination thereof.

Provided herein are compositions, wherein the compositions comprise: a CD4 binding agent tethered to a TCR binding agent. Provided herein are compositions, wherein the compositions comprise: a CD8 binding agent fixed proximal to a TCR binding agent. Further provided herein are compositions, wherein the CD4 binding agent comprises an antibody or a functional fragment thereof, a small molecule, a protein, a nucleic acid, or an aptamer. Further provided herein are compositions, wherein the TCR binding agent comprises an antibody or a functional fragment thereof, a small molecule, a protein, a nucleic acid, or an aptamer. Further provided herein are compositions, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are compositions, wherein the CD4 binding agent binds to an amino acid sequence that is at least 80% identical to SEQ ID NO: 1. Further provided herein are compositions, wherein the TCR binding agent binds to an amino acid sequence that is at least 80% identical to SEQ ID NO: 2 or SEQ ID NOS: 5-7. Further provided herein are compositions, wherein the TCR binding agent binds to a TCR alpha chain, a TCR beta chain, or a TCR alpha and beta chain. Further provided herein are compositions, wherein the TCR binding agent binds to a TCR gamma chain, TCR delta chain, or a TCR gamma and delta chain. Further provided herein are compositions, wherein the CD4 binding agent is at least about 0.1 nanometers proximal to the TCR binding agent.

Provided herein are compositions, wherein the compositions comprise: a CD4 binding agent fixed proximal to a TCR binding agent. Provided herein are compositions, wherein the compositions comprise: a CD8 binding agent tethered to a TCR binding agent. Further provided herein are compositions, wherein the CD8 binding agent comprises an antibody or a functional fragment thereof, a small molecule, a protein, a nucleic acid, or an aptamer. Further provided herein are compositions, wherein the TCR binding agent comprises an antibody or a functional fragment thereof, a small molecule, a protein, a nucleic acid, or an aptamer. Further provided herein are compositions, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are compositions, wherein the CD8 binding agent binds to an amino acid sequence that is at least 80% identical to SEQ ID NO: 3. Further provided herein are compositions, wherein the TCR binding agent binds to an amino acid sequence that is at least 80% identical to SEQ ID NO: 2 or SEQ ID NOS: 5-7. Further provided herein are compositions, wherein the TCR binding agent binds to a TCR alpha chain, a TCR beta chain, or a TCR alpha and beta chain. Further provided herein are compositions, wherein the TCR binding agent binds to a TCR gamma chain, TCR delta chain, or a TCR gamma and delta chain. Further provided herein are compositions, wherein the CD8 binding agent is at least about 0.1 nanometers proximal to the TCR binding agent.

Provided herein are fusion proteins, wherein the fusion proteins comprise: a CD4 binding agent that binds to SEQ ID NO: 1 or a functional fragment thereof; and a TCR binding agent that binds to SEQ ID NO: 2 or a functional fragment thereof.

Provided herein are fusion proteins, wherein the fusion proteins comprise: a CD8 binding agent that binds to SEQ ID NO: 1 or a functional fragment thereof; and a TCR binding agent that binds to SEQ ID NO: 2 or a functional fragment thereof.

Provided herein are in vitro-differentiated T cells, wherein the in vitro-differentiated T cells are bound to a composition provided herein; or a fusion protein provided herein. Further provided herein are in vitro-differentiated T cells, wherein the in vitro-differentiated T cell is a single positive CD4+ T cell, a single positive CD8+ T cell, a double positive CD4+ CD8+ T cell, or a derivative thereof. Further provided herein are in vitro-differentiated T cells, wherein the single positive CD4⁺ T cell expresses a CD4 receptor and one or more marker selected from the group consisting of: ZBTB7B (ThPOK), CD3, TBX21, IL-2, IFNG, GATA3, IL-4, IL-13, RORC, IL-17, FOXP3, HELIOS, IL-10, BCL6, IL-21, IL-9 and IL-22. Further provided herein are in vitro-differentiated T cells, wherein the single positive CD8⁺ T cell expresses a CD8 receptor; and a Notch receptor or a variant thereof, an intracellular Notch (ICN), or an effector molecule of the Notch receptor. Further provided herein are in vitro-differentiated T cells, wherein the effector molecule of the notch receptor comprises ADAM-family metalloprotease (ADAM10), gamma (γ)-secretase, RBP-Jκ, Mastermind-like family (MAML1-3), or a histone acetyltransferase. Further provided herein are in vitro-differentiated T cells, wherein the Notch receptor or the variant thereof comprises a Notch 1 receptor or a functional fragment thereof, a Notch 2 receptor or a functional fragment thereof, or a Notch 3 receptor or a functional fragment thereof.

Provided herein are methods of producing a population of single positive CD4+ T cells from a population of in vitro-differentiated double positive (CD4⁺ CD8⁺) T cells, wherein the methods comprise: contacting a population of in vitro-differentiated (CD4⁺ CD8⁺) T cells for a period of time with a composition comprising: (i) a matrix; (ii) a CD4 receptor binding agent that is bound to the matrix; and (iii) an a TCR binding agent that is bound to the matrix, thereby producing a population of single positive CD4⁺ T cells. Further provided herein are methods, wherein the CD4 receptor binding agent comprises an antibody or a functional fragment thereof, a small molecule, a protein, a nucleic acid, or an aptamer. Further provided herein are methods, wherein the TCR binding agent comprises an antibody or a functional fragment thereof, a small molecule, a protein, a nucleic acid, or an aptamer. Further provided herein are methods, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F (ab′)2, a ds-diabody, a nanobody, a domain antibody, or a bivalent domain antibody. Further provided herein are methods, wherein the methods further comprise: contacting the population of in vitro-differentiated (CD4+ CD8+) T cells with one or more cytokine. Further provided herein are methods, wherein the cytokine is IL-7, IL-21, IL-2, or any combination thereof. Further provided herein are methods, wherein the period of time is at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days. Further provided herein are methods, wherein the methods further comprise an additional step wherein the population of CD4+ single positive T cells are isolated and purified. Further provided herein are methods, wherein the composition further comprises an extracellular matrix (ECM) composition. Further provided herein are methods, wherein the ECM composition comprises fibronectin, vitronectin, retronectin, laminin, collagen, elastin, hyaluronic acid, methylcellulose, or any combination thereof. Further provided herein are methods, wherein the methods further comprise: contacting the single positive CD4⁺ T cells with a second composition comprising: a matrix and further comprising: (i) a CD3 binding agent that is bound to the matrix; (ii) a CD3 binding agent that is bound to the matrix and a CD28 binding agent that is bound to the matrix; or (iii) a CD3 binding agent that is bound to the matrix; a CD28 binding agent that is bound to the matrix; and a CD2 binding agent that is bound to the matrix. Further provided herein are methods, wherein the methods further comprise contacting the single positive CD4+ T cells with a second composition comprising: a matrix and further comprising: (i) a CD3 binding agent that is bound to the matrix; (ii) a CD3 binding agent that is bound to the matrix and a CD28 binding agent that is bound to the matrix; or (iii) a CD3 binding agent that is bound to the matrix; a CD28 binding agent that is bound to the matrix; and a CD2 binding agent that is bound to the matrix. Further provided herein are methods, wherein the methods further comprise: contacting the population of single positive CD4+ T cells with IL-7, IL-2 and IL-21 at a final concentration of at least about 5 ng/mL up to 100 ng/ml. Further provided herein are methods, wherein the matrix is in the form of a bead. Further provided herein are methods, wherein the surface of the matrix is coated with the plurality of antibodies. Further provided herein are methods, wherein the contacting stimulates the population of in vitro-differentiated double positive (CD4⁺ CD8⁺) T cells to increase CD4 expression and decrease CD8 expression relative to a population of double positive (CD4⁺ CD8⁺) T cells that have not been contacted. Further provided herein are methods, wherein the method does not comprise a step of cell sorting. Further provided herein are methods, wherein the population of single positive CD4+ T cells comprises at least 40%, 50%, 60%, 70%, 80%, 90%, up to 100% of the population of total cells relative to the population of in vitro-differentiated double positive T cells. Further provided herein are methods, wherein the in vitro-differentiated double positive (CD4⁺ CD8⁺) T cells are obtained by differentiation of human pluripotent stem cells. Further provided herein are methods, wherein the human pluripotent stem cells are human induced pluripotent stem cells. Further provided herein are methods, wherein the human pluripotent stem cells are human embryonic stem cells. Further provided herein are methods, wherein the in vitro-differentiated double positive (CD4⁺ CD8⁺) T cells are obtained by differentiation of cells obtained from cord blood, placental cells, bone marrow, blood, or a tissue biopsy. Further provided herein are methods, wherein the in vitro-differentiated double positive (CD4⁺ CD8⁺) T cells are obtained by differentiation of genetically modified cells.

Provided herein are methods for producing a single positive CD4⁺ T cell from a double positive (CD4⁺ CD8⁻) T cell, wherein the methods comprise: contacting an in vitro-differentiated double positive (CD4⁺ CD8⁻) T cell with a CD4 binding agent tethered to a TCR binding agent. Further provided herein are methods, wherein the CD4 binding agent comprises an antibody or a functional fragment thereof, a small molecule, a protein, a nucleic acid, or an aptamer. Further provided herein are methods, wherein the TCR binding agent comprises an antibody or a functional fragment thereof, a small molecule, a protein, a nucleic acid, or an aptamer. Further provided herein are methods, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are methods, wherein the CD4 binding agent binds to an amino acid sequence that is at least 80% identical to SEQ ID NO: 1. Further provided herein are methods, wherein the TCR binding agent binds to an amino acid sequence that is at least 80% identical to SEQ ID NO: 2 or SEQ ID NO: 5-7. Further provided herein are methods, wherein the TCR binding agent binds to an engineered T cell receptor expressed by an engineered T cell. Further provided herein are methods, wherein the TCR binding agent binds to a gamma delta TCR expressed by an engineered T cell. Further provided herein are methods, wherein the TCR binding agent binds to a TCR alpha chain, a TCR beta chain, or a TCR alpha and beta chain. Further provided herein are methods, wherein the TCR binding agent binds to a TCR gamma chain, TCR delta chain, or a TCR gamma and delta chain.

Provided herein are a population of single positive CD4⁺ T cells produced by the methods provided herein.

Provided herein are methods for producing a population of single positive CD8⁺ T cells from a population of in vitro-differentiated double positive (CD4⁺ CD8⁺) T cells, the method comprising: contacting a population of in vitro-differentiated CD4⁺ CD8⁺ T cells for a period of time with a composition comprising: (i) a matrix; (ii) a CD8 binding agent that is bound to the matrix; and (iii) a TCR binding agent that is bound to the matrix, thereby producing a population of single positive CD8+ T cells. Further provided herein are methods, wherein the CD8 binding agent comprises an antibody or a functional fragment thereof, a small molecule, a protein, a nucleic acid, or an aptamer. Further provided herein are methods, wherein the TCR binding agent comprises an antibody or a functional fragment thereof, a small molecule, a protein, a nucleic acid, or an aptamer. Further provided herein are methods, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are methods, wherein the methods further comprise: contacting the population of in vitro-differentiated CD4⁻ CD8⁺ T cells with one or more cytokine. Further provided herein are methods, wherein the cytokine is IL-7, IL-21, IL-2, IL-15 or any combination thereof. Further provided herein are methods, wherein the period of time for contacting the population of in vitro-differentiated CD4+ CD8+ T cells is at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days. Further provided herein are methods, wherein the methods further comprise an additional step wherein the population of CD8+ single positive T cells are isolated and purified. Further provided herein are methods, wherein the methods further comprise an additional step wherein after performing the steps provided herein, the cells are contacted with a second composition comprising a matrix and further comprising: a CD3 binding agent that is bound to the matrix; a CD3 binding agent that is bound to the matrix and a CD28 binding agent that is bound to the matrix; or a CD3 binding agent that is bound to the matrix; a CD28 binding agent that is bound to the matrix; and a CD2 binding agent that is bound to the matrix. Further provided herein are methods, wherein the methods further comprise an additional step wherein the population of CD8+ single positive T cells are contacted with a second composition comprising a matrix and further comprising: a CD3 binding agent that is bound to the matrix; a CD3 binding agent that is bound to the matrix and a CD28 binding agent that is bound to the matrix; or a CD3 binding agent that is bound to the matrix; a CD28 binding agent that is bound to the matrix; and a CD2 binding agent that is bound to the matrix. Further provided herein are methods, wherein the composition further comprises an extracellular matrix (ECM) composition. Further provided herein are methods, wherein the ECM composition comprises fibronectin, vitronectin, retronectin, laminin, collagen, elastin, hyaluronic acid, methylcellulose, or any combination thereof. Further provided herein are methods, wherein the method further comprises contacting the single positive CD8+ T cells with one or more cytokines selected from IL-7, IL-21, IL-2 and IL-15, wherein each cytokine is administered at a final concentration of at least about 5 ng/mL up to 100 ng/mL. Further provided herein are methods, wherein the matrix is in the form of a bead. Further provided herein are methods, wherein the surface of the matrix is coated with the plurality of antibodies. Further provided herein are methods, wherein the in vitro-differentiated double positive (CD4+ CD8+) T cells are obtained by differentiation of human pluripotent stem cells. Further provided herein are methods, wherein the human pluripotent stem cells are human induced pluripotent stem cells. Further provided herein are methods, wherein the human pluripotent stem cells are human embryonic stem cells. Further provided herein are methods, wherein the in vitro-differentiated double positive (CD4⁺ CD8⁺) T cells are obtained by differentiation of cells obtained from cord blood, placental cells, bone marrow, blood, or a tissue biopsy. Further provided herein are methods, wherein the in vitro-differentiated double positive (CD4⁺ CD8⁺) T cells are obtained by differentiation of genetically modified cells.

Provided herein are methods for producing a single positive CD8⁺ T cell from a double positive (CD4⁺ CD8⁺) T cell, wherein the methods comprise: contacting an in vitro-differentiated double positive (CD4⁺ CD8⁺) T cell with a CD8 binding agent tethered to a TCR binding agent. Further provided herein are methods, wherein the CD8 binding agent comprises an antibody or a functional fragment thereof, a small molecule, a protein, a nucleic acid, or an aptamer. Further provided herein are methods, wherein the TCR binding agent comprises an antibody or a functional fragment thereof, a small molecule, a protein, a nucleic acid, or an aptamer. Further provided herein are methods, wherein the antibody or the functional fragment thereof comprises: a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are methods, wherein the CD8 binding agent binds to an amino acid sequence that is at least 80% identical to SEQ ID NO: 3. Further provided herein are methods, wherein the TCR binding agent binds to an amino acid sequence that is at least 80% identical to SEQ ID NO: 2 or SEQ ID NOS:−5-7. Further provided herein are methods, wherein the TCR binding agent binds to a TCR alpha chain, a TCR beta chain, or a TCR alpha and beta chain. Further provided herein are methods, wherein the TCR binding agent binds to a TCR gamma chain, TCR delta chain, or a TCR gamma and delta chain.

Provided herein are a population of single positive CD8⁺ T cells produced by the methods provided herein.

Provided herein are methods for the treatment of a disease or a condition in a subject, wherein the methods comprise: administering to a subject in need thereof a composition provided herein or a population of single positive CD4⁺ T cells provided herein, thereby treating the disease of condition. Further provided herein are methods, wherein the disease or the condition is a cancer, an autoimmune disease, or diabetes. Further provided herein are methods, wherein the subject in need thereof has a solid tumor or a blood cancer. Further provided herein are methods, wherein the administering is local or systemic. Further provided herein are methods, wherein the administering is topical administration. Further provided herein are methods, wherein the administering is intravenous. Further provided herein are methods, wherein the administering is via intratumoral injection.

Provided herein are systems for producing a single positive CD4⁺ T cell comprising: a cell culture vessel configured for localizing a plurality of cell surface receptor binding agents to an in vitro-differentiated double positive (CD4⁺ CD8⁺) T cell or a population thereof comprising; a matrix: a plurality of cell surface receptor binding agents, wherein the plurality of cell surface receptor binding agents are bound to the matrix and comprise: a CD4 receptor binding agent; a TCR binding agent; and an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time, and wherein the cell culture vessel is configured to facilitate contact between the plurality of cell surface receptor binding agents and the in vitro-differentiated double positive CD4⁺ CD8⁺ T cell or population thereof, thereby producing a single positive CD4+ T cell within at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days.

Provided herein is a system for producing a single positive CD8+ T cell comprising: a cell culture vessel configured localizing a plurality of cell surface receptor binding agents to an in vitro-differentiated double positive (CD4⁺ CD8⁺) T cell or a population thereof comprising; a matrix; a plurality of cell surface receptor binding agents, wherein the plurality of cell surface receptor binding agents are bound to the matrix and comprise: a CD8 receptor binding agent; a TCR binding agent; and an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time, and wherein the cell culture vessel is configured to facilitate contact between the plurality of cell surface receptor binding agents and the in vitro-differentiated double positive CD4⁺ CD8⁺ T cell or population thereof, thereby producing a single positive CD8⁺ T cell within at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days.

### EXAMPLES

### Example 1: Culture and Expansion of Undifferentiated Human Pluripotent Stem Cells in 2D Adherent and 3D Suspension Culture

Human pluripotent stem cells (hPSCs) were maintained on tissue culture (TC) treated flasks (Corning) coated with Cultrex Stem Cell Qualified Reduced Growth Factor Basement Membrane Extract (Cultrex, R&D Systems) and replenished daily with mTeSR™ Plus Basal Medium supplemented with 1× mTeSR™ Plus Supplement (mTeSR Plus, STEMCELL Technologies) in a 37° Celsius 5% CO₂ incubator. After 3-4 days, a near confluent monolayer of cells were fed with fresh mTeSR Plus about 4-6 hrs prior to being single-cell passaged with warmed Accutase (STEMCELL Technologies). Dissociated hPSCs were resuspended in mTeSR Plus supplemented with 5-20 μM Y-27832 dihydrochloride (TOCRIS®) and plated on TC-treated flasks coated with Cultrex at 1.2-2.2×10⁴ cells/cm². 2D adherent cultures were incubated at 37° C. 5% CO₂ and replenished daily with mTeSR Plus without Y-27832 dihydrochloride until passaging about 3-4 days later. For 3D suspension culture, dissociated hPSCs were then resuspended in mTeSR Plus supplemented with 10 μM Y-27832 dihydrochloride and plated in ultra-low attachment (ULA) 6-well plates (Corning) at 6.0×10⁵ cells/mL in 5 mL of medium. These ULA 6-well plates were then placed on an orbital shaker comprised of an Eppendorf Innova Model 2000 portable platform shaker equipped with an Eppendorf Scientific Shaker Utility Tray rotating inside a 37° C. 5% CO₂ incubator. 3D suspension cultures were replenished daily with StemScale™ PSC Suspension Medium (Gibco) without Y-27832 dihydrochloride and single-cell passaged with warmed Accutase every 3-4 days.

In some experiments, Stage 1 cells were generated using an alternative protocol. To initiate a differentiation, hPSCs were maintained in 2D adherent or 3D suspension cultures and then single-cell dissociated with warmed AccutaseR. Cells were then plated at approximately 1.0×10⁶ cells/mL in 5.5 mL of STEMdiff APEL2 Medium (APEL2, STEMCELL Technologies) supplemented with 5-20 μM Y-27832 dihydrochloride, 10 ng/mL basic fibroblast growth factor (bFGF, Peprotech), 3 μM CHIR 99021 (TOCRIS), 200 nM SB4 (TOCRIS), and 100-250 ng/mL vascular endothelial growth factor (VEGF) in ULA 6-well plates on an orbital shaker rotating at 105 rpm in a 37° C. 5% CO₂ incubator. Cell aggregates were spontaneously formed overnight and medium was replenished without Y-27832 dihydrochloride approximately 24 hours post seeding. For the next two days, medium was replenished daily with APEL2 supplemented with 10 ng/ml bFGF, 200 nM SB4, 2 μM SB 431542 (TOCRIS), and 100-250 ng/mL VEGF. For the next two days, medium was replenished daily with APEL2 supplemented with 10 ng/mL bFGF, 200 nM SB4, and 20-250 ng/mL VEGF. For the next four days, medium was replenished daily with APEL2 supplemented with 10 ng/mL bFGF, 10 ng/mL FMS-like tyrosine kinase 3 ligand (FT3L, Peprotech), 30 ng/mL interleukin-3 (IL-3, Peprotech), 40 ng/mL stem cell factor (SCF, Peprotech), and 20-250 ng/mL VEGF. On days 11 to 13, CD34⁺ floating cells (non-aggregates) were collected from the supernatant. The collected cells were then used immediately as the starting point for the production of Stage II cells or frozen for later use.

### Example 2: Differentiation of hPSC-Derived Stage I Cells (CD34⁺)

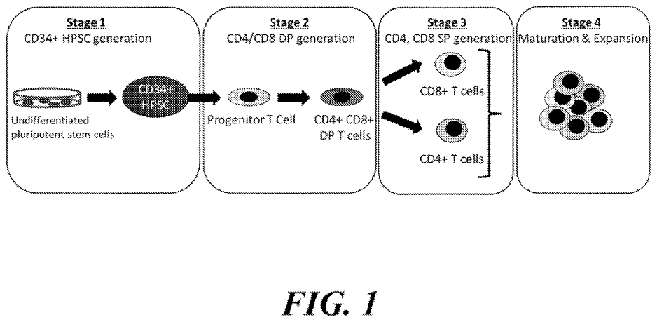

To initiate a differentiation, hPSCs were maintained in 2D adherent or 3D suspension cultures and then single-cell dissociated with warmed AccutaseR. Cells were then plated at approximately 1.0×10⁶ cells/mL in 5.5 mL of STEMdiff APEL2 Medium (APEL2, STEMCELL Technologies) supplemented with 5-20 μM Y-27832 dihydrochloride, 10 ng/mL basic fibroblast growth factor (bFGF, Peprotech), 12.5 ng/ml bone morphogenetic protein 4 (BMP4, Peprotech), 3 μM CHIR 99021 (TOCRIS), 100-400 nM PIK-90 (Selleckchem), and 100-250 ng/mL vascular endothelial growth factor (VEGF) in ULA 6-well plates on an orbital shaker rotating at 105 rpm in a 37° C. 5% CO₂ incubator. Cell aggregates were spontaneously formed overnight and medium is replenished without Y-27832 dihydrochloride approximately 24 hrs post seeding. For the next two days, medium was replenished daily with APEL2 supplemented with 10 ng/ml bFGF, 12.5 ng/ml BMP4, 0.015% dimethyl sulfoxide (DMSO, Sigma-Aldrich), 2 μM SB 431542 (TOCRIS), and 100-250 ng/mL VEGF. For the next two days, medium was replenished daily with APEL2 supplemented with 10 ng/mL bFGF, 12.5 ng/mL BMP4, 0.015% DMSO, and 20-250 ng/ml VEGF. For the next two days, medium was replenished daily with StemPro-34 SFM (Gibco) and 2.4% α-monothioglycerol (α-MTG, Sigma), 50 μg/mL l-ascorbic acid 2-phosphate sesquimagnesium salt hydrate (Ascorbic Acid, Sigma), 1× GlutaMAX™ supplement (Gibco), 1× insulin-transferrin-selenium (ITS-G, Gibco), 10 ng/mL bFGF, 0.015% DMSO, 10 ng/mL FMS-like tyrosine kinase 3 ligand (FT3L, Peprotech), 25 ng/mL insulin-like growth factor 1 (IGF-1, Peprotech), 30 ng/mL interleukin-3 (IL-3, Peprotech), 10 ng/mL interleukin-6 (IL-6, Peprotech), 40 ng/ml stem cell factor (SCF, Peprotech), 30 ng/mL thrombopoietin (TPO, Peprotech), and 20-250 ng/mL VEGF. On day 8, cell aggregates were dissociated with collagenase type II (Gibco) followed by dissociation with TrypLER Express Enzyme (Gibco) and strained through a 100 μm cell strainer (Corning) before magnetic sorting in an EasySep magnet using the EasySep Human CD34 Positive Selection Kit II from StemCELL Technologies according to the manufacturer's protocol. An exemplary differentiation scheme is shown in FIG. 1.

### Example 3: Differentiation of Stage I Cells into Stage II Cells

hPSC-derived CD34⁻ cells were sorted on day 8 of differentiation and plated on non-TC-treated plates (Corning) coated with StemSpan™ Lymphoid Differentiation Coating Material (SCT coating, StemCELL Technologies) at 1.1×10⁴ cells/cm² in complete LYM media (i.e., StemSpan™ SFEM II supplemented with StemSpan™ Lymphoid Progenitor Expansion Supplement (both from StemCELL Technologies)) and incubated in static conditions at 37° C. 5% CO₂. After three days, medium was replenished by topping off the wells with an equal volume of complete LYM media. Three days later, half of the media was removed and then replenished with complete LYM media. Cells were transferred into non-TC-treated plates that are freshly coated with SCT coating. After three days, half of the media was removed and replenished with complete LYM media. At day 21, half of the media was removed and replenished with complete T cell media (i.e., StemSpan™ SFEM II supplemented with StemSpan™ T Cell Progenitor Maturation Supplement (both from StemCELL Technologies) at a cell density of approximately 10.5×10⁴ cells/cm² in freshly SCT-coated non-TC-treated plates. Three days later, half of the media was removed and replenished with complete T cell media. At day 26, half of the media was removed and replenished with complete T cell media at a cell density of approximately 10.5×10⁴ cells/cm² in freshly SCT-coated non-TC-treated plates. For the next two weeks, half media changes were performed every 3 to 4 days until day 40. At day 40, half of the media was removed and replenished with complete T cell media at a cell density of approximately 2.6×10⁵ cells/cm² in freshly SCT-coated non-TC-treated plates. Half media changes were performed every 3-4 days until cells were stimulated for single positive (SP) T cell generation. If cells were not stimulated by day 54, half of the media was removed and replenished with complete T cell media at a cell density of 2.6-5.2×10⁵ cells/cm² in freshly SCT-coated non-TC-treated plates and medium was replenished every 3-4 days until cells were stimulated for SP T cell generation.

In some experiments, Stage II cells were generated using an alternative protocol as follows. 40,000 cells were plated on 12-well non-TC-treated plates (Corning) coated with StemSpan™ Lymphoid Differentiation Coating Material (SCT coating, STEMCELL Technologies). Cells were cultured in medium consisting of IMDM Glutamax Supplement (Thermo Fisher Scientific), 1% BSA, 1×ITS-X (Thermo Fisher Scientific), 0.1% Pluronic F68 (Thermo Fisher Scientific), 20 ng/ml SDF1a (Peprotech), 4.5 mM AA2P (Sigma), 1 or more B-RAF inhibitors, and 1× Stem Cell Tech Lymphoid Supplement (STEMCELL Technologies). The cells were fed every 3-4 days. Six days after seeding cells in the Stage II process, cells were replated at 40,000 cells on 12-well non-TC-treated plates (Corning) coated with StemSpan™ Lymphoid Differentiation Coating Material (SCT coating, STEMCELL Technologies). The cells were cultured in medium consisting of IMDM Glutamax Supplement (Thermo Fisher Scientific), 1% BSA, 1×ITS-X (Thermo Fisher Scientific), 0.1% Pluronic F68 (Thermo Fisher Scientific), 20 ng/ml SDF1a (Peprotech), 4.5 mM AA2P (Sigma), 1 or more B-RAF inhibitor(s), 1 or more Notch signaling inhibitor(s) and 1× Stem Cell Tech T cell supplement (STEMCELL Technologies). The cells were fed routinely every 3-4 days. Cells were replated twice to reduce the density of the culture and Compound E was removed approximately 2 weeks after seeding into T cell supplement containing media. Stage II cells were ready for stimulation approximately 30-40 days after the initiation of the Stage II process. Exemplary components for cell culture medium that can be used are shown in Table 5 below.

TABLE 5
Stage 1-3 Cell Culture Medium Factors.

| Factor Name | Chemical structure (CAS) or Human Amino Acid | |
|---|---|---|
| (acronym) | Sequence NCBI Accession Nos. | Target and/or Mechanism |
| basic fibroblast | NCBI Sequence ID: AAA52534.1; GenBank: | Fibroblast growth factor |
| growth factor (bFGF) | AAB21432.2 | receptor ligand |
| bone morphogenetic | NCBI Reference Sequence: NP_001193.2 | serine-threonine kinase |
| protein 4 (BMP4) | | receptor ligand |
| CHIR99021 | 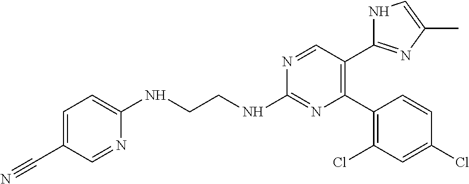 [SMILES: Cc1c[nH]c(-c2cnc(NCCNc3ccc(C#N)cn3)nc2-c2ccc(Cl)cc2Cl)n1] | glycogen synthase kinase 3 (GSK 3) inhibitor |
| | CAS No. 252917-06- | |
| PIK-90 | 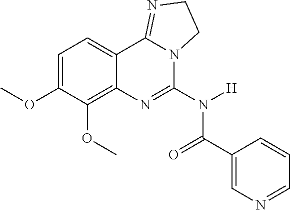 [SMILES: COc1ccc2c(c1OC)N=C(NC(=O)c1cccnc1)N1CCN=C21] | DNA-PK and the PI3K inhibitor |
| | CAS No. 677338-12-4 | |
| vascular endothelial | GenBank: AAA35789.1; Accession: AAA36804.1; | VEGF receptor ligand |
| growth factor (VEGF) | Accession: AAC63102.1; Accession: AAC63101.1 | |
| Y-27632 dihydrochloride | 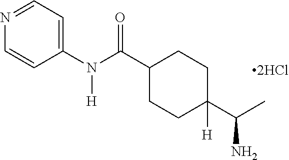 [SMILES: C[C@@H](N)C1CCC(C(=O)Nc2ccncc2)CC1] | Rho kinase inhibitor |
| | CAS No. 129830- | |
| | 38-2 | |
| SB-431542 | 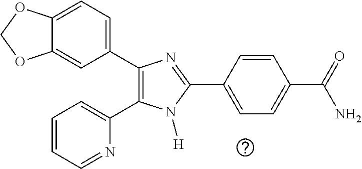 [SMILES: NC(=O)c1ccc(-c2nc(-c3ccc4c(c3)OCO4)c(-c3ccccn3)[nH]2)cc1] | Inhibitor of receptors ALK4, ALK5, and ALK7 |
| | CAS No. | |
| | 301836-41-9 | |
| α-monothioglycerol (α-MTG) | 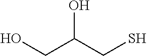 | Stimulates cell proliferation |
| | Cas No. 96-27-5 | |
| L-ascorbic acid (AA2P) | 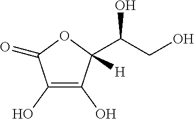 [SMILES: O=C1O[C@H]([C@@H](O)CO)C(O)=C1O] | Antioxidant |
| | Cas No. 50-81-7 | |
| 2-[[(4- Bromophenyl)methyl] thio]benzoxazole (SB4) | 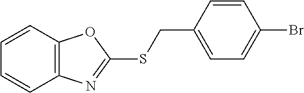 [SMILES: Brc1ccc(CSc2nc3ccccc3o2)cc1] | BMP4 agonist |
| | Cas No. 100874-08-6 | |
| insulin-transferrin- | Insulin: NP_000198.1; NP_001172026.1; | Insulin promotes glucose and |
| selenium (ITS-G) | NP_001172027.1; NP_001278826.1 | amino acid uptake, |
| | Transferrin: NP_001054.2; NP_001341632.2; | lipogenesis, intracellular |
| | NP_001341633.2 | transport, and the synthesis of |
| | Selenium (sodium selenite): | proteins and nucleic acids. |
| | 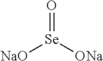 | Transferrin is an iron carrier that reduces oxygen radicals and peroxide. Selenium, as sodium selenite, is a co-factor |
| | CAS No. 10102-18-8 | for glutathione peroxidase |
| | | and other proteins, and is |
| | | used as an anti-oxidant in |
| | | media. |
| FMS-like tyrosine | NP_001191431.1; NP_001191432.1; NP_001265566.1; | A ligand for the FMS-like |
| kinase 3 ligand | NP_001265567.1; NP_001450.2 | tyrosine kinase 3 (FIT3) |
| (FLT3L) | | receptor |
| insulin-like growth | NP_000609.1; NP_001104753.1; NP_001104754.1; | A ligand for the insulin |
| factor 1 (IGF-1) | NP_001104755.1 | growth factor receptor |
| | | (IGFR) |
| interleukin-3 (IL-3) | NP_000579.2 | Ligand for interleukin 3 |
| | | receptor- IL3R |
| interleukin-6 (IL-6) | NP_000591.1; NP_001305024.1; NP_001358025.1 | Ligand for interleukin 6 |
| | | receptor- IL6R |
| Stem Cell Factor | NP_000890.1; 1SCF_A | c-Kit receptor ligand. |
| (SCF) | | |
| thrombopoietin (TPO) | NP_000451.1; NP_001276957.1; NP_001171068.1; | Ligand for MPL proto- |
| | NP_001171069.1 | oncogene, thrombopoietin |
| | | receptor |
| dimethyl sulfoxide (DMSO) | 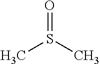 | Diluent, cryoprotectant |
| | CAS No. 67-68-5 | |

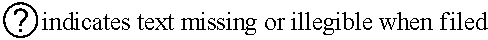

### Example 4: Generation of CD4⁺ SP T Cells from DP Cells/Generation of CD8⁺ SP T Cells from DP Cells

### 4A—Generation of Antibody-Coated Beads.

Beads coated with anti-TCRab and anti-CD4 or anti-CD8 antibodies were generated using anti-biotin MACSiBead™ particles (Miltenyi). For TCRab+CD4 beads, 10 μg of biotinylated anti-human TCRab clone IP26 (Biolegend), and 10 μg of biotinylated anti-human CD4 clone RPA-T4 (Biolegend) were suspended in a volume of 250 μL DPBS (Fisher) and mixed by pipetting. Anti-biotin MACSiBeads™ were pulse-vortexed for 20-30 seconds and then 250 μL of bead suspension was added to the antibody mix. The bead-antibody mix was incubated for 2 hours at 4° C. with constant rotation. Coated beads were stored in the antibody solution at 4° C. for up to 3 months. For TCRab+CD8 beads, the same procedure was followed using biotinylated mouse anti-human CD8a clone SKI (Biolegend).

### 4B—Generation of Coated Plates.

Non-tissue culture treated plastic dishes from Corning® were used in 6-24 well format and T25 format. Plates were coated with retronectin (Takara), retronectin and recombinant human Fc-DLL4 (R&D Systems), or StemSpan™ Lymphoid Differentiation Coating Material (StemCell Technologies). For retronectin coating, plates were coated with DPBS containing 10 μg/mL retronectin at room temperature for 2 hours. For retronectin and hDLL4 coatings, plates were coated with DPBS containing 10 μg/mL retronectin and 10 μg/mL hDLL4 at room temperature for 2 hours. For StemSpan™ coatings, plates were coated with DPBS containing StemSpan™ Lymphoid Differentiation Coating Material at the manufacturer's recommended concentration of 10 μL stock/1 mL PBS. Plates were incubated at room temperature for 2 hours. At the end of the incubation period, plates were washed one time with fresh PBS before use.

### 4C—Stimulation Procedure.

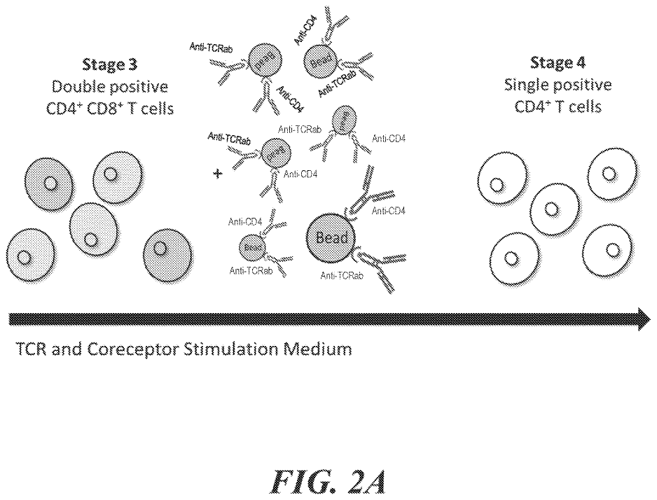

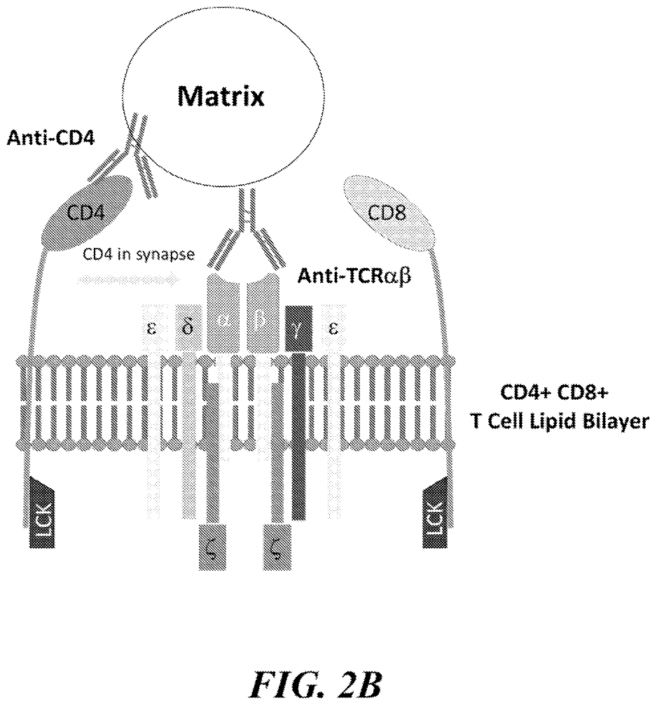

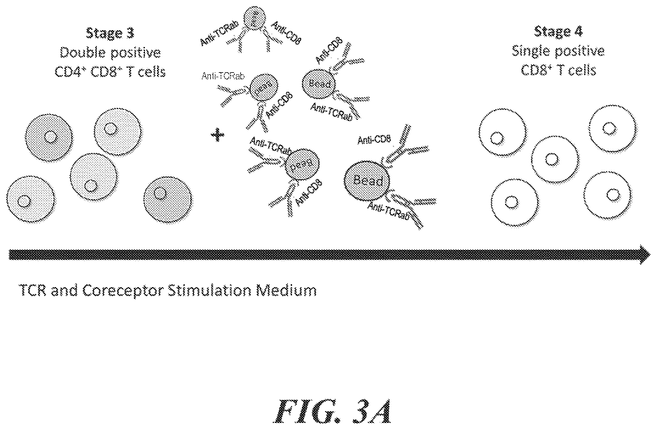

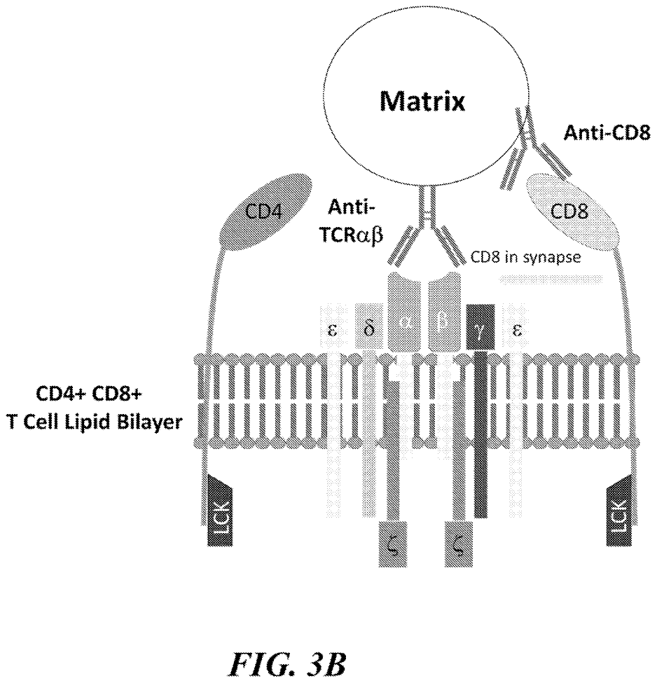

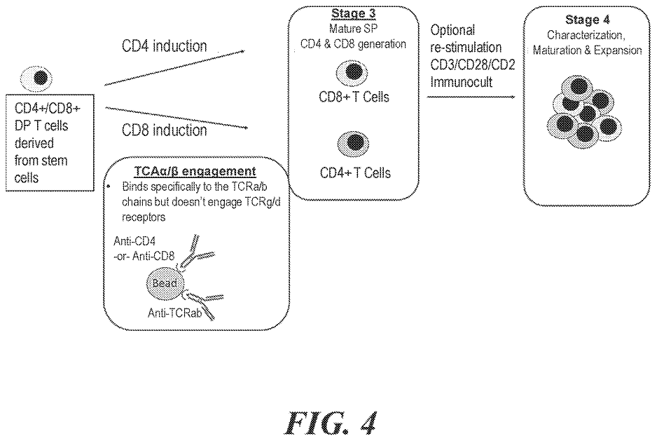

Prior to stimulation, stage II cells expressing CD4 and CD8 were counted and re-suspended in SFEMII⁺ T cell supplement (STEMCELL Technologies) with added IL-7 and IL-21 at 20 ng/mL (cytokines also included IL-2 and IL-15 in some conditions). Beads coated with TCRab⁺ CD4 antibodies or TCRab⁺ CD8 antibodies were added at a ratio of 1 bead: 2 cells and the cells were cultured on retronectin, StemSpan™ Lymphoid Differentiation Coating Material, or retronectin⁺ hDLL4 coated plates for 14 days. The overall procedure for stimulation of (CD4⁺ CD8⁺) double positive T cells to produce single positive CD4⁺ CD8− T cells is shown in FIG. 2A. The engagement of a TCR and CD4 coreceptor is shown in FIG. 2B. The procedure for stimulation of (CD4⁺ CD8⁺) double positive T cells to produce single positive CD8⁺ CD4− T cells is shown in FIG. 3A. The engagement of a TCR and CD8 coreceptor is shown in FIG. 3B. An exemplary stimulation procedure is shown in FIG. 4.

### Example 5: Verification of T Cell Marker Expression by Flow Cytometry

For flow cytometry, 1.0-5.0×10⁵ cells were collected and washed with DPBS. Live/dead stain was done in DPBS with FVS780 (BD Biosciences) or Zombie NIR (Thermo Scientific) stain at 1:1000 or 1:600 respectively for 10 minutes at room temperature in the dark. Cells were then washed with FACS buffer (Invitrogen) and in some conditions FC receptors were blocked by incubation with FACS buffer containing Human TruStain FcX™ (Biolegend) at 5 μL per 100 μL test. Cells were then washed and stained with anti-human CD7-AF700, CD5-PECy7, CD4-BV421, CD8-BV785, CD3-BV510, TCRab-PE, TCRgd-FITC, CD56-PEDazzle, and CD11b-APC at 1:100 in FACS buffer for 20 minutes in the dark at room temperature or 30-60 minutes at 4° C. Cells were then washed with FACS buffer and resuspended in 100-150 μL of FACS buffer and flow data acquired on a Novocyte Quanteon Flow Cytometer (Agilent).

### Example 6: Assessment of Differentiated Single Positive T Cell Marker Expression Profile by Real-Time qPCR

### 6A—RNA Isolation.

RNA was isolated from cell lysates using the Qiagen RNAeasy™ Mini Kit according to the manufacturer's protocol. Briefly, cell aggregates were harvested and lysates obtained by vortexing in RLT buffer from the Qiagen kit. RNA was isolated from cell lysates following multiple washes through a Qiagen isolation column. RNA yield and purity were measured using a NanoDrop One™ (Thermo Scientific).

6B—cDNA First-Strand Synthesis.

cDNA first strand synthesis was performed using the Bio-Rad iScript™ Advanced cDNA Synthesis Kit for RT-qPCR according to the manufacturer's protocol. Briefly, 200 ng of RNA was added to a 1× mixture of iScript™ Advanced Reaction Mix and iScript™ Advanced Reverse Transcriptase and cDNA was generated by a thermocycler (Applied Biosystems) according to the manufacturer's protocol. RNA to cDNA conversion was assumed to be 1:1.

### 6C—SYBR Green RT-qPCR.

SYBR RT-qPCR was performed using 7.5 ng of cDNA mixed with PowerUp SYBR Green™ Master Mix from Applied Biosystems with primers generated by Integrated DNA Technologies according to the manufacturer's protocol. Briefly, 7.5 ng of cDNA was mixed with 1× PowerUp SYBR Green™ Master Mix, 0.4 μM forward and reverse gene primers, and a variable volume of nuclease-free distilled water (Gibco) per sample. These mixtures were added to a MicroAmp Optical 384-well plate (Applied Biosystems), sealed with a MicroAmp Optical Adhesive Film (Applied Biosystems), and RT-qPCR was performed on a QuantStudio 12K Flex using QuantStudio Real-Time PCR Quantification software (Applied Biosystems). CT values were obtained and RQ values were calculated by 2\hat{ }(−delta CT) relative to Stage II (DP T cells).

### 6D—Quantification of ThPOK and RUNX3 Expression.

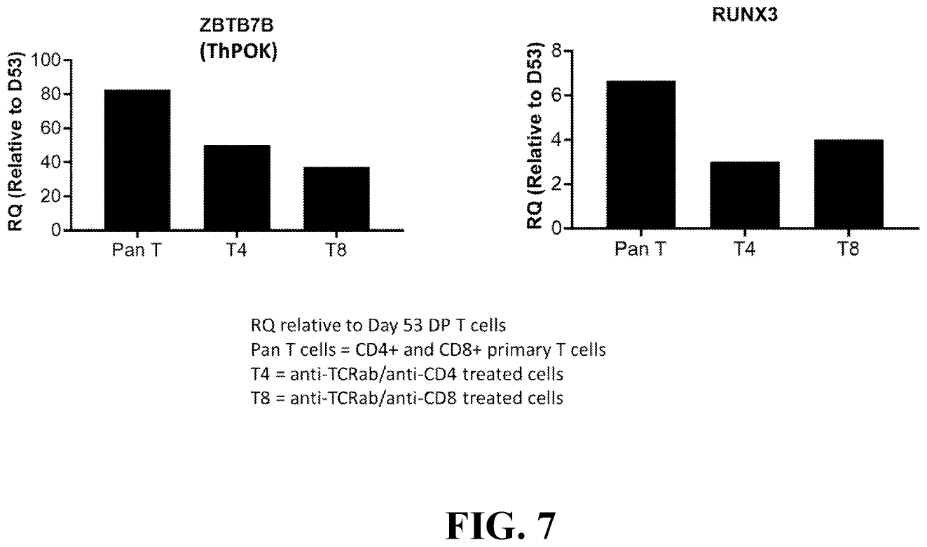

Assessment of expression levels of a CD4⁺ T cell associated transcription factor (ThPOK) and a CD8⁺ T cell associated transcription factor (RUNX3) under CD4⁺ single positive (SP) or CD8⁺ SP inducing conditions was performed using RT-PCR (FIG. 7). Healthy donor T cells comprising both CD4⁺ and CD8⁺ T cells (PanT) were used as a positive control for expression. Under conditions described in Example 4, CD4⁺/CD8⁺ double positive (DP) T cells that were stimulated with anti-CD4/anti-TCRab beads had higher numbers of CD4⁺ T cells at the end of a 14-day culture. When CD4⁺/CD8⁺ DP T cells were stimulated with anti-CD8⁺/anti-TCRab beads, there were less CD4⁺ SPT cells and higher number of CD8⁺ SP T cells. Regardless of the stimulation beads used, some CD4⁺/CD8⁺ DP T cells remained at day 14 of culture. As expected, ThPOK was more highly expressed following the stimulation conditions designed to produce CD4⁺ SP T cells. Conversely, RUNX3 expression was higher following the stimulation conditions designed to produce CD8⁺ SP T cells.

### Example 7: Demonstration of Cytotoxic Activity of the Stage 4 T Cells

### 7A—Cell Lines.

MM.1S/Luciferase cell line (FenicsBio) were cultured in RPMI-1640 medium containing L-glutamine and 10% heat-inactivated FBS. RPMI8226 (ATCC) and RPMI8226/luciferase-mCherry cell lines were grown in RPMI-1640+10% heat-inactivated FBS+/−penicillin and streptomycin.

### 7B—Reagents.

Anti-BCMA-anti-CD3 bispecific antibody and anti-PSMA-anti-CD3 IgG (BPS Bioscience) were diluted to a concentration between 10-300 ng/mL in SFEMII⁺ T cell supplement (STEMCELL Technologies). LDH assay was performed using reagents from the CyQUANT™ LDH Cytotoxicity Assay—Fluorescence Kit (Invitrogen).

### 7C—Cytotoxicity Assay.

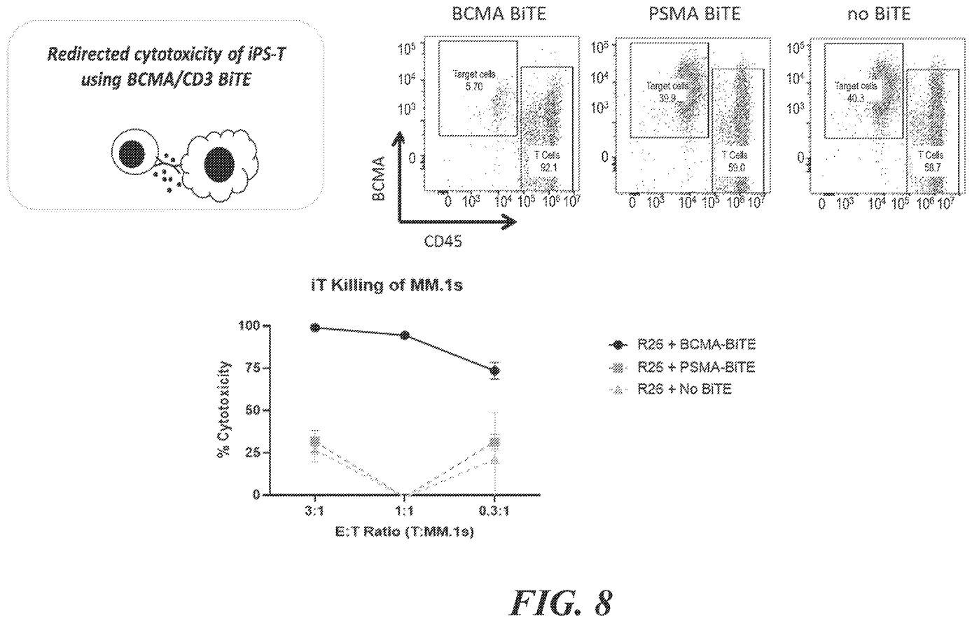

MM.1S were washed once in dPBS and resuspended into SFEMII⁺ T cell supplement (STEMCELL Technologies). 25,000 cells were seeded into a 96-well u-bottom plate. Stimulated T cells were washed once in dPBS and resuspended into SFEMII⁺ T cell supplement (STEMCELL Technologies). The T cells were cocultured with MM.1S at an E:T ratio of 10:1, 5:1, 2.5:1, or 1.25:1 respectively. Control T cells were seeded at a density of 250,000 cells, 125,000 cells, 62,500 cells, or 31,250 cells respectively. Either anti-BCMA-anti-CD3 bispecific antibody (BCMA-BITE) or anti-PSMA-anti-CD3 IgG (PSMA-BiTE) were added to the coculture, and to the control effector only wells. 20 μL of LDH lysis buffer or distilled water (Invitrogen) were added to MM.1S control wells. Cells were incubated at 37° C. for a minimum period of 24 hours to 72 hours. Upon incubation, cells were spun down and supernatant was collected for lactate dehydrogenase assays, while flow cytometry analysis was performed on the remaining cells. Quantification of T cell killing is shown in FIG. 8. MM1.S tumor cells were killed in the presence of anti-BCMA BiTE, while T cells did not clear tumor targets when provided with anti-PSMA BiTE. This data demonstrates successful generation of functional single positive T cells.

### 7D—Lactate Dehydrogenase (LDH) Assay.

The following LDH assay was performed using reagents from the CyQUANT™ LDH Cytotoxicity Assay—Fluorescence Kit (Invitrogen). Briefly, 50 μL of cell supernatant from the coculture, effector only, MM. Is controls were added to a 96-well clear bottom plate (Greiner Bio-One), and mixed in with 50 μL of reagent stock solution. Samples were then incubated at room temperature, protected from the light, for 30 minutes. Post-incubation, 50 μL of stop solution were added to all samples, and the analysis of the plate was performed on a SpectraMax i3× microplate reader (Molecular Devices). Fluorescence was measured at an excitation of 560 nm and an emission of 590 nm.

% cytotoxicity was calculated using the following formula:

$$Cytotoxicity = \frac{\begin{matrix}Fltarget + effector - Fltarget spont . avg - \\ Fleffector spont . avg + FlMedia avg )\end{matrix}}{Fltarget max avg . - Fltarget spont . avg .}$$

### 7E—Flow Cytometry.

For flow cytometry, cells were collected and washed with DPBS. Live/dead stain was done in DPBS with FVS780 (BD Biosciences) stain at 1:1000, and FC receptors were blocked by incubation with DPBS containing Human TruStain FcX™ (Biolegend) at 2.5 μL per 100 μL test for 10 minutes at room temperature in the dark. Cells were then washed with FACS buffer (Invitrogen) and stained with anti-human CD45-PE-Cy7, CD4-APC, CD8-BV785, CD3-BV510, CD269 (BCMA)-PE, and CD69 BV421 at 1:100 in FACS buffer for 30 minutes in the dark at 4° C. Cells were then washed with FACS buffer and resuspended in 100 μl of FACS buffer and flow data acquired on a Novocyte Quanteon™ Flow Cytometer (Agilent).

% survival was calculated as:

$$\% survival = \frac{MM . 1 S in T cell co - culture}{MM . 1 S in media only} * 100$$

7F—Restimulation of SP CD4⁺ T Cells and SP CD8⁺ T Cells.

Single positive cells from Example 4 (after 14 days of stimulation with beads coated with TCRab⁺ CD4 antibodies or TCRab⁺ CD8 antibodies) were stimulated a second time on retronectin coated non-TC treated plates (corning 6-well or T25 format). For these assays, SP T cells were re-suspended in fresh SFEMII⁺ T cell supplement (STEMCELL Technologies) with added IL-7 and IL-21 at 20 ng/ml (cytokines also included IL-2 and IL-15 in some conditions) and re-stimulated with either 12.5 μl/ml ImmunoCult™ Human CD3/CD28/CD2 T Cell Activator (STEMCELL Technologies) or beads coated with TCRab⁺ CD28⁺ CD2 antibodies (1:2 bead: cell ratio). Cultures were replenished by half-feeds twice per week for 14 days. At the end of the assay, phenotype was assessed by flow cytometry as in 7E above (FIG. 5).

### Example 8: Assessment of Factors Expressed by Single Positive iPSC-Derived T Cells

8A—Activation and Expansion of Adult Human Donor T cells.

Isolated human donor T cells were retrieved using CD3 negative selection from liquid nitrogen storage and thawn quickly using a 37° C. water bath (thaw takes approximately 1-2 minutes, and only a small ice chip should remain.) The thawed T cells were added to RPMI media supplemented with 10% AB serum+1× pen/strep (referred to as RH10P) in a 15 ml conical tube in a ratio of approximately 1:10 solution of T cells to RH10P. The conical tube was centrifuged at 300×g for 5 minutes to pellet the cells, supernatant was aspirated and the cells resuspended in 5 mL of RH10P. The cells were then counted, and RH10P was added to the cells to achieve a concentration of approximately 1 million cells/mL. ImmunoCult Activator was added to the media at 25 μl per ml of media. Recombinant human IL-2 was added to the media at 200 units/mL. The plate/flask was left undisturbed in the incubator for three days at 37° C. at 5% CO₂.

On day 3, T cells were gently collected into a conical tube and centrifuged at 300×g for 5 minutes to pellet the cells. The supernatant was aspirated and resuspended in 5 mL of RH10P. Cells were counted. Fresh RH10P was added to the cells to achieve a concentration of approximately 1 million cells/mL, and recombinant human IL-2 was added to the media at 200 units/mL. The plate/flask was left undisturbed in the incubator for 2 days at 37° C. at 5% CO₂.

On day 5, T cells were gently collected into a conical tube and centrifuged at 300×g for 5 minutes to pellet the cells. The supernatant was aspirated and resuspended in 5 mL of RH10P. Cells were counted. Fresh RH10P was added to the cells to achieve a concentration of approximately 1 million cells/mL, and recombinant human IL-2 was added to the media at 200 units/mL. The plate/flask was left undisturbed in the incubator for 2 days at 37° C. at 5% CO₂.

On day 7, T cells were gently collected into a conical tube and centrifuged at 300×g for 5 minutes to pellet the cells. The supernatant was aspirated and resuspended in 5 mL of RH10P. Cells were counted. Fresh RH10P was added to the cells to achieve a concentration of approximately 1 million cells/mL, and recombinant human IL-2 was added to the media at 200 units/mL.

On days 8-10 post-activation, the T cells were evaluated for marker expression. The timing of this step ensured that the cells had time to adequately rest post-activation and expansion.

### 8B—Flow Staining of Adult Donor T Cells and Acquisition of Single Positive In Vitro-Differentiated T Cells.

Compensation was performed using a matrix and corresponding fluorochrome-conjugated antibodies. Adult donor T cells and iPSC-derived double positive (DP) and/or single positive (SP) T cells were collected and the cells were counted. Approximately 100,000 live cells were transferred to a 96-well round bottom plate for cell staining. The cells were centrifuged at 500×g for 5 minutes to form a pellet, and supernatant was removed. The cell pellets were resuspended in 100 μl of cell staining mix corresponding to ligand of interest in Table 6 below.

TABLE 6
Antibody Cocktail Volume per 100,000 cells.

| | Volume per |
|---|---|
| Antibody Cocktail | 100K cells (μl) |
| CD3 BV421 | 1 |
| CD4 BV510 | 1 |
| CD45 BV650 | 1 |
| CD8 BV785 | 1 |
| Ligand of Interest (CD55, | 1 |
| CD59, CD46, CD47, | |
| HLA-E, or HLA-G) PE | |
| FACS Buffer | 95 |

Cells were incubated with the staining mix for 30 minutes at 4° C. 100 μl of FACS Buffer was added to each well and cells centrifuged at 500×g for 5 minutes to pellet the cells. The supernatant was removed from the plate, and the cell pellets were resuspended in 200 μl of fluorescence-activated cell sorting (FACS) buffer. Cells were centrifuged at 500×g for 5 minutes to form a pellet of cells.

The supernatant was removed, and the cell pellets were resuspended in 100 μl of Annexin V Binding Buffer. Next, 2.5 μl of fluorescein isothiocyanate (FITC) Annexin V and 5 μl of 7-AAD viability staining solution were added to each well. The cells were incubated at 4° C. for 15 minutes in the Annexin/staining solution mixture. The cells were then transferred to a NovoCyte Quanteon™ flow cytometer (Agilent Technologies, Inc.). Sample acquisition was set up to collect 90 μl of volume from each well. An acquisition event stop gate was not necessary. After samples were acquired, the acquisition file was saved and exported for analysis.

### 8C—Data Analysis.

Acquisition files from the flow cytometer were imported into a FlowJo worksheet. Cells were gated as follows: Singlets>Lymphocytes>Live cells (Annexin V neg.⁺7-ADD neg.)>T cells (CD3 positive, CD45 positive or CD3⁺ CD45⁺). CD4 and CD8 expression was gated from the T cell gate. Using the T cell gate (CD3 pos.⁺ CD45 pos.), the median fluorescent intensity for the phycoerythrin (PE) channel was calculated. The median fluorescent intensity (MFI) data for the PE channel was imported into GraphPad Prism® (GraphPad Software) for each ligand. The individual ligand MFIs were plotted with a connecting line corresponding to the adult donor T cell expression.

### Example 9: Characterization of Single Positive CD4⁺ T Cells and Single Positive CD8⁺ T Cells

Single positive CD4⁺ cells and single positive CD8⁺ cells were prepared according to Example 4. As a control, double positive (CD4⁺ CD8⁺) T cells were contacted with a bead complexed with isotype antibodies following the methods provided in Example 4. Cells were prepared for flow cytometry according to Examples 7A-7E.

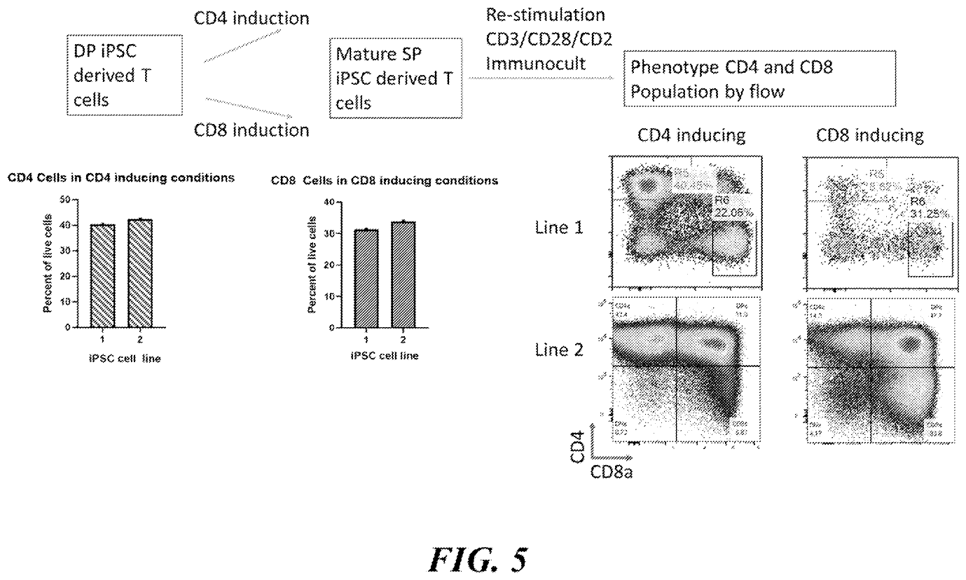

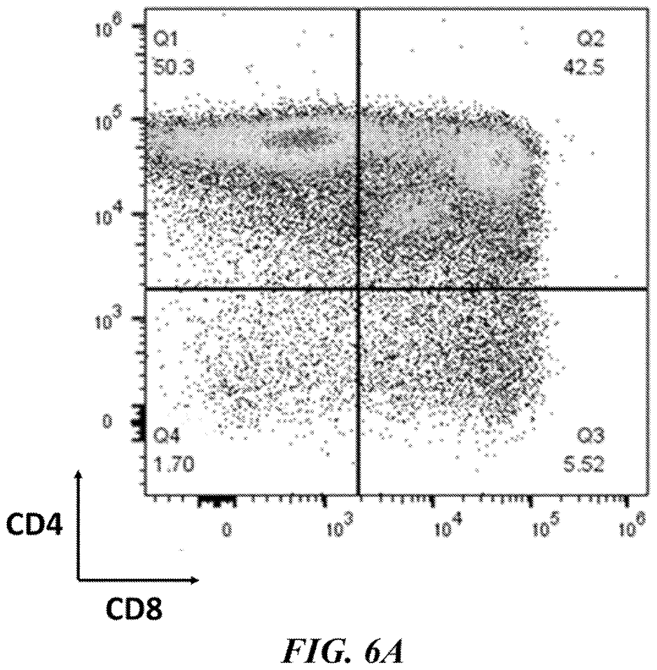

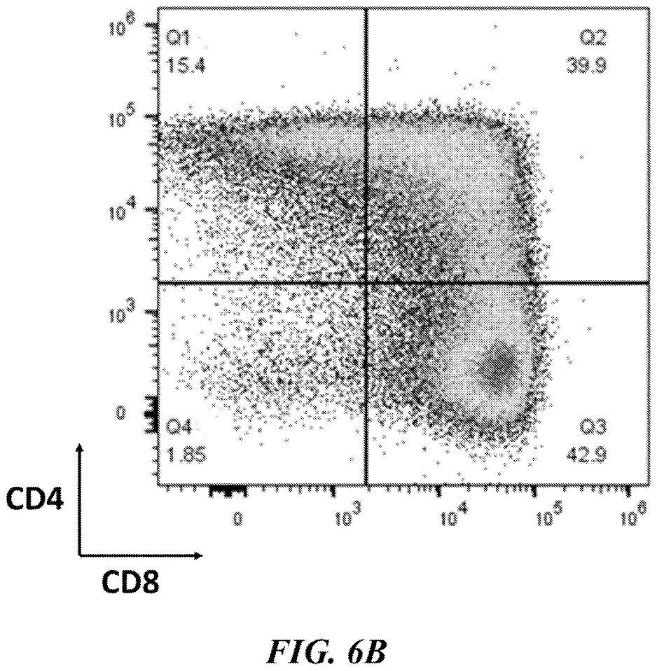

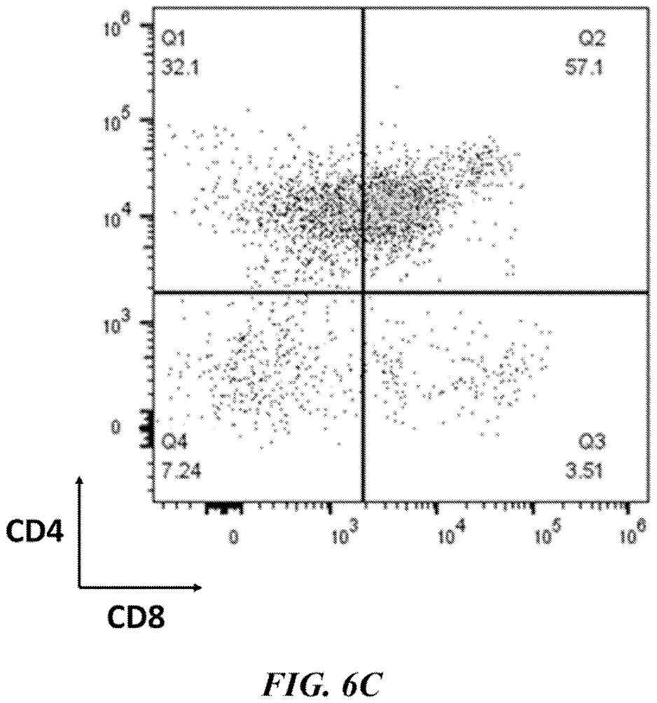

Flow cytometry graphs of the population of T cells that were contacted with a bead complexed with an anti-TCR and anti-CD4 antibody are shown in FIG. 6A. Within the population of T cells analyzed in FIG. 6A, 50.3% were single positive for CD4 and did not express CD8. The population of T cells that were contacted with a bead complexed with an anti-TCR and anti-CD8 antibody are shown in FIG. 6B. Within the population of T cells analyzed in FIG. 6B, 42.9% were single positive for CD8 and did not express CD4. As a negative control, the population of double positive T cells that were contacted with a bead complexed with isotype antibodies is shown in FIG. 6C. The T cells remained double positive for CD4 and CD8.

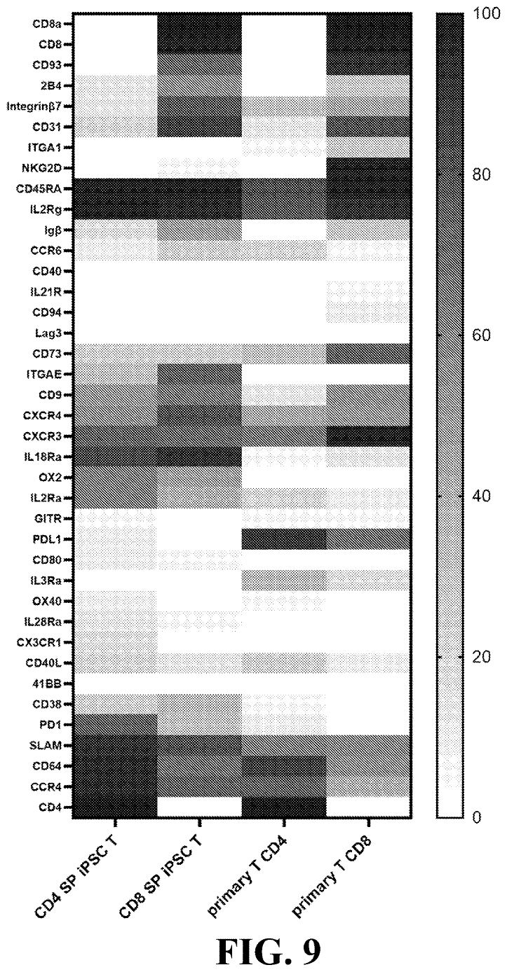

In order to characterize the expression of cell surface markers on CD4⁺ SP T cells generated by the process defined in Example 4, the LEGENDScreen™ Human PE Kit (Biolegend) was used. This kit contains 4-96 well plates that have 364 pre-arranged antibodies, including 10 isotype controls, pre-arrayed in 4 96-well plates. The plates arrive with the antibody array lyophilized and the antibodies are reconstituted in the plate. To examine the expression of the markers on CD4⁺ SP and CD8⁺ iPSC T cells and compare to healthy donor derived CD4⁺ and CD8⁺ T cells, approximately 30 million cells from a CD4⁺ inducing condition and approximately 30 million primary T cells (CD4⁺ and CD8⁺ mixture) were prepared into a single cell solution, labeled with a unique fluorescent barcode, washed and then pooled. The pool of cells was subsequently stained with a live/dead stain and CD4 and CD8 antibodies to discriminate between CD4⁺ and CD8⁺ T cells in the population of cells. After washing, the cells were added to each well of the reconstituted 96-well plates, incubated, washed and then run on a Quanteon flow cytometer (Agilent). Once the samples were collected, the FCS files were analyzed using Novocyte Express Software. As shown in FIG. 9, CD4⁺ SP iPSC-derived T cells have a similar expression pattern of key markers (such as CD4, CD40L, TIM3, and CD64) as do primary CD4+ T cells. Similarly, the CD8⁺ SP T iPSC-derived T cells have a similar expression profile to primary CD8⁺ T cells.

### Example 10: Sorted, CD4+ SP T Cells have Improved Killing in a Repeat Challenge Assay

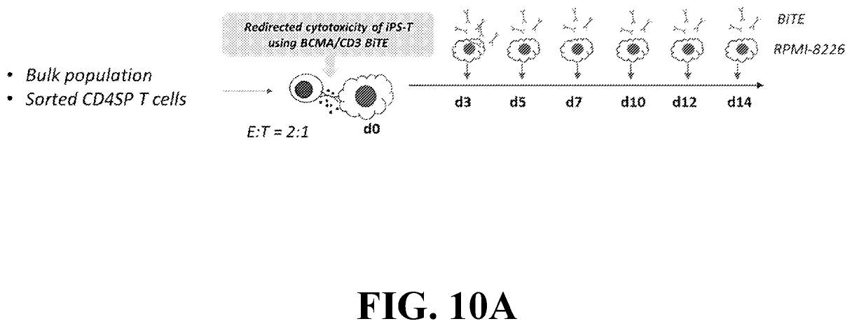

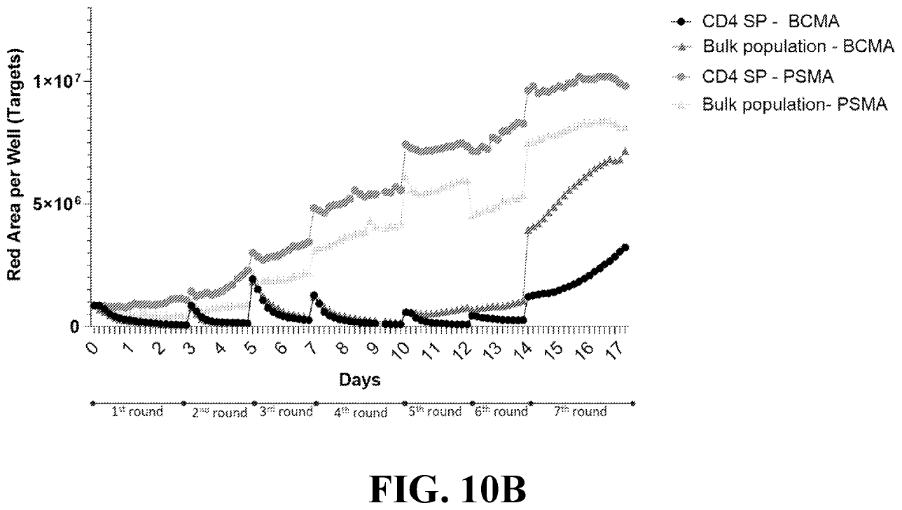

To demonstrate that CD4⁺ SP T cells were functional, either bulk or sorted CD4⁺ T cells were subjected to a tumor killing assay (FIG. 10A). Briefly, iPSC CD4⁺ SP T cell enriched cells were generated as previously described in Example 4 and sorted using the EasySep Human CD4⁺ T cell isolation kit. Using this kit, non-CD4⁺ T cells are targeted for removal using antibodies recognizing specific cell markers. The unwanted cells are labeled with antibodies and magnetic particles and separated without columns using an EasySep magnet. Prior to the cells being purified by the EasySep kit, 1×10⁸ cells had the anti-TCR/anti-CD4 activation beads removed and were cultured overnight. The next day, the cells were counted and enriched using the EasySep kit protocol. Non-sorted or “bulk” cells were set aside prior to the magnetic bead sorting step. After sorting, the enriched cells and “bulk” cells were prepared for functional assessment using a repeat killing tumor challenge assay. Generally, in this assay, effector T cells are incubated with mCherry labeled, BCMA+ tumor targets, RPMI8226 cells at a defined initial E:T ratio. After 2-3 days, the tumor targets are replenished to determine whether the T cells are still capable of killing. Tumor targets are added until the effector T cells lose the ability to kill the additional tumor targets. Tumor growth is monitored by the continual imaging of mCherry fluorescence using the Incucyte platform (Sartorius).

In order to induce tumor killing, a BCMA-CD3 bispecific T cell engager (BiTE) was added to the cells. A non-relevant PSMA-CD3 BiTE was used as a negative control to ensure specificity of killing. A 2:1 effector to target ratio was established on day zero of the killing assay by plating 15,000 target cells in a 96-well plate along with 30,000 effector cells (either CD4⁺ “bulk” iPSC-T cells or sorted CD4⁺ iPSC-T cells). 10 ng/ml of BCMA or PSMA BiTE was added to the cells and the tumor growth was measured using the Incucyte platform. No cytokines were added to the media during the assay. 15,000 tumor targets were added every 2-3 days as a tumor re-challenge. FIG. 10B shows that the effector T cells (both “bulk” and “sorted” CD4⁺ iPSC-T cells) were not able to control the growth of the RPMI-8226 tumor cells in the presence of PSMA BiTE, while the BCMA treated wells were able to control for at least 4 rounds of tumor challenge. In the case of the sorted CD4⁺ iPSC-T cells, tumors were controlled for 6 rounds while the “bulk” cells started to lose full control of the tumor cells after round 4. This data indicates persistent anti-tumor function of the CD4⁺ T cells produced by anti-CD4/anti-TCRab stimulation.

### Example 11: Production and Transcriptional Profiling of CD4⁺ Single Positive T Cells

11a—Cell Stimulation Method.

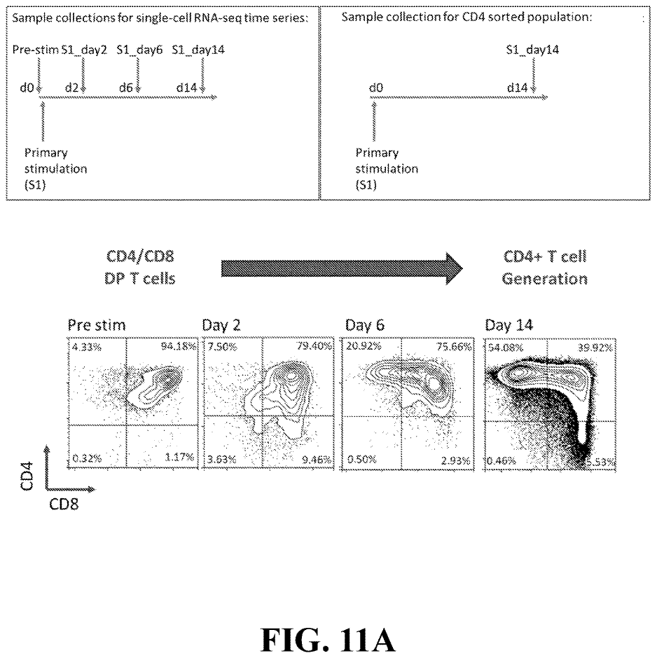

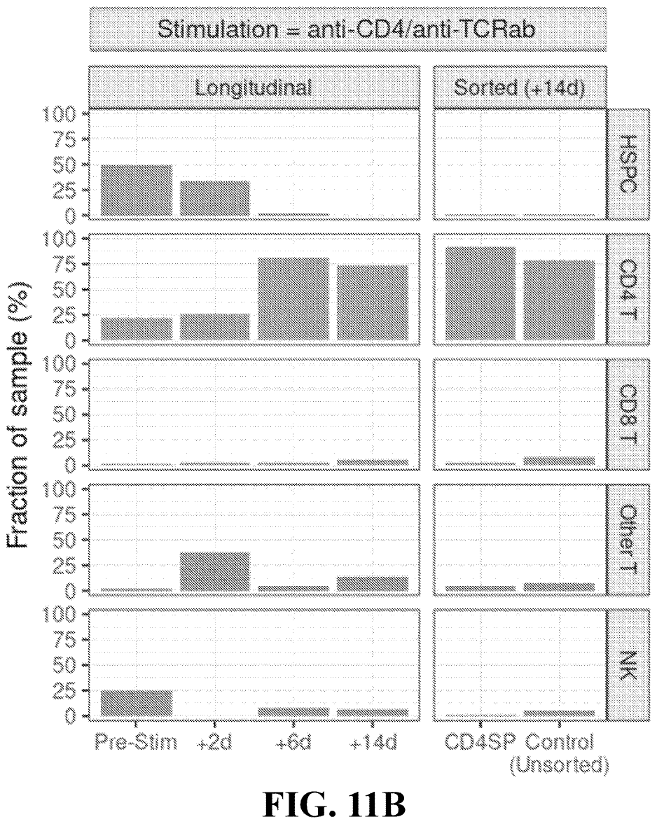

CD4⁺/CD8⁺ double positive cells were stimulated to generate mature iPSC-derived T cells under CD4-inducing conditions. The cells were collected before the stimulation, and at days 2, 6, and 14 during the stimulation. 17 million CD4⁺/CD8⁺ DP cells were plated on a retronectin (Takara) coated (2 ug/cm²) non-TC treated T75 flask (Corning) in stimulation media (IMDM (Gibco), 1% BSA (Proliant), 1×ITSX (Gibco), 0.1% pluronic (Gibco), 4.5 mM AA2P (Sigma), 1× StemSpan™ T Cell Progenitor Maturation Supplement (StemCell Tech 09930), 20 ng/ml CXCL12, 20 ng/ml IL-7, 20 ng/ml IL-21, 10 ng/ml FLT3L (cytokines from Peprotech), and 1 μM BIRB 796 (Tocris)). Stimulation was initiated by adding ProMag HP3 streptavidin beads (Bangs Laboratories) coated with anti-CD4 and anti-TCRab antibodies (Biolegend) at a ratio of 1 bead for every 2 cells. Cultures were fed with fresh media every 3-4 days. Samples were collected for single-cell RNA sequencing prior to stimulation, and on days 2, 6, and 14 of the first stimulation. At each sample collection, cells were de-beaded using a magnet and counted prior to FACS sorting (see below). A repeat of the experiment was performed using an independent differentiation batch with similar results. In the second run, 10 million CD4⁺/CD8⁺ DP cells were plated on a retronectin-coated non-TC treated T75 flask in stimulation media with CD4/TCRab coated beads as above. A sample was collected pre-stimulation and at 14 days post-stimulation. At day 14, CD4⁺ T cells and an un-sorted sample were processed for single-cell RNA sequencing for analysis. FIG. 11A outlines the sample collection time points for each experiment.

### 11B—Cell Sorting.

Live cells were collected for single-cell RNA sequencing (scRNA-seq) at various timepoints during primary stimulation (day 2 (stimulation 1 day 2 (S1D2), d43 of differentiation), day 6 (S1D6, d47), and day 14 (end of primary stimulation (EPR), d55)) by cell sorting using a Sony Cell Sorter (Cell Sorter SH800S, Sony). Briefly, on the day of collection, samples were de-beaded on magnets and counted. 1-2 million cells were collected for cell sorting, stained in 0.5 mL of a 0.83 ug/mL PI solution (Propidium Iodide Solution, BioLegend, 0.5 mg/mL diluted 1:600 in 0.1% BSA in PBS), and strained through a 40 μm filter. PI negative cells were sorted with a 100 μm chip at an average rate of 5000 events per second. Sorted cells were pelleted and resuspended in 0.1% BSA/PBS at a concentration of 1000 cells/uL before proceeding to GEM preparation for scRNA-seq. In a separate experiment, a sample at day 14 of the first stimulation was sorted for CD4⁺ single positive cells. After de-beading and counting, 50 million cells were stained with CD4-FITC and CD8-PE (Biolegend) and cells were sorted with a 100 μm chip at an average rate of 5000 events per second. The resulting three cell populations were pelleted and resuspended as above before proceeding to GEM prep.

### 11C—Next-Generation Sequencing.

Cell counts and viability were quantified using the Countess 3 FL (Thermo Fisher Scientific) with default settings. Subsequently, 5,000-9,000 cells were processed according to the Chromium Next GEM Single Cell 3′ Reagent Kits v3.1 (PN-1000268, 10× Genomics), following the 10× Genomics manual (CG000315 Rev E). The resulting single-cell libraries were assessed on the Bioanalyzer using the High Sensitivity DNA kit (5067-4626, Agilent) and quantified with Qubit™ 1× dsDNA Broad Range (Q33231, Thermo Fisher Scientific). Sequencing libraries were pooled equimolarly for pair-end 150 bp sequencing with 10 bp index reads. The libraries were sequenced on a NovaSeq 6000 S4 flow cell (Illumina Inc) with 20,000 reads per targeted cell.

### 11D—Single-Cell RNA Sequencing Data Processing and Cell Type Annotation.

Sequences were aligned using Cell Ranger version 7.0.0 from 10× Genomics with a GRCh38 assembly (2020-A) used as the reference genome. Cells with fewer than 1000 detected genes and those in which mitochondrial gene expression was at a level above 10% were removed. Cells in the top 5% of cells by number of detected genes as well as those identified as doublets with high confidence by DoubletFinder version 2.0.3 (McGinnis et al., Cell Systems, 2019) were removed. Genes expressed in fewer than 3 cells were also removed.

Seurat version 4.3.0 was used for dataset normalization, integration, dimensionality reduction, clustering, and identification of marker genes, using the sctransform v2 workflow. Datasets were integrated and clustering performed with the following custom (otherwise default) parameters:
SelectIntegrationFeatures (nfeatures=2000)
FindIntegration Anchors (reduction=“rpca”)
IntegrateData (dims=1:30)
FindClusters (resolution=0.6)

Further, a publicly available tool, Azimuth, was applied to assign the cell type annotation to the cells in this sample set (Stuart et al., 2020). Azimuth performs reference-based mapping of the single cell datasets and assigns annotation based on the transcriptomic similarity shared with the reference dataset. Currently, Azimuth offers 14 reference annotation sets to annotate the cells which includes PBMC-10K (Hao and Hao et al., Cell 2021) referred as Human PBMC. Azimuth was applied using RunAzimuth( ) function provided by Seurat package with the default parameters and Human PBMC as a reference set for annotation:
RunAzimuth (reference=“pbmcref”)

The results of this analysis are shown in FIG. 11B and indicate that sorted CD4⁺ SP T cells that were generated by stimulation with anti-TCRab/anti-CD4 beads have a transcriptional profile consistent with CD4⁺ T cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising:
a matrix;
a plurality of cell surface receptor binding agents, wherein the plurality of cell surface receptor binding agents are bound to the matrix and comprise:
a CD4 receptor binding agent;
a T cell receptor (TCR) binding agent; and
a population of single positive CD4 T cells (CD4⁺ CD8⁻).

2. The composition of claim 1, wherein the CD4 receptor binding agent comprises an antibody or a functional fragment thereof, a small molecule, a protein, a nucleic acid, or an aptamer.

3. The composition of claim 1, wherein the TCR binding agent comprises an antibody or a functional fragment thereof, a small molecule, a protein, a nucleic acid, or an aptamer.

4. (canceled)

5. The composition of claim 1, wherein the plurality of cell surface receptor binding agents bind to an single positive CD4⁺ CD8⁻ T cell.

6. The composition of claim 1, wherein the CD4 receptor binding agent binds to a CD4 receptor on an in vitro-differentiated double positive (CD4⁺ CD8⁺) T cell.

7. The composition of claim 1, wherein the CD4 receptor binding agent binds to a CD4 receptor on a single positive CD4⁺ CD8⁻ T cell.

8. The composition of claim 1, wherein the CD4 receptor binding agent binds to an amino acid sequence that is at least 80% identical to SEQ ID NO: 1.

9. The composition of claim 1, wherein the TCR binding agent binds to a TCR alpha beta chain on an in vitro-differentiated double positive (CD4⁺ CD8⁺) T cell or a TCR alpha beta chain on a single positive CD4⁺ CD8⁻ T cell.

10. (canceled)

11. The composition of claim 1, wherein the TCR binding agent binds to an amino acid sequence that is at least 80% identical to SEQ ID NO: 2 or SEQ ID NOS: 5-7.

12. The composition of claim 1, wherein the TCR binding agent binds to an engineered T cell receptor expressed by an engineered T cell or a gamma delta TCR expressed by an engineered T cell.

13. (canceled)

14. The composition of claim 1, wherein the population of single positive CD4⁺ T cells are derived from a population of stem cells.

15. The composition of claim 14, wherein the stem cells are human induced pluripotent stem cells (iPSCs) or human embryonic stem cells (ESC).

16. (canceled)

17. The composition of claim 14, wherein the single positive CD4⁺ T cells are engineered T cells derived by differentiation of engineered iPSCs.

18. The composition of claim 1, wherein the population of single positive CD4⁺ T cells are derived from cells obtained from cord blood, placental cells, bone marrow, blood, or a tissue biopsy.

19. The composition of claim 1, wherein the single positive CD4⁺ T cells are obtained by differentiation of genetically modified cells.

20. The composition of claim 1, wherein the population of single positive CD4⁺ T cells are derived from a mesodermal stem cell lineage or a CD34+ progenitor cell.

21. (canceled)

22. The composition of claim 14, wherein the stem cells are adult stem cells.

23. The composition of claim 22, wherein the adult stem cells are extracted from or isolated from a healthy subject.

24. (canceled)

25. The composition of claim 1, further comprising one or more cytokines.

26. The composition of claim 25, wherein the one or more cytokines are selected from the group consisting of: interleukin 7 (IL-7), interleukin 21 (IL-21), interleukin 2 (IL-2), and any combination thereof.

27. The composition of claim 1, wherein the population of single positive CD4⁺ T cells are reversibly bound to the plurality of cell surface receptor binding agents.

28. The composition of claim 1, wherein the population of single positive CD4⁺ T cells express a CD4 receptor and one or more T cell markers.

29. The composition of claim 28, wherein the one or more T cell markers are selected from the group consisting of: ZBTB7B (ThPOK), CD3, TBX21, IL-2, IFNG, TNFα, GATA3, IL-4, IL-13, RORC, IL-17, FOXP3, HELIOS, IL-10, BCL6, IL-21, IL-9 and IL-22.

30. The composition of claim 1, wherein the matrix is in a form of a bead.

31. The composition of claim 30, wherein the bead comprises an average diameter of at least about 1 nanometers (nm) up to 1500 micrometers (μm).

32. The composition of claim 1, wherein the matrix further comprises biotin.

33. The composition of claim 1, wherein the matrix comprises a flexible material, and wherein the flexible material comprising a dextran, a dextrin, an agarose, a gelatin, a polydimethylsiloxane (PDMS), a silicon, a silicone, a rubber, a collagen, a purified protein, a mixture of different purified proteins, a extrapolysaccharide, a glycosaminoglycan, a cellulose, a chitosan, a hyaluronic acid, an alginate, a polyester, a polyether, a polyanhydride, a polyalkylcyanoacrylate, a polyacrylamide, a polyorthoester, a polyphosphazene, a polyvinylacetate, a block copolymer, a polypropylene, a polytetrafluorethylene (PTFE), a polyurethane, a poly(lactic-co-glycolic acid (PLGA), a copolymer, or any combination thereof.

34. (canceled)

35. The composition of claim 1, wherein the matrix is in a form of a solid support, and wherein the solid support comprises a superparamagnetic colloidal material, a metal, an acrylic, a glass, a plastic, a polystyrene, a thermoplastic polymer, a polytetrafluoroethylene, a polyvinylchloride (PVC), a polyethersulfone, a polyphenylene oxide, a polyhexamethylene adipamide, a polycarbonate, a polycaprolactam, a polyethylene terephthalate, a polymethylcethacrylate, an acrylonitrile butadiene styrene (ABS), or any combination thereof.

36. (canceled)

37. The composition of claim 1, wherein the matrix is biocompatible or biodegradable.

38. (canceled)

39. The composition of claim 1, wherein the composition further comprises an extracellular matrix (ECM) composition, and wherein the ECM composition comprises fibronectin, vitronectin, retronectin, laminin, collagen, elastin, hyaluronic acid, methylcellulose, or any combination thereof.

40. (canceled)

41. A composition comprising:
a matrix;
a plurality of cell surface receptor binding agents, wherein the plurality of cell surface receptor binding agents are bound to the matrix and comprise:
a CD4 receptor binding agent; and
a T cell receptor (TCR) binding agent; and
a mixed population of in vitro-differentiated T cells, wherein the mixed population of in vitro-differentiated T cells comprise:
a population of single positive CD4⁺ T cells; and
a population of double positive (CD4⁺ CD8⁺) T cells,
wherein the population of single positive CD4⁺ T cells is at least about 10% of a total number of cells within the mixed population of in vitro-differentiated T cells.

42.-85. (canceled)

86. A method of producing a population of single positive CD4⁺ T cells from a population of in vitro-differentiated double positive (CD4⁺ CD8⁺) T cells, the method comprising:
contacting a population of in vitro-differentiated (CD4⁺ CD8⁺) T cells for a period of time with a composition comprising:
(i) a matrix;
(ii) a CD4 receptor binding agent that is bound to the matrix; and
(iii) a TCR binding agent that is bound to the matrix,
thereby producing a population of single positive CD4⁺ T cells.

87.-108. (canceled)

109. A population of single positive CD4⁺ T cells produced by the method of claim 86.

110.-113. (canceled)

114. A method for producing a single positive CD4⁺ T cell from a double positive (CD4⁺ CD8⁺) T cell, the method comprising:
contacting an in vitro-differentiated double positive (CD4⁺ CD8⁺) T cell with a CD4 binding agent tethered to a TCR binding agent.

115.-123. (canceled)

124. A method for producing a single positive CD8⁺ T cell from a double positive (CD4⁺ CD8⁺) T cell, the method comprising:
contacting an in vitro-differentiated double positive (CD4⁺ CD8⁺) T cell with a CD8 binding agent tethered to a TCR binding agent.

125.-131. (canceled)

132. A method of producing a population of single positive CD8⁺ T cells from a population of in vitro-differentiated double positive (CD4⁺ CD8⁺) T cells, the method comprising:
contacting a population of in vitro-differentiated CD4⁺ CD8⁺ T cells for a period of time with a composition comprising:
(i) a matrix;
(ii) a CD8 binding agent that is bound to the matrix; and
(iii) a TCR binding agent that is bound to the matrix,
thereby producing a population of single positive CD8⁺ T cells.

133.-151. (canceled)

152. A population of in vitro-differentiated single positive CD8⁺ T cells produced by the method of claim 132.

153.-174. (canceled)

175. A fusion protein comprising a CD4 binding agent that binds to SEQ ID NO: 1 or a functional fragment thereof; and a TCR binding agent that binds to SEQ ID NO: 2 or a functional fragment thereof.

176. A fusion protein comprising a CD8 binding agent that binds to SEQ ID NO: 3 or a T functional fragment thereof; and a TCR binding agent that binds to SEQ ID NO: 2 or a functional fragment thereof.

177.-189. (canceled)

190. A system for producing a single positive CD4⁺ T cell comprising:
a cell culture vessel configured for localizing a plurality of cell surface receptor binding agents to an in vitro-differentiated double positive (CD4⁺ CD8⁺) T cell or a population thereof comprising;
a matrix;
a plurality of cell surface receptor binding agents, wherein the plurality of cell surface receptor binding agents are bound to the matrix and comprise:
a CD4 receptor binding agent;
a TCR binding agent; and
an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time,
and wherein the cell culture vessel is configured to facilitate contact between the plurality of cell surface receptor binding agents and the in vitro-differentiated double positive CD4⁺ CD8⁺ T cell or population thereof, thereby producing a single positive CD4⁺ T cell within at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days.

191. A system for producing a single positive CD8⁺ T cell comprising:
a cell culture vessel configured localizing a plurality of cell surface receptor binding agents to an in vitro-differentiated double positive (CD4⁺ CD8⁺) T cell or a population thereof comprising;
a matrix;
a plurality of cell surface receptor binding agents, wherein the plurality of cell surface receptor binding agents are bound to the matrix and comprise:
a CD8 receptor binding agent;
a TCR binding agent; and
an in vitro-differentiated double positive (CD4⁺ CD8⁺) T cell or a population thereof, wherein the plurality of cell surface receptor binding agents bind to the in vitro-differentiated double positive (CD4⁺ CD8⁺) T cell for a period of time,
and wherein the cell culture vessel is configured to facilitate contact between the plurality of cell surface receptor binding agents and the in vitro-differentiated double positive CD4⁺ CD8⁺ T cell or population thereof, thereby producing a single positive CD8⁺ T cell within at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, up to 20 days.
